(19) United States
(12) Patent Application Publication
(10) Pub. No.: US 2019/0290694 A1
(43) Pub. Date: Sep. 26, 2019

(54) STABLY ENGINEREED PROTEASOME INHIBITOR RESISTANT IMMUNE CELLS FOR IMMUNOTHERAPY

(71) Applicant: CELLECTIS, Paris (FR)

(72) Inventors: Anne-Sophie GAUTRON, Etrechy (FR); Philippe DUCHATEAU, Draveil (FR); Laurent POIROT, Paris (FR); Julien VALTON, New York, NY (US)

(21) Appl. No.: 16/466,327

(22) PCT Filed: Dec. 20, 2017
(86) PCT No.: PCT/EP2017/083932

§ 371 Date: Jun. 4, 2019

Related U.S. Application Data
(60) Provisional application No. 62/437,433, filed on Dec. 21, 2016.

(30) Foreign Application Priority Data
Jan. 20, 2017 (DK) PA201770038

(51) Int. Cl. A61K 35/17; C12N 5/0783; C12N 5/071; A61K 31/69; C07K 14/725

(52) U.S. Cl. CPC A61K 35/17; C12N 5/0636; A61K 45/06; A61K 31/69; C07K 14/7051; C12N 5/0692

(57) ABSTRACT

The present invention relates to gene editing methods to engineer primary immune cells that are made resistant to proteasome inhibitors, such as Bortezomib, Carfilzomib, Ixazomib, Marizomib, Delanzomib or Oporozomib, for their use in cell immunotherapy in combination with proteasome inhibitor treatments.

### FIELD OF THE INVENTION

The present invention relates to methods to engineer primary immune cells that are made resistant to proteasome inhibitors, such as Bortezomib, Carfilzomib, Ixazomib, Marizomib, Delanzomib or Oporozomib, for their use in cell immunotherapy, especially in combination with proteasome inhibitor treatments.

The inventors have developed gene editing techniques for engineering primary immune cell useful in combination therapy. In particular, they set up a method for selectively isolating gene editing events in primary immune cells amounting resistance to proteasome inhibitors by co-transfection into peripheral blood cells of a library of RNA guides with the guided endonuclease Cas9. This method has led to the inactivation of endogenous genes conferring primary immune cells resistance to proteasome inhibitors.

Primary cells have also been made resistant to proteasome inhibitors by expression of exogenous polynucleotide sequences, especially sequences encoding variants of proteasome subunits, such as mutated PSMB proteins.

Among the therapeutic benefits afforded by these resistant immune cells are synergistic effects between chemotherapy and immunotherapy, in a context where the immune cells can also be further modified to allow allogeneic transplantation.

### BACKGROUND OF THE INVENTION

Adoptive cell immunotherapy involves the transfer of immune cells, such as antigen-specific T-cells, generated ex vivo for their infusion into patients. This is one of the promising strategies to treat viral infections and cancer. The cells used for adoptive therapy can be generated either by differentiation of immune cell progenitors, expansion of antigen-specific T-cells or redirection of T-cells through genetic engineering (Park, Rosenberg et al. 2011). For directing T-cells towards specific pathological cells, transgenic T-cell receptors (TCR) or chimeric antigen receptors (CARs) can be successfully expressed at the cell surface, even in the absence of endogenous TCR. These synthetic receptors are consisting of a targeting moiety that is associated with one or more signaling domains in a single fusion molecule or consists of several non-covalently linked transmembrane domains.

In numerous study, the binding moiety of a CAR comprises an antigen-binding domain of a single-chain antibody (scFv), comprising the light and heavy variable fragments of a monoclonal antibody joined by a flexible linker. Binding moieties based on receptor or ligand domains have also been used successfully. Such extracellular domains are linked to signaling domains initially derived from the cytoplasmic region of the CD3zeta, 4-1BB or from the Fc receptor gamma chains.

CARs allow cytotoxic T-cells to be directed against antigens expressed at the surface of tumor cells including lymphomas (Jena, Dotti et al. (2010) Redirecting T-cell specificity by introducing a tumor-specific chimeric antigen receptor. Blood. 116:1035-1044) and destruction of these target cells. The current protocol for the treatment of patients using adoptive immunotherapy is based on autologous cell transfer. Under this approach, T lymphocytes recovered from a given patient, are genetically modified or selected ex vivo, cultivated in vitro in order to amplify the number of cells and finally re-infused into the patient. Autologous therapies face substantial technical and logistic hurdles to practical application, their generation requires expensive dedicated facilities and expert personnel, they must be generated in a short time following a patient's diagnosis, and in many cases, pretreatment of the patient has resulted in degraded immune function, such that the patient's lymphocytes may present in low numbers, may be poorly functional and even dysfunctional. Because of these hurdles, each patient's autologous cell preparation is effectively a new product, resulting in substantial variations in efficacy and safety.

Ideally, one would prefer using cells from healthy individuals engineered to destroy cancer cells. However, T cells from one individual when transferred to another individual can induce a severe immune response, and eventually be rejected. Transferred cells can also recognize the host tissue as foreign, resulting in graft versus host disease (GvHD) and leading to potentially serious tissue damage and death.

The molecular mechanisms responsible for acute or chronic GVHD have been at least partially identified. This is the recognition of MHC disparities between the donor and recipient through specific TCR(s) that can lead to T cells proliferation and to the development of GvHD in recipients of allogeneic cells.

To overcome this problem, new techniques of gene editing have been used to knock out genes encoding the various subunits of the endogenous TCR. So-called “Allogeneic TCR-KO therapeutic cells”, available as “off-the-shelf” therapeutic products, have been produced to be redirected against pathological cells, cancerous, or infected and to induce no or reduced GVHD (Poirot et al. (2015) Multiplex Genome-Edited T-cell Manufacturing Platform for “Off-the-Shelf” Adoptive T-cell Immunotherapies Cancer. Res. 75: 3853-64). Infusion of such TCR-KO cells into patients did not significantly induce GvHD and two pediatric patients suffering refractory AML have been in remission (Leukaemia success heralds wave of gene-editing therapies (2015) Nature 527:146-147).

The survival and/or functioning of these engineered immune primary cells—either autologous or allogeneic—is compromised in the presence of drugs usually used to destroy cancer cells or to deplete immune cells before transplantation. Their concomitant use with chemotherapy is therefore hardly possible. Several attempts to increase the resistance of manufactured T-cells to immune depletion drugs have been described. For instance, the survival and CTL activity of T cells have been proven to resist therapeutic doses of purine analogs by inactivating the activity of dck gene as described in WO201575195.

Meanwhile, other drugs widely used in chemotherapy, such as proteasome inhibitors, can also jeopardize cell immunotherapy treatments. These compounds are known to interact with different components or parts of the proteasome, resulting in cell death, especially after a long term exposure. Bortezomib, is the main protease inhibitor used as a treatment in Multiple Myeloma (MM). This compound binds directly the catalytic site of this enzymatic complex, (Bonvini P., et al. (2007). “Bortezomib-mediated 26S proteasome inhibition causes cell-cycle arrest and induces apoptosis in CD-30+ anaplastic large cell lymphoma” Leukemia 21 (4): 838-42.). Its mechanism of action, although not completely understood, is partly mediated through nuclear factor-kappa B inhibition, resulting in apoptosis, decreased angiogenic cytokine expression, and inhibition of tumor cell adhesion to stroma. Additional mechanisms include c-Jun N-terminal kinase activation and effects on growth factor expression.

Others proteasome inhibitors (PI) such as Carfilzomib, Ixazomib, Marizomib, Delanzomib, Oporozomib were discovered in the last decade, which are now being tested in clinical trials for the treatment of myeloma or of others solid cancers. Meanwhile, these various drugs are also cytotoxic and cannot therefore be easily prescribed in combination with cell therapy.

There is therefore a real need to develop PI resistant immune cells for their use in immunotherapy compatible with proteasome inhibitor treatments.

Here, the inventors have managed to develop primary cells resistant to different proteasome inhibitors, which have the ability of surviving in the presence of therapeutic amounts of proteasome inhibitors, while remaining capable of targeting and killing cancer cells. To meet this achievement, they had to primarily set up a method by which immune cells could be randomly gene edited and screened to identify genomic targets involved into cells sensitivity to proteasome inhibitors. Through such method, they obtained gene edited primary immune cells that exhibit efficient anti-cancer effect and limited side effects. Some engineered cells not only resisted to one PI but to multiple PI and/or to other drugs used for treating relapse refractory cancers, especially myelomas. Furthermore, due to the stability of the genetic modifications induced by the sequence specific endonuclease reagents in the loci selected by the inventors, the cells obtainable by the invention have shown to be genetically stable during their proliferation.

### SUMMARY OF THE INVENTION

The present invention provides methods for engineering primary immune cells to make them resistant to proteasome inhibitors, so that such cells can be used as therapeutic agents in cancer immunotherapy treatments concomitantly with—or subsequently to—proteasome inhibitor treatments.

As part of the present invention is the disclosure of a genome scale gene editing method to identify genes or locus that can confer resistance to immune cells, especially primary immune cells, to toxic compounds, such as proteasome inhibitors. This method more particularly relies on a library of guide RNA or guide DNA, co-transfected in the primary immune cells with a guided endonuclease, such as Cas9 or Cpf1, in a context where the endonuclease induces many different recombination events dictated by the various RNA or DNA guides. The inventors obtained better results when they transduced the immune cells with viral vectors encoding the RNA guides upon artificial CD3 activation of the immune cells on beads. The exact sequence of the RNA or DNA guides could be then rescued from cells that had acquired resistance against the toxic compound by sequencing the viral vectors introduced in these cells. The complementary sequences of the RNA or DNA guides allowed the identification of the genomic loci modified by the RNA-guided endonuclease and the genetic modifications at these loci could be reproduced on other primary cells using same or alternative gene editing methods.

The invention can be practiced on immune cells that can originate from the patients themselves, such as in the case of TIL (Tumor Infiltrating Lymphocytes), in view of operating autologous treatments, or from donors in view of producing allogeneic cells for allogeneic treatments. In the latter case, when the immune cells are more particularly T-cells, the present invention can provide with allogenic gene edited T-cells that are made both resistant to proteasome inhibitors and less alloreactive. In particular, the invention combines gene editing steps leading to inducing cell resistance to proteasome inhibitors, such as into EZH2, PSMB5 and TPPII endogenous genes, with the inactivation of further endogenous genes, such as those encoding T-Cell Receptor (TCR) components, in particular TCRα, TCRβ genes.

As a result, the invention provides primary immune cell that has been stably gene edited to become resistant to a therapeutic effective dose of a proteasome inhibitor (PI).

The present invention encompasses the isolated primary immune cells discoverable and obtainable by the various methods of the invention, in particular gene edited cells that are made resistant to PI by over-expressing HOX, PSMB5 or TPPII genes.

The immune cells of the present invention can further comprise exogenous recombinant polynucleotides, in particular CARs or suicide genes which contribute to improve their specificity towards malignant cells and their efficiency as a therapeutic product, ideally as an “off-the-shelf” product. The present invention also relates to the method for treating or preventing cancer with said gene edited immune cells obtainable by the above methods, especially in combination with proteasome inhibitors.

The invention finally provides therapeutic compositions comprising PI resistant CAR positive immune cells, optionally TCR negative, for their use in patients treated with proteasome inhibitors in anti-cancer therapies.

### BRIEF DESCRIPTION OF THE TABLES AND FIGURES

FIG. 1: Dose response plots to determine EC50 of Bortezomib in immune cells as detailed in Example 1. A: primary T cell. B: primary CAR T-cell (CAR anti-CD123). C: MOLM13 cell line.

FIG. 2: FACS analysis of primary CAR T cells expressing exogenous sequence encoding different proteasome subunit polypeptides POMP, PSMB5 and PSMB5mut as detailed in Example 2.

FIG. 3: FACS analysis of primary CAR T cells expressing exogenous sequence encoding different proteasome subunit polypeptides POMP, PSMB5 and PSMB5mut when cultured with 50 nM Bortezomib as detailed in Example 2.

FIG. 4: Viability plots of the CAR T cells expressing exogenous sequence encoding different proteasome subunit polypeptides POMP, PSMB5 and PSMB5mut as detailed in Example 3. Increased EC50 of Bortezomib is observed in the primary CART gene edited cells according to the invention.

FIG. 5: Efficiency of transduction of genome scale lentiviral library encoding RNA guides into primary T-cells as detailed in Example 4. A—Puromycine sensitivity of Mock- and GECKO-transduced T-cells using the conventional activation/transduction protocol. B—Puromycine sensitivity of Mock- and GECKO-transduced T-cells using the simultaneous activation/transduction step as per the present invention.

Table 1: ISU domain variants from diverse viruses.

Table 2: Amino acid sequences of FP polypeptide from natural and artificial origins.

Table 3: List of genes involved into immune cells inhibitory pathways, which can be advantageously modified or inactivated by inserting exogenous coding sequence in the proteasome inhibitors resistant cells according to the invention.

Table 4: Treatment(s) combined to CAR-expressing engineered immune cells and a proteasome inhibitor

Table 5: sequences referred to in the examples.

Table 6: List of human genes that are up-regulated upon T-cell activation (CAR activation sensitive promoters), in which gene targeted insertion is sought according to the present invention to improve immune cells therapeutic potential.

Table 7: Selection of genes that are steadily transcribed during immune cell activation (dependent or independent from T-cell activation).

Table 8: Selection of genes that are transiently upregulated upon T-cell activation.

Table 9: Selection of genes that are upregulated over more than 24 hours upon T-cell activation.

### DETAILED DESCRIPTION OF THE INVENTION

Unless specifically defined herein, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention is drawn to a general method of preparing primary immune cells for cell immunotherapy involving gene inactivation or genetic targeted integration of an exogenous coding sequence into the chromosomal DNA of said immune cells in order to make them resistant to drugs, especially proteasome inhibitors (PI). According to some aspects, genetic integration is performed in such a way that a polynucleotide sequence encoding a polypeptide, which expression confers resistance to proteasome, is placed under transcriptional control of at least one promoter endogenous to said cells.

Also as a primary object of the present invention is an immune cell that has been stably gene edited to become resistant to a therapeutic effective dose of a proteasome inhibitor (PI).

The term “proteasome inhibitor” as used herein refers to any substance which specifically inhibits proteasome function. Preferred proteasome inhibitors are approved therapeutic products—or under clinical trials—known to interact with proteasome, in particular peptide boronate, such as bortezomib (MLN 9708, CEP18770), epoxyketone derivatives, such as Carfilzomib (ONX 0912), salinosporamide A derivatives, such as Marizomib (NPI-0052) are preferred, especially one selected from the list consisting of bortezomib, carfilzomib, ixazomib, marizomib, delanzomib, oporozomib. Therapeutic forms referred to as bortezomib sc carfilzomib iv or ixazomib po. are even more preferred. Non-polypeptide proteasome inhibitors are also preferred PI according to the invention, such as the natural products lactacystin and Epoxomicin, as well as synthetic coumpounds, such as Disulfiram, Epigallocatechin-3-gallate MG132 (CAS ref.: 133407-82-6) and Beta-hydroxy beta-methylbutyrate.

As used herein, a cell is made “resistant” or “becomes resistant” when it can proliferate and/or survive in standard culture conditions to a dose of a chemically defined compound that usually kills a majority of such cells, preferably an unmodified sister cell or of the same type of cell. In the case of primary immune cells, since some variability may occur between individuals, comparison can be made with other hematopoietic derived immune cells. Generally, the majority of the non gene-edited cells is killed when the compound is provided at a dose equal or superior to that referred to as LD50 (Lethal Dose 50). Preferably, the cell is made resistant to a dose that usually kills 95%, more preferably 99% of a wild type cell population, also referred to as LD95 or LD99. More preferably the cell is made resistant to a “effective therapeutic dose” of said product. This means that the cells can proliferate into a culture medium at a concentration corresponding to that found in the serum of a patient treated with a minimal dose of the product prescribed for a given indication as provided by European public assessment reports (EPAR) for human medicines published by the European Medicines Agency (http://www.ema.europa.eu), even more preferably to the median effective dose (EC50) of said compound, so that the engineered cell can resist to said treating dose in-vivo. More generally, the median LD50 or EC50 that can be measured for the resistant gene edited cells for said compound is on average significantly increased in comparison with that of non gene-edited cells, usually by more than 10%, more usually by more than 20% or even by more than 50%

The term “stable” is applied to the genetic mutation(s), insertion(s) or deletion(s) which can be transmitted from mother to daughter cells without further significant modification at the targeted locus. In general, such unexpected changes remain below one in 10⁵ cells, more generally below one in 10⁶ cells, mostly below one in 10⁷ cells. Off-site modifications through the cells genome are also rare and below the frequencies indicated above.

### Improving the Therapeutic Potential of Immune Cells by Gene Inactivation or Targeted Integration

### General Approach

Gene editing techniques using polynucleotide sequence-specific reagents, such as rare-cutting endonucleases, have become the state of the art for the introduction of genetic modifications into primary cells. However, they have not been used so far in immune cells to introduce exogenous coding sequences that confer resistance to proteasome inhibitors.

The present invention aims to improve the therapeutic potential of immune cells through gene editing techniques, especially by gene targeted integration.

By “gene targeting integration” is meant any known site-specific methods allowing to insert, replace or correct a genomic sequence into a living cell. According to a preferred aspect of the present invention, said gene targeted integration involves homologous gene recombination at the locus of the targeted gene to result the insertion or replacement of at least one exogenous nucleotide, preferably a sequence of several nucleotides (i.e. polynucleotide), and more preferably a coding sequence.

By “sequence-specific reagent” is meant any active molecule that has the ability to specifically recognize a selected polynucleotide sequence at a genomic locus, preferably of at least 9 bp, more preferably of at least 10 bp and even more preferably of at least 12 pb in length, in view of modifying said genomic locus. According to a preferred aspect of the invention, said sequence-specific reagent is preferably a sequence-specific nuclease reagent.

A “Cell” according to the present invention refers, for example, to a cell of hematopoietic origin functionally involved in the initiation and/or execution of innate and/or adaptive immune response. In one preferred embodiment, cells according to the present invention are PBMCs or populations of immune cells derived from PBMCs obtained from a donor. In one preferred embodiment, a cell according to the present invention is a T-cell, preferably obtained from a donor. Said T-cell according to the present invention can be derived from a stem cell. A stem cell can be an adult stem cell, an embryonic stem cell, more particularly a human stem cell, a cord blood stem cell, a progenitor cell, a bone marrow stem cell, a totipotent stem cell or a hematopoietic stem cell. In one embodiment, a human stem cell is a CD34+ cell. Said isolated cell can also be a dendritic cell, killer dendritic cell, a mast cell, a NK-cell, a B-cell or a T-cell selected from the group consisting of inflammatory T-lymphocytes, cytotoxic T-lymphocytes, or helper T-lymphocytes. In a preferred embodiment, said cell can be derived from the group consisting of CD4+T-lymphocytes and CD8+T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T-cell lines available and known to those skilled in the art, may be used. In another embodiment, said cell is preferably derived from a healthy donor. In another embodiment, said cell is part of a mixed population of cells which present different phenotypic characteristics.

By “immune cell” is more particularly meant a cell of hematopoietic origin functionally involved in the initiation and/or execution of innate and/or adaptative immune response, such as typically CD3 or CD4 positive cells as referred to above. For the purpose of the present invention, especially for treating cancer and infection, such an immune cell is preferably not a regulatory T-cell.

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells. Primary cells are generally used in cell therapy as they are deemed more functional and less tumorigenic.

In general, primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284).

The primary immune cells according to the present invention can also be differentiated from stem cells, such as cord blood stem cells, progenitor cells, bone marrow stem cells, hematopoietic stem cells (HSC) and induced pluripotent stem cells (iPS).

By “nuclease reagent” is meant a nucleic acid molecule that contributes to an nuclease catalytic reaction in the target cell, preferably an endonuclease reaction, by itself or as a subunit of a complex such as a guide RNA/Cas9, preferably leading to the cleavage of a nucleic acid sequence target.

The nuclease reagents of the invention are generally “sequence-specific reagents”, meaning that they can induce DNA cleavage in the cells at predetermined loci, referred to by extension as “targeted gene”. The nucleic acid sequence which is recognized by the sequence specific reagents is referred to as “target sequence”. Said target sequence is usually selected to be rare or unique in the cell's genome, and more extensively in the human genome, as can be determined using software and data available from human genome databases, such as http://www.ensembl.org/index.html.

“Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

According to a preferred aspect of the invention, said endonuclease reagent is a nucleic acid encoding an “engineered” or “programmable” rare-cutting endonuclease, such as a homing endonuclease as described for instance by Arnould S., et al. (WO2004067736), a zing finger nuclease (ZFN) as described, for instance, by Urnov F., et al. (Highly efficient endogenous human gene correction using designed zinc-finger nucleases (2005) Nature 435:646-651), a TALE-Nuclease as described, for instance, by Mussolino et al. (A novel TALE nuclease scaffold enables high genome editing activity in combination with low toxicity (2011) Nucl. Acids Res. 39(21):9283-9293), or a MegaTAL nuclease as described, for instance by Boissel et al. (MegaTALs: a rare-cleaving nuclease architecture for therapeutic genome engineering (2013) Nucleic Acids Research 42 (4):2591-2601).

According to another embodiment, the sequence specific reagent is a RNA-guide to be used in conjunction with a RNA guided endonuclease, such as Cas9 or Cpf1, as per, inter alia, the teaching by Doudna, J., and Chapentier, E., (The new frontier of genome engineering with CRISPR-Cas9 (2014) Science 346 (6213):1077), which is incorporated herein by reference.

According to a preferred aspect of the invention, the endonuclease reagent is transiently expressed into the cells, meaning that said reagent is not supposed to integrate into the genome or persist over a long period of time, such as be the case of RNA, more particularly mRNA, proteins or complexes mixing proteins and nucleic acids (eg: Ribonucleoproteins).

In general, 80% the endonuclease reagent is degraded by 30 hours, preferably by 24, more preferably by 20 hours after transfection.

An endonuclease under mRNA form is preferably synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131(18):6364-5).

In general, electroporation steps that are used to transfect immune cells are typically performed in closed chambers comprising parallel plate electrodes producing a pulse electric field between said parallel plate electrodes greater than 100 volts/cm and less than 5,000 volts/cm, substantially uniform throughout the treatment volume such as described in WO2004083379, which is incorporated by reference, especially from page 23, line 25 to page 29, line 11. One such electroporation chamber preferably has a geometric factor (cm⁻¹) defined by the quotient of the electrode gap squared (cm2) divided by the chamber volume (cm³), wherein the geometric factor is less than or equal to 0.1 cm⁻¹, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

Due to their higher specificity, TALE-nuclease have proven to be particularly appropriate sequence specific nuclease reagents for therapeutic applications, especially under heterodimeric forms—i.e. working by pairs with a “right” monomer (also referred to as “5′” or “forward”) and ‘left” monomer (also referred to as “3″” or “reverse”) as reported for instance by Mussolino et al. (TALEN® facilitate targeted genome editing in human cells with high specificity and low cytotoxicity (2014) Nucl. Acids Res. 42(10): 6762-6773).

As previously stated, the sequence specific reagent is preferably under the form of nucleic acids, such as under DNA or RNA form encoding a rare cutting endonuclease a subunit thereof, but they can also be part of conjugates involving polynucleotide(s) and polypeptide(s) such as so-called “ribonucleoproteins”. Such conjugates can be formed with reagents as Cas9 or Cpf1 (RNA-guided endonucleases) or Argonaute (DNA-guided endonucleases) as recently respectively described by Zetsche, B. et al. (Cpf1 Is a Single RNA-Guided Endonuclease of a Class 2 CRISPR-Cas System (2015) Cell 163(3): 759-771) and by Gao F. et al. (DNA-guided genome editing using the Natronobacterium gregoryi Argonaute (2016) Nature Biotech), which involve RNA or DNA guides that can be complexed with their respective nucleases.

“Exogenous sequence” refers to any nucleotide or polynucleotide sequence that was not initially present at a locus. This sequence may be homologous to, or a copy of, a genomic sequence, or be a foreign sequence introduced into the cell. By opposition “endogenous sequence” means a cell genomic sequence initially present at a locus. The exogenous sequence preferably comprises a sequence encoding for a polypeptide which expression confers a therapeutic advantage to the cell, especially resistance to proteasome inhibitors. This can be measured by contrast with respect to the same type of cells not having integrated this exogenous sequence at said locus. An endogenous sequence that is gene edited by the insertion of one nucleotide or polynucleotide for the expression of a modified or different polypeptide, becomes an exogenous coding sequence in the sense of the present invention.

The method of the present invention can be associated with other steps involving physical of genetic transformations, such as a viral transduction or transfection using nanoparticles, and also may be combined with other gene inactivation and/or transgene insertions.

### Large Scale Identification of Endogenous Genes Involved in Primary Cells' Drug Sensitivity

As part of the present invention is a general method to detect endogenous gene loci involved into the sensitivity of primary cells to a given drug, which provides a very useful—although not mandatory—tool to identify gene targets for producing resistant cells by gene editing. The loci identified through this method can be regarded as endogenous gene candidates for gene integration and/or inactivation for the purpose of the general invention disclosed in the present specification. This inventive screening method can be applied to any kind of drug compounds, but more particularly to proteasome inhibitors.

So far, endogenous genes involved into drug resistance had been identified in immortalized mutated cells lines, mostly by chemical or UV mutagenesis. However immortalized cell lines are not representative of primary cells, mainly because their transcriptional activities are completely different. Mutations in genes identified in cell lines can be indeed sometimes irrelevant since said genes are not necessarily expressed in primary cells and vice-versa.

Here, the inventors have managed to turn a method previously described by Shalem O, et al. (Genome-Scale CRISPR-Cas9 Knockout Screening in Human Cells (2014) Science, 343:84-87), so-called GECKO (Genome-scale CRISPR Knock-Out library), into a new method applicable to immune primary cells, especially T-cells.

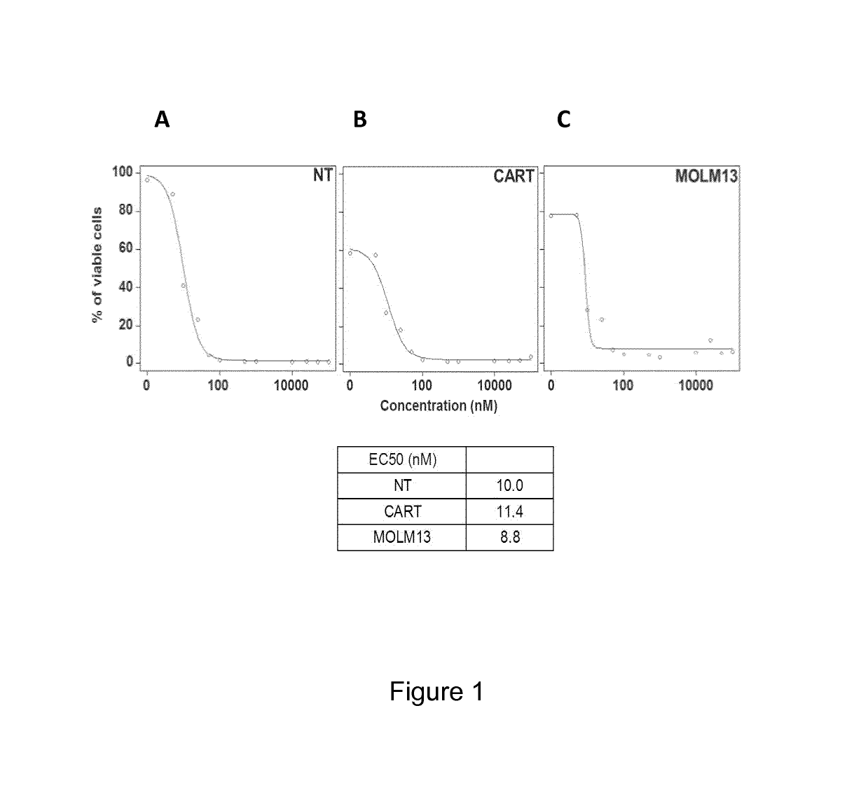

GECKO is originally based on the co-delivery of a library of guide RNA (sequence specific reagent) with a Cas9 (endonuclease that associates with the guide RNA to cleave DNA). This guide RNA library is generally delivered to the cells through expression of lentiviral vectors in which synthesized oligonucleotides encoding different guide RNAs (sgRNA) have been cloned. However, such libraries were limited and hardly applicable to primary T-cells for various reasons, among which the fact that (1) primary T-cells need an activation step (2) lentiviral delivery systems for CRISPR screening have low viral titer and (3) or required a cell line already expressing Cas9. Furthermore, the transduction of primary T-cells by lentiviral particles encoding the GECKO library was low when a standard transduction protocol was being used. The standard transduction protocol includes an initial activation of primary T-cells using CD3/CD8 coated magnetic beads (dynabeads or TransACT) followed by three days of expansion and lentiviral particles transduction. This protocol usually leads to a low or undetectable transduction efficiency as demonstrated using the puromycine resistance properties of transduced cells due to the presence of Puromycine resistance marker in the GECKO insertion cassette. As shown by the inventors, when a standard transduction protocol is being used as in Example 1, mock and GECKO transduced T-cells display similar puromycin resistance properties (FIG. 1). The inventors used this resistance to puromycin to monitor the transduction efficiency into the primary cells of the viral vectors encoding the variable sgRNAs along with Cas9.

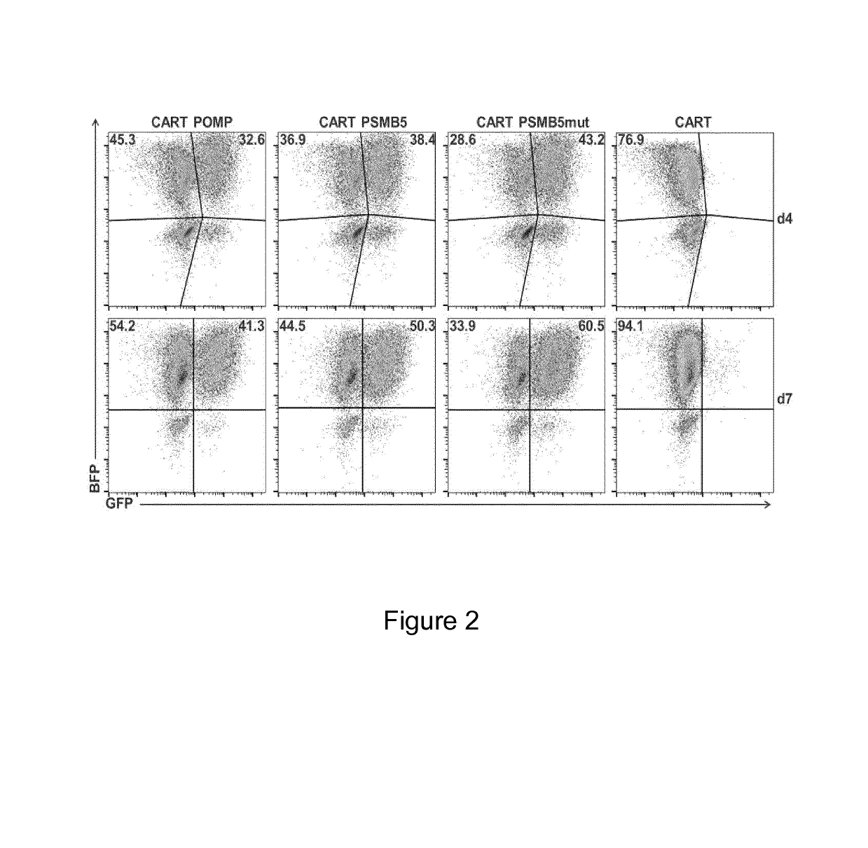

Surprisingly, the inventors found that simultaneous activation and transduction of the primary T-cells was significantly improving their ability to be transduced by the library of lentiviral particles. The data showed that transduction of primary T-cells on the day of their activation by CD3/CD8 coated magnetic beads, significantly improved the efficiency of their transduction by the library of GECKO lentiviral particles (FIG. 2). In this manner, genome-scale loss of sensitivity assays could be productively performed in primary immune cells.

Therefore, in one particular aspect, the present invention relates to a method for identifying loci conferring sensitivity of a primary immune cell to a drug, wherein said method comprises at least one or several of the following steps:
Providing primary immune cells;
Activating and transfecting said immune cells with a library of sequence specific endonuclease reagents within 24, preferably 20, more preferably 12 hours;
Selecting and amplifying the immune cells that have acquired resistance to the drug, preferably by culture in a culture medium comprising the drug, generally a LD50 dose of said drug;
Optionally: reproducing the genetic modification in the locus (loci) identified as conferring resistance to the drug, preferably by using another gene editing technique.

The library of sequence specific endonuclease reagents typically comprises more than 10, preferably 100, more preferably more than 1000 and even more preferably more than 2000 different sequence-specific reagents, active at various loci. In general, said specific endonuclease reagents are guide RNAs (gRNA) that associate with RNA guided-enzymes, such as guided endonucleases as for instance Cas9 or Cpf1. However, these libraries can also be formed of other sequence specific reagents, such as enzymes comprising transcription activator like effectors (TALE) or zing finger (ZF) binding domains that bind at different loci.

According to a preferred aspect of the invention, the genetic modifications into the endogenous loci of the primary cells are reproduced by transfection of mRNA encoding the sequence-specific reagent.

### General Method for Producing Engineered Immune Cells Resistant to Proteasome Inhibitors

The method described above has allowed identifying gene modifications, which could render primary cells resistant to proteasome inhibitors, especially by inactivating genes present at different loci by NHEJ. Thus, these loci are regarded by the inventors as favorable to introduce targeted gene modifications for producing engineered proteasome inhibitor resistant cells. A non-limited list of genes present at these loci is provided below. These genes are preferably inactivated by knocking down their endogenous sequences, either by introducing mutations or deletions using for instance a rare-cutting endonuclease, or by inserting an exogenous coding sequence that preferably expresses a gene product that also contributes to drug resistance.

As examples of proteins, which expression can be inactivated to improve resistance to proteasome inhibitors, are histone methyltransferases, such as EZH2, members of the BCL2 (Uniprot #P10415) protein family, preferably proteins sharing identity with such proteins or proteins interacting with same, such as BIM, BAX, BAK, BOK, BAD et BID.

EZH2 (Enhancer of zeste homolog 2) encodes histone-lysine N-methyltransferase enzyme (EC 2.1.1.43). This enzyme participates in DNA methylation by catalyzing the addition of methyl groups to histone H3. There is an advantage to inactivate the expression of this gene as part of the present invention because in addition to confer sensitivity to proteasome inhibitors, this gene has been found to be upregulated in multiple cancers. Preferably, EZH2 shares identity with human EZH2 of reference Uniprot #Q15910.

BIM is a pro-apoptotic member of the BCL-2 protein family that Interacts with other members of the BCL-2 protein family, including BCL2, BCL2L1/BCL-X(L), and MCL1, and act as an apoptotic activator. Its expression can be induced by nerve growth factor (NGF), as well as by the forkhead transcription factor FKHR-L1, which suggests a role in neuronal and lymphocyte apoptosis. This protein may function as an essential initiator of the apoptosis in thymocyte-negative selection. Nineteen alternatively spliced transcript variants of this gene have been reported. Preferably BIM shares identity with human BIM of reference Uniprot #043521. Alternative Names/Synonyms of BIM are B2L11; BAM; bcl-2 interacting mediator of cell death; bcl-2 interacting protein Bim; Bcl-2-like protein 11; bcl-2-related ovarian death agonist; Bcl2-interacting mediator of cell death; Bcl2-L-11; BCL2-like 11 (apoptosis facilitator); BCL2L11; BIM-alpha6; BIM-beta6; BIM-beta7; BimEL; BimL and BOD.

BIK (Bcl-2-interacting killer) are proteins that share identity and a critical BH3 domain with other death-promoting proteins, such as BID, BAK, BAD and BAX, which is required for its pro-apoptotic activity and for interaction with anti-apoptotic members of the BCL2 family. Since the activity of this protein is suppressed in the presence of survival-promoting proteins, it is suggested as a likely target for anti-apoptotic proteins. Preferably BIK shares identity with human BIK of reference Uniprot #Q13323. Alternative names are BIK, BIP1, BP4, NBK and BCL2 interacting killer.

BAX (Bcl-2-associated X protein), also known as bcl-2-like protein 4, form hetero- or homodimers and act as anti- or pro-apoptotic regulators that are involved in a wide variety of cellular activities. This protein forms a heterodimer with BCL2, and functions as an apoptotic activator. This protein is reported to interact with, and increase the opening of, the mitochondrial voltage-dependent anion channel (VDAC), which leads to the loss in membrane potential and the release of cytochrome c. The expression of this gene is regulated by the tumor suppressor P53 and has been shown to be involved in P53-mediated apoptosis. Preferably BAX shares identity with human BAX of reference Uniprot #Q07812 OR Q5ZPJ0.

BAK protein (Bcl-2 homologous antagonist/killer) is a protein that localizes in mitochondria, and that induces apoptosis. It interacts with and accelerates the opening of the mitochondrial voltage-dependent anion channel, which leads to a loss in membrane potential and the release of cytochrome c. This protein also interacts with the tumor suppressor P53 after exposure to cell stress. Preferably BAK shares identity with human BAK of reference Uniprot #Q16611. Alternative names are BAK1, BAK-LIKE, BCL2L7, CDN1 and BCL2 antagonist/killer 1.

Further examples are genes encoding PRKAA1, Cullin-3 and IPO4.

PRKAA1 (protein kinase AMP-activated catalytic subunit alpha 1) is a protein belonging to the ser/thr protein kinase family. It is the catalytic subunit of the 5′-prime-AMP-activated protein kinase (AMPK). AMPK is a cellular energy sensor conserved in all eukaryotic cells. The kinase activity of AMPK is activated by the stimuli that increase the cellular AMP/ATP ratio. AMPK regulates the activities of a number of key metabolic enzymes through phosphorylation. It protects cells from stresses that cause ATP depletion by switching off ATP-consuming biosynthetic pathways. Alternatively spliced transcript variants encoding distinct isoforms have been observed. Preferably PRKAA1 shares identity with human PRKAA1 preferably of reference Uniprot #Q13131.

Cullin-3 (CUL3), is a core component of multiple cullin-RING-based BCR (BTB-CUL3-RBX1) E3 ubiquitin-protein ligase complexes which mediate the ubiquitination and subsequent proteasomal degradation of target proteins. As a scaffold protein, it may contribute to catalysis through positioning of the substrate and the ubiquitin-conjugating enzyme. Preferably CUL3 shares identity with human CUL3 of reference Uniprot #Q13618.

IPO4 (Importin-4) is thought to mediate docking of the importin/substrate complex to the nuclear pore complex (NPC) through binding to nucleoporin and the complex is subsequently translocated through the pore by an energy requiring, Ran-dependent mechanism. Preferably IPO4 shares identity with human IPO4 of reference Uniprot#Q8TEX9. Alternative names are Imp4 or importin.

Other examples of proteins the inactivation of which were found to improve resistance to proteasome inhibitors, are: Ras-related protein Rab-6B (Rab6B), stress induced phosphoprotein 1 (STIP1), HECT domain-containing protein 2 (HECTD2), Coatomer subunit epsilon (COPE), Meiotic recombination protein DMC1(DMC1), NP002070, Putative exonuclease GOR (REXO1L1P), Surfeit locus protein 6 (SURF6), cAMP-dependent protein kinase catalytic subunit alpha (PRKACA) and cAMP-dependent protein kinase catalytic subunit gamma (PRKACG). These proteins have so far less known function than the previous ones. They respectively and preferably share identity with the human Rab6B protein of reference Uniprot #Q9NRW1, the human STIP1 protein of reference Uniprot #P31948, the human HECTD2 protein of reference Uniprot #Q5U5R9, the human COPE of reference Uniprot #014579, the human DMC1 protein of reference Uniprot #Q14565, the human REXO1L1P protein of reference Uniprot #Q8IX06, the human SURF6 protein of reference Uniprot #075683, the human PRKACA of reference Uniprot # P17612 and the human PRKACG protein of reference Uniprot #P22612.

According to one aspect, the present invention relates to a method for producing engineered proteasome inhibitor resistant cells by gene editing, comprises at least the steps of:
Performing gene editing into an endogenous gene with a sequence-specific reagent,
Selecting the cells that have acquired resistance to preferentially at least a LD50 dose of a proteasome inhibitor,
Expanding said cells.

This method can include further steps of conditioning, such as suspending the expanded cells with a pharmaceutical acceptable injectable buffer, and optionally of freezing the cells for a subsequent use.

The gene editing step performed into the at least one endogenous gene as per the present invention is preferably, but not necessarily, induced by a rare-cutting endonuclease so as to obtain more specific and stable gene editing. Said gene editing step can result into a modification of an endogenous locus, into the integration of an exogenous sequence or into the combined modification of the endogenous locus by the integration of an exogenous sequence, preferably a sequence coding conferring resistance to a proteasome inhibitor.

As indicated before, and as further illustrated in the examples, the invention may involve transfecting the cells with a library of sequence-specific reagents spanning a variety of endogenous genes sequences to inactivate those genes or integrate exogenous gene sequences, prior to selecting the cells that have acquired resistance to the proteasome inhibitor. Appropriate sequence-specific reagents can consist of a variety of guide RNA or DNA that associates with a guided endonuclease, such as Cas9, Cpf1 or Ago. “GECKO libraries” initially described by Shalem O., et al. (Genome-Scale CRISPR-Cas9 Knockout Screening in Human Cells (2014) Science, 343:84-87) can be used according to the invention. Such libraries are available under the form of viral vectors in which have been cloned a diversity of sequences encoding RNA guides targeting different gene sequences over the genome, preferably into open reading frames. The transduction of such viral library is made under controlled conditions of vector titration to reach a multiplicity of infection (MOI) of about 1 to obtain unique gene editing events. However, according to an even preferred embodiment, the MOI is increased to be superior or equals to 2, in order to obtain multiplex gene editing prompt to identify combinations of gene editing events conferring resistance to proteasome inhibitor. The inserted viral vectors can be retrieved in the cells that become resistant to the drugs by techniques known in the art, such as deep sequencing, single-cell PCR or digital PCR (Hindson, B. J. et al. (2012) “High-Throughput Droplet Digital PCR System for Absolute Quantitation of DNA Copy Number” Analytical Chemistry 83 (22): 8604-8610).

According to a preferred embodiment of the invention, the GECKO library is under the form of lentiviral vectors.

### Method for Making Cells Resistant to a Proteasome Inhibitor

As a general strategy to improve cell cancer therapy, proteasome inhibitors resistance is conferred to primary cells to protect them from the toxic effects of proteasome inhibitors treatment. The proteasome inhibitors resistance of primary cells also permits their enrichment in vitro or ex vivo, as primary cells which are proteasome inhibitors-resistant will survive and multiply relative to proteasome inhibitors sensitive cells. In particular, the present invention relates to a method of engineering proteasome inhibitors-resistant primary cells for combination therapy comprising:

(a) Providing a primary cell;

(b) Modifying the primary cell to confer proteasome inhibitors resistance to said primary cell;

(c) Expanding said engineered primary cell in the presence of a proteasome inhibitor.

The cells obtained according to the invention can be used as a medicament, especially in a combination therapy with proteasome inhibitors.

### Overexpression of a Proteasome Inhibitors Resistance Gene

According to one aspect of the invention, the immune cells can be made resistant to proteasome inhibitors by expression of exogenous coding sequences or over-expression of an endogenous gene, for instance by integration of an additional copy of said endogenous gene or integration of transcriptional enhancers (e.g. promoters, activators, stabilizing sequences . . . ).

The inventors have identified coding sequences, which expression improves resistance of primary cells to proteasome inhibitors, in particular those coding a protein selected from the list consisting of a proteasome subunit, a P-glycoprotein encoded by ATP-binding cassette sub-family B (ABCB) gene, a wnt glycoprotein, Interleukin-6 (IL-6), insulin-like growth factor-1 (IGF-1), insulin-like growth factor-1 receptor (IGF-1R), proteasomal beta5i subunit low molecular weight protein 7 (LMP7), a cluster of differentiation(CD) 52 (CD52), CD274, transcription factor 4 (TCF-4), nuclear factor (erythroid-derived 2)-like (NRF2), a transcription factor Yin Yang 1 (YY1), transcription elongation factor B1 (TCEB1), TCEB2, RING-box protein 1 (RBX1), anaphase promoting complex subunit 11 (ANAPC11), Von Hippel-Lindau tumor suppressor (VHL), a DNA damage-binding protein 1 (DDB1), a Src family kinase, preferably Lyn, a Phosphatidyl Inositol 3 kinase (PI3K), a Protein kinase B (AKT), a mechanistic target of rapamycin (mTOR), a heat shock protein (Hsp).

The expression of these coding sequences, whatever be the means or vector for their expression, can be combined with any other aspects of the present invention, in particular the stable gene editing methods described herein.

Coding sequences encoding proteasome subunits are preferably overexpressed to confer primary cells resistance to proteasome inhibitors

One example of such sequences encodes the proteasome subunit beta type-5 (PSMB5—Uniprot # P28074—SEQ ID NO.4) protein, which genomic sequence can share identity with SEQ ID NO.2

Another example of gene is the tripeptidyl peptidase II (TPPII) gene that encodes a large cytosolic oligopeptidase that sequentially removes tripeptides from the free N-terminus of short polypeptides. A study shown that overexpression of TPPII is sufficient to prevent accumulation of polyubiquitinated proteins and allows survival of cells at lethal concentrations of proteasome inhibitor (Wang, Kessler et al. 2000). Preferably TPPII shares identity with human TPPII of reference Uniprot #Q9V6K1

### Expression of Mutated Proteasome Subunits

In some embodiments, resistance to proteasome inhibitors can be conferred to the primary cells by expression of mutated forms of the sequences encoding some proteasome subunits, in particular PSMB5 and TPPII.

According to a preferred embodiment, mutant form of PSMB5 gene comprises at least one substituted amino acid at position Ala49, Ala50, Met45, or Cys52, more preferably at position Ala49 and or Ala50. In another particular embodiment, mutant form of PSMB5 comprises two mutated amino acids at position Ala49 and Ala50. In a particular embodiment, the alanine residue at position 49 is preferably replaced with a threonine residue, and the alanine residue at position 50 is preferably replaced with a valine residue.

The immune cells according to the invention thus generally comprise a mutated form of the PSMB5 gene sequence thereby expressing PSMB5 polypeptides that preferably comprise at least one mutation selected among Thr21Ala, Ala49Thr, Ala50Val, Cys52Phe, Met45Ile, Cys63Phe and Arg24Cys.

The above amino acid position refer to the canonical wild type polypeptide human sequence of PSBM5 referenced under P28074 in the Uniprot database herein referred to as SEQ ID NO.4).

Examples of such cells can express PSMB5 polypeptides comprising at least one of the following combined mutations:
Thr21Ala and Ala49Thr,
Thr21Ala and Ala50Val,
Thr21Ala and Cys52Phe,
Thr21Ala and Met45Ile,
Thr21Ala and Cys63Phe,
Thr21Ala and Arg24Cys,
Ala49Thr and Ala50Val,
Ala49Thr and Cys52Phe,
Ala49Thr and Met45lie
Ala49Thr and Cys63Phe,
Ala49Thr and Arg24Cys,
Ala50Val and Cys52Phe,
Ala50Val and Met45Ile,
Ala50Val and Cys63Phe,
Ala50Val and Arg24Cys,
Cys52Phe and Met45Ile,
Cys52Phe and Cys63Phe,
Cys52Phe and Arg24Cys,
Met45Ile and Cys63Phe,
Met45Ile and Arg24Cys,
Cys63Phe and Arg24Cys
Thr21Ala, Ala49Thr and Ala50Val,
Thr21Ala, Ala49Thr and Cys52Phe,
Thr21Ala, Ala49Thr and Met451Ile,
Thr21Ala, Ala49Thr and Cys63Phe,
Thr21Ala, Ala49Thr and Arg24Cys,
Thr21Ala, Ala50Val and Cys52Phe,
Thr21Ala, Ala50Val and Met451Ile,
Thr21Ala, Ala50Val and Cys63Phe,
Thr21Ala, Ala50Val and Arg24Cys,
Thr21Ala, Cys52Phe and Met45Ile,
Thr21Ala, Cys52Phe and Cys63Phe,
Thr21Ala, Cys52Phe and Arg24Cys,
Thr21Ala, Met45Ile and Cys63Phe,
Thr21Ala, Met45Ile and Arg24Cys,
Ala49Thr, Ala50Val and Cys52Phe,
Ala49Thr, Ala50Val and Met45Ile,
Ala49Thr, Ala50Val and Cys63Phe,
Ala49Thr, Ala50Val and Arg24Cys,
Ala49Thr, Cys52Phe and Met45Ile,
Ala49Thr, Cys52Phe and Cys63Phe,
Ala49Thr, Cys52Phe and Arg24Cys,
Ala49Thr, Met45Ile and Cys63Phe,
Ala49Thr, Met45Ile and Arg24Cys,
Ala50Val, Cys52Phe and Met45Ile,
Ala50Val, Cys52Phe and Cys63Phe,
Ala50Val, Cys52Phe and Arg24Cys,
Ala50Val, Met45Ile and Cys63Phe,
Ala50Val, Met45Ile and Arg24Cys,
Cys52Phe, Met45Ile and Cys63Phe,
Cys52Phe, Met45Ile and Arg24Cys,
Cys52Phe, Cys63Phe and Arg24Cys,
Met45Ile, Cys63Phe, and Arg24Cys,
Thr21Ala, Ala49Thr, Ala50Val and Cys52Phe,
Thr21Ala, Ala49Thr, Ala50Val and Met45Ile,
Thr21Ala, Ala49Thr, Ala50Val and Cys63Phe,
Thr21Ala, Ala49Thr, Ala50Val and Arg24Cys,
Thr21Ala, Ala50Val, Cys52Phe and Met45Ile,
Thr21Ala, Ala50Val, Cys52Phe and Cys63Phe,
Thr21Ala, Ala50Val, Cys52Phe and Arg24Cys,
Thr21Ala, Cys52Phe, Met45Ile and Cys63Phe,
Thr21Ala, Cys52Phe, Met45Ile and Arg24Cys,
Thr21Ala, Met45Ile, Cys63Phe and Arg24Cys,
Ala49Thr, Ala50Val, Cys52Phe and Met45Ile,
Ala49Thr, Ala50Val, Cys52Phe and Arg24Cys,
Ala49Thr, Ala50Val, Cys52Phe and Cys63Phe,
Ala49Thr, Ala50Val Cys63Phe and Arg24Cys,
Ala49Thr, Cys52Phe, Met451Ile and Cys63Phe,
Ala49Thr, Cys52Phe, Met451Ile and Arg24Cys,
Ala49Thr, Cys52Phe, Cys63Phe and Arg24Cys,
Ala49Thr, Met45Ile, Cys63Phe and Arg24Cys,
Ala50Val, Cys52Phe, Met451Ile and Cys63Phe,
Ala50Val, Cys52Phe, Met451Ile and Arg24Cys,
Ala50Val, Met45Ile, Cys63Phe and Arg24Cys,
Cys52Phe, Met45Ile, Cys63Phe and Arg24Cys,
Thr21Ala, Ala49Thr, Ala50Val, Cys52Phe and Met45Ile,
Thr21Ala, Ala49Thr, Ala50Val, Cys52Phe and Cys63Phe,
Thr21Ala, Ala49Thr, Ala50Val, Cys52Phe and Arg24Cys,
Thr21Ala, Ala49Thr, Ala50Val, Met45Ile and Cys63Phe,
Thr21Ala, Ala49Thr, Ala50Val, Met45Ile and Arg24Cys,
Thr21Ala, Ala49Thr, Ala50Val, Cys63Phe and Arg24Cys,
Thr21Ala, Ala50Val, Cys52Phe, Met45Ile and Cys63Phe,
Thr21Ala, Ala50Val, Cys52Phe, Met45Ile and Arg24Cys,
Thr21Ala, Ala50Val, Met45Ile, Arg24Cys and Cys63Phe,
Thr21Ala, Cys52Phe, Met45Ile, Cys63Phe and Arg24Cys,
Ala49Thr, Ala50Val, Cys52Phe, Met45Ile and Cys63Phe,
Ala49Thr, Ala50Val, Cys52Phe, Met45Ile and Arg24Cys,
Ala49Thr, Cys52Phe, Met45Ile, Cys63Phe and Arg24Cys,
Ala50Val, Cys52Phe, Met45Ile, Cys63Phe and Arg24Cys,
Thr21Ala, Ala49Thr, Ala50Val, Cys52Phe, Met451Ile and Cys63Phe,
Thr21Ala, Ala49Thr, Ala50Val, Cys52Phe, Met451Ile and Arg24Cys, and
Ala49Thr, Ala50Val, Cys52Phe, Met45Ile, Cys63Phe and Arg24Cys.

As previously described, the genetic modification step of the method can comprise a step of introduction into cells of an exogenous nucleic acid comprising at least a sequence encoding the proteasome inhibitors resistance gene and a portion of an endogenous gene such that homologous recombination occurs between the endogenous gene and the exogenous nucleic acid. In one embodiment, said endogenous gene can be the wild type “proteasome inhibitors resistance” gene, such that after homologous recombination, the wild type gene is replaced by the mutant form of the gene which confers resistance to the drug.

### Proteasome Inhibitors Resistant CAR T-Cells

According to one preferred embodiment, the proteasome inhibitors resistant primary cells of the invention are endowed with a chimeric antigen receptor (CAR) directed against at least one antigen expressed at the surface of a malignant or infected cell.

CARs are able to redirect immune cell specificity and reactivity toward a selected target exploiting the ligand-binding domain properties. Besides, CARs have successfully allowed T-cells to be redirected against antigens expressed at the surface of tumor cells from various malignancies including lymphomas and solid tumors (Sadelain M. et al. “The basic principles of chimeric antigen receptor design” (2013) Cancer Discov. 3(4):388-98).

Thus, in one embodiment, the method described herein further comprises a step of introducing a CAR into proteasome inhibitors resistant cells of the present invention.

CARs are synthetic receptors consisting of an extracellular ligand-binding domain that is associated with one or more signaling domains. In general, the extracellular ligand-binding domain of a CAR consists of an antigen-binding domain of a single-chain antibody (scFv), comprising the light and variable fragments of a monoclonal antibody joined by a flexible linker. The signaling domain is generally derived from the cytoplasmic region of the CD3, or from gamma chains of Fc receptor. The signaling domains are most often associated with co-stimulatory domain(s) from proteins, such as CD28, OX40, ICOS, CD137, CD8, CD28, OX40, ICOS, CD137, CD8, CD3, and 4-1BB (CD137) to enhance survival and increase proliferation of CAR modified T-cells.

In the present application, the term “signalling domain” refers to the portion of a protein which transduces the effector signal function signal and directs the cell to perform a specialized function.

Preferred examples of signal transducing domain for use in single or multi-chain CAR can be the cytoplasmic sequences of the Fc receptor or T cell receptor and co-receptors that act in concert to initiate signal transduction following antigen receptor engagement, as well as any derivate or variant of these sequences and any synthetic sequence that as the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d. According to particular embodiments, the signaling transducing domain of the multi-chain CAR can comprise the CD3zeta signaling domain, or the intracytoplasmic domain of the FcεRI beta or gamma chains.

The CAR of present invention may comprise a linker between said extracellular ligand-binding domain and said transmembrane domain. The term “linker” used herein generally means any oligo- or polypeptide that functions to link the transmembrane domain to the extracellular ligand-binding domain. In particular, linkers are used to provide more flexibility and accessibility for the extracellular ligand-binding domain. A linker may comprise up to 300 amino acids, preferably 10 to 100 amino acids and most preferably 25 to 50 amino acids. Linkers may be derived from all or part of naturally occurring molecules, such as from all or part of the extracellular region of CD8, CD4 or CD28, or from all or part of an antibody constant region. Alternatively the linker may be a synthetic sequence that corresponds to a naturally occurring linker sequence, or may be an entirely synthetic linker sequence. In a preferred embodiment, the linker is derived from CD8.

Ligand binding-domains can be any antigen receptor previously used, and referred to, with respect to single-chain CAR referred to in the literature, in particular scFv from monoclonal antibodies.

In preferred embodiments the extracellular ligand-binding domain is a scFv derived from an antibody directed against one of CS-1, CD38, BCMA, CD22, CLL-1, Hsp70 and CD123 antigen. Other extracellular ligand-binding domains can be useful for the treatment of malignant, especially those binding a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-I IRa, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-AI, legumain, HPV E6, E7, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin BI, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

Other binding domain than scFv can also be used for predefined targeting of lymphocytes, such as camelid single-domain antibody fragments or receptor ligands like a vascular endothelial growth factor polypeptide, an integrin-binding peptide, heregulin or an IL-13 mutein, antibody binding domains, antibody hypervariable loops or CDRs as non-limiting examples.

The proteasome inhibitors resistant primary cells of the present invention may be engineered to express a CAR either under a single-chain form (scCAR) or under a multi-chain form (mcCAR).

Multi-chain Chimeric Antigen Receptor are formed by multiple polypeptides to allow normal juxtamembrane position of all relevant signaling domains as described in WO2013176916. According to such architectures, ligands binding domains and signaling domains are born on separate polypeptides. The different polypeptides are anchored into the membrane in a close proximity allowing interactions with each other. In such architectures, the signaling and co-stimulatory domains can be in juxtamembrane positions (i.e. adjacent to the cell membrane on the internal side of it), which is deemed to allow improved function of co-stimulatory domains. The multi-subunit architecture also offers more flexibility and possibilities of designing CARs with more control on T-cell activation. For instance, it is possible to include several extracellular antigen recognition domains having different specificity to obtain a multi-specific CAR architecture. It is also possible to control the relative ratio between the different subunits into the multi-chain CAR.

The assembly of the different chains as part of a single multi-chain CAR is made possible, for instance, by using the different alpha, beta and gamma chains of the high affinity receptor for IgE (FcεRI) (Metzger, Alcaraz et al. 1986) to which are fused the signaling and co-stimulatory domains. The gamma chain comprises a transmembrane region and cytoplasmic tail containing one immunoreceptor tyrosine-based activation motif (ITAM) (Cambier 1995).

The multi-chain CAR can comprise several extracellular ligand-binding domains, to simultaneously bind different elements in target thereby augmenting immune cell activation and function. In one embodiment, the extracellular ligand-binding domains can be placed in tandem on the same transmembrane polypeptide, and optionally can be separated by a linker. In another embodiment, said different extracellular ligand-binding domains can be placed on different transmembrane polypeptides composing the multi-chain CAR.

The signal transducing domain or intracellular signaling domain of the multi-chain CAR(s) of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the multi-chain CAR is expressed. For example, the effector function of a T cell can be a cytolytic activity or helper activity including the secretion of cytokines.

Accordingly, a CAR expressed by the engineered cell according to the invention can be a multi-chain chimeric antigen receptor particularly adapted to the production and expansion of engineered cells of the present invention. Such multi-chain CARs comprise at least two of the following components:

a) One polypeptide comprising the transmembrane domain of FcεRI alpha chain and an extracellular ligand-binding domain,

b) One polypeptide comprising a part of N- and C-terminal cytoplasmic tail and the transmembrane domain of FcεRI beta chain and/or

c) At least two polypeptides comprising each a part of intracytoplasmic tail and the transmembrane domain of FcεRI gamma chain, whereby different polypeptides multimerize together spontaneously to form dimeric, trimeric or tetrameric CAR.

According to particular embodiments, the signal transduction domain of multi-chain CARs of the present invention comprises a co-stimulatory signal molecule. A co-stimulatory molecule is a cell surface molecule other than an antigen receptor or their ligands that is required for an efficient immune response.

Also the present specification broadly relates to methods of producing stably engineered proteasome inhibitor-resistant cells suitable for their use in immunotherapy in combination with a proteasome inhibitor, comprising:

(a) Providing cells;

(b) introducing at least one gene encoding a CAR using a lentiviral vector, and

(c) Editing the genome of cells by carrying out one of the following events: inserting, mutating, deleting, substituting at least one coding and/or non-coding sequence, and a combination thereof, to confer resistance to a proteasome inhibitor, and by transducing optionally selectivity against a specific target molecule using at least one specific endonuclease targeting said coding and/or non-coding sequence, and optionally

(a) growing cells in the presence of an effective therapeutic dose of a proteasome inhibitor, preferably a dose ≤0.1 nM in vitro or 0.01 mg/m² in vivo.

### Method of Engineering “Off-the-Shelf’ and Proteasome Inhibitors Resistant Immune Cells

According to a particular aspect, the present invention relates to a method of making “off-the-shelf” immune cells, especially T-cells or derivatives thereof, resistant to proteasome inhibitors, especially suitable for combination therapy.

According to the present invention, engraftment of allogeneic T-cells is possible by inactivating at least one gene encoding a TCR component, for instance by introducing gene modifications into TCRα gene and/or TCRβ gene(s) as described for instance in WO2013176915. TCR inactivation in allogeneic T-cells reduces Graft-versus-host disease (GvHD). By inactivating a gene it is generally meant that the expression of the gene is significantly reduced or that the gene of interest is not expressed into a functional protein form.

In particular embodiments, the genetic modification relies on a stable non-functional mutation introduced into the coding sequence by a rare cutting endonuclease. The nucleic acid strand breaks caused by the rare-cutting endonuclease are commonly repaired through the distinct mechanisms of homologous recombination or nonhomologous end joining (NHEJ). However, NHEJ is an imperfect repair process that often results in changes to the DNA sequence at the site of the cleavage. Mechanisms involve rejoining of what remains of the two DNA ends through direct re-ligation (Critchlow and Jackson 1998) or via the so-called microhomology-mediated end joining (Betts, Brenchley et al. 2003; Ma, Kim et al. 2003). Repair via non-homologous end joining (NHEJ) often results in small insertions or deletions and can be used for the creation of specific gene knockouts. Said modification may be a substitution, deletion, or addition of at least one nucleotide. Cells in which a cleavage-induced mutagenesis event, i.e. a mutagenesis event consecutive to an NHEJ event, has occurred can be identified and/or selected by well-known method in the art. In a particular embodiment, the step of inactivating at least a gene encoding a component of the T-cell receptor (TCR) into the cells of each individual sample comprises introducing into the cell a rare-cutting endonuclease able to disrupt at least one gene encoding a component of the T-cell receptor (TCR). In a more particular embodiment, said cells of each individual sample are transformed with nucleic acid encoding a rare-cutting endonuclease capable of disrupting at least one gene encoding a component of the T-cell receptor (TCR), and said rare-cutting endonuclease is expressed into said cells.

Accordingly, the present invention is more particularly drawn to proteasome inhibitor (PI) resistant non allo-reactive primary cells, comprising:
an edited endogenous sequence comprising an inactivation of a T cell receptor gene, preferably of a TCRα and/or TCRβ gene,
an exogenous polynucleotidique sequence coding a chimeric antigen receptor (CAR) and/or a TCR, specific for a molecule expressed at the surface of a pathological cell,
at least one additional edited endogenous sequence with an insertion and/or a deletion conferring resistance to a proteasome inhibitor as compared to non-edited cells.

### Enhancing Persistence of the Immune Cells According to the Invention In-Vivo

By “enhancing persistence” is meant extending the survival of the immune cells in terms of life span, especially once the engineered immune cells are injected into the patient. For instance, persistence is enhanced, if the mean survival of the modified cells is significantly longer than that of non-modified cells, by at least 10%, preferably 20%, more preferably 30%, even more preferably 50%.

This is especially relevant when the immune cells are allogeneic. This may be done by creating a local immune protection by introducing coding sequences that ectopically express and/or secrete immunosuppressive polypeptides at, or through, the cell membrane. A various panel of such polypeptides in particular antagonists of immune checkpoints, immunosuppressive peptides derived from viral envelope or NKG2D ligand can enhance persistence and/or an engraftment of allogeneic immune cells into patients.

According to one aspect of the present method, an exogenous sequence is introduced into the immune cells resistant to proteasome inhibitors of the present invention to enhance persistence of the immune cells, especially in-vivo persistence in a tumor environment.

According to one embodiment, said exogenous coding sequence encodes an immunosuppressive polypeptide such as a ligand of Cytotoxic T-Lymphocyte Antigen 4 (CTLA-4 also known as CD152, GenBank accession number AF414120.1). Said ligand polypeptide is preferably an anti-CTLA-4 immunoglobulin, such as CTLA-4a Ig and CTLA-4b Ig or a functional variant thereof.

According to a further embodiment, the immunosuppressive polypeptide to be encoded by said exogenous coding sequence is an antagonist of PD1, such as PD-L1 (other names: CD274, Programmed cell death 1 ligand; ref. UniProt # Q9NZQ7), which encodes a type I transmembrane protein of 290 amino acids consisting of a Ig V-like domain, a Ig C-like domain, a hydrophobic transmembrane domain and a cytoplasmic tail of 30 amino acids. Such membrane-bound form of PD-L1 ligand is meant in the present invention under a native form (wild-type) or under a truncated form such as, for instance, by removing the intracellular domain, or with one or more mutation(s) (Wang S et al., 2003, J Exp Med. 2003; 197(9): 1083-1091). Of note, PD1 is not considered as being a membrane-bound form of PD-L1 ligand according to the present invention. According to another embodiment, said immunosuppressive polypeptide is under a secreted form. Such recombinant secreted PD-L1 (or soluble PD-L1) may be generated by fusing the extracellular domain of PD-L1 to the Fc portion of an immunoglobulin (Haile S T et al., 2014, Cancer Immunol. Res. 2(7): 610-615; Song M Y et al., 2015, Gut. 64(2):260-71). This recombinant PD-L1 can neutralize PD-1 and abrogate PD-1-mediated T-cell inhibition. PD-L1 ligand may be co-expressed with CTLA4 Ig for an even enhanced persistence of both.

According to another embodiment, the exogenous sequence encodes a polypeptide comprising a viral env immusuppressive domain (ISU), which is derived for instance from HIV-1, HIV-2, SIV, MoMuLV, HTLV-I, -II, MPMV, SRV-1, Syncitin 1 or 2, HERV-K or FELV.

The following Table 1 shows variants of ISU domains from diverse viruses which can be expressed within the present invention.

TABLE 1
ISU domain variants from diverse viruses
ISU Amino acids sequences

| Amino acid positions | | | | | | | | | | | | | | Virus origin |
|---|---|---|---|---|---|---|---|---|---|---|---|---|---|---|
| 1 | 2 | 3 | 4 | 5 | 6 | 7 | 8 | 9 | 10 | 11 | 12 | 13 | 14 | Origin |
| L | Q | A | R | I/V | L | A | V | E | R | Y | L | K/R/Q | D | HIV-1 |
| L | Q | A | R | V | T | A | I | E | K | Y | L | K/A/Q | D/H | HIV-2 |
| L | Q | A | R | L | L | A | V | E | R | Y | L | K | D | SIV |
| L | Q | N | R | R | G | L | D | L | L | F | L | K | E | MoMuLV |
| A | Q | N | R | R | G | L | D | L | L | F | W | E | Q | HTLV-I, -II |
| L | Q | N | R | R | G | L | D | L | L | T | A | E | Q | MPMV, SRV-1 |
| L | Q | N | R | R | A | L | D | L | L | T | A | E | R | Syncitin 1 |
| L | Q | N | R | R | G | L | D | M | L | T | A | A | Q | Syncitin 2 |
| L | A | N | Q | I | N | D | L | R | Q | T | V | I | W | HERV-K |
| L | Q | N | R | R | G | L | D | I | L | F | L | Q | E | FELV |

According to another embodiment, the exogenous sequence encodes a FP polypeptide such as gp41. The following Table 2 represents several FP polypeptide from natural and artificial origins.

TABLE 2
Aminoacid sequences of FP polypeptide
from natural and artificial origins
FP Amino acids sequences

| Amino acid positions | | | | | | | | | |
|---|---|---|---|---|---|---|---|---|---|
| 1 | 2 | 3 | 4 | 5 | 6 | 7 | 8 | 9 | Origin |
| G | A | L | F | L | G | F | L | G | HIV-1 gp41 |
| A | G | F | G | L | L | L | G | F | Synthetic |
| A | G | L | F | L | G | F | L | G | Synthetic |

According to another embodiment, the exogenous sequence encodes a non-human MHC homolog, especially a viral MHC homolog, or a chimeric β2m polypeptide such as described by Margalit A. et al. ((2003) “Chimeric β2 microglobulin/CD3, polypeptides expressed in T cells convert MHC class I peptide ligands into T cell activation receptors: a potential tool for specific targeting of pathogenic CD8+ T cells” Int. Immunol. 15 (11): 1379-1387).

According to one embodiment, the exogenous sequence encodes NKG2D ligand. Some viruses such as cytomegaloviruses have acquired mechanisms to avoid NK cell mediate immune surveillance and interfere with the NKG2D pathway by secreting a protein able to bind NKG2D ligands and prevent their surface expression (Welte, S. A et al. (2003) “Selective intracellular retention of virally induced NKG2D ligands by the human cytomegalovirus UL16 glycoprotein”. Eur. J. Immunol., 33, 194-203). In tumors cells, some mechanisms have evolved to evade NKG2D response by secreting NKG2D ligands such as ULBP2, MICB or MICA (Salih H R, Antropius H, Gieseke F, Lutz S Z, Kanz L, et al. (2003) Functional expression and release of ligands for the activating immunoreceptor NKG2D in leukemia. Blood 102: 1389-1396)

According to one embodiment, the exogenous sequence encodes a cytokine receptor, such as an IL-12 receptor. IL-12 is a well-known activator of immune cells activation (Curtis J. H. (2008) “IL-12 Produced by Dendritic Cells Augments CD8+ T Cell Activation through the Production of the Chemokines CCL1 and CCL171”. The Journal of Immunology. 181 (12): 8576-8584.

According to one embodiment the exogenous sequence encodes an antibody that is directed against inhibitory peptides or proteins. Said antibody is preferably be secreted under soluble form by the immune cells. Nanobodies from shark and camels are advantageous in this respect, as they are structured as single chain antibodies (Muyldermans S. (2013) “Nanobodies: Natural Single-Domain Antibodies” Annual Review of Biochemistry 82: 775-797). Same are also deemed more easily to fuse with secretion signal polypeptides and with soluble hydrophilic domains.

The different aspects developed above to enhance persistence of the cells are particularly preferred, when the exogenous coding sequence is introduced by disrupting an endogenous gene encoding 132m or another MHC component, as detailed for instance in WO2016142532.

### Enhancing the Therapeutic Activity of Immune Cells

According to one aspect of the present method, an exogenous sequence can be introduced in the immune cells resistant to proteasome inhibitors according to the invention to encode a molecule that enhances the therapeutic activity of the immune cells.

By “enhancing the therapeutic activity” is meant that the immune cells, or population of cells, engineered according to the present invention, become more aggressive than non-engineered cells or population of cells with respect to a selected type of target cells. Said target cells consists of a defined type of cells, or population of cells, preferably characterized by common surface marker(s). In the present specification, “therapeutic potential” reflects the therapeutic activity, as measured through in-vitro experiments. In general sensitive cancer cell lines, such as Daudi cells, are used to assess whether the immune cells are more or less active towards said cells by performing cell lysis or growth reduction measurements. This can also be assessed by measuring levels of degranulation of immune cells or chemokines and cytokines production. Experiments can also be performed in mice with injection of tumor cells, and by monitoring the resulting tumor expansion. Enhancement of activity is deemed significant when the number of developing cells in these experiments is reduced by the immune cells by more than 10%, preferably more than 20%, more preferably more than 30%, even more preferably by more than 50%.

According to one aspect of the invention, said exogenous sequence encodes a chemokine or a cytokine, such as IL-12. It is particularly advantageous to express IL-12 as this cytokine is extensively referred to in the literature as promoting immune cell activation (Colombo M. P. et al. (2002) “lnterleukin-12 in anti-tumor immunity and immunotherapy” Cytokine Growth Factor Rev. 13(2):155-68).

According to a preferred aspect of the invention the exogenous coding sequence encodes or promote secreted factors that act on other populations of immune cells, such as T-regulatory cells, to alleviate their inhibitory effect on said immune cells.

According to one aspect of the invention, said exogenous sequence encodes an inhibitor of regulatory T-cell activity is a polypeptide inhibitor of forkhead/winged helix transcription factor 3 (FoxP3), and more preferably is a cell-penetrating peptide inhibitor of FoxP3, such as that referred as P60 (Casares N. et al. (2010) “A peptide inhibitor of FoxP3 impairs regulatory T cell activity and improves vaccine efficacy in mice.” J Immunol 185(9):5150-9).

By “inhibitor of regulatory T-cells activity” is meant a molecule or precursor of said molecule secreted by the T-cells and which allow T-cells to escape the down regulation activity exercised by the regulatory T-cells thereon. In general, such inhibitor of regulatory T-cell activity has the effect of reducing FoxP3 transcriptional activity in said cells.

According to one aspect of the invention, said exogenous sequence encodes a secreted inhibitor of Tumor Associated Macrophages (TAM), such as a CCR2/CCL2 neutralization agent. Tumor-associated macrophages (TAMs) are critical modulators of the tumor microenvironment. Clinicopathological studies have suggested that TAM accumulation in tumors correlates with a poor clinical outcome. Consistent with that evidence, experimental and animal studies have supported the notion that TAMs can provide a favorable microenvironment to promote tumor development and progression. (Theerawut C. et al. (2014) “Tumor-Associated Macrophages as Major Players in the Tumor Microenvironment” Cancers (Basel) 6(3): 1670-1690). Chemokine ligand 2 (CCL2), also called monocyte chemoattractant protein 1 (MCP1—NCBI NP_002973.1), is a small cytokine that belongs to the CC chemokine family, secreted by macrophages, that produces chemoattraction on monocytes, lymphocytes and basophils. CCR2 (C—C chemokine receptor type 2—NCBI NP_001116513.2), is the receptor of CCL2.

### Improving the Efficiency of Gene Targeted Insertion in Primary Immune Cells Using AAV Vectors

Gene targeted insertion into human primary cells as per the present invention can be efficiently performed by using AAV vectors, especially vectors from the AAV6 family. Transduction of AAV vectors in human primary immune cells can be made easily in conjunction with the expression of sequence specific endonuclease reagents, such as TALE endonuclease.

According to one aspect, sequence specific endonuclease reagents can be introduced into the cells by transfection, more preferably by electroporation of mRNA encoding said sequence specific endonuclease reagent(s).

Still according to this aspect, the invention more particularly provides a method of insertion of an exogenous nucleic acid sequence into an endogenous polynucleotide sequence in a cell, comprising at least the steps of:
transducing into said cell an AAV vector comprising said exogenous nucleic acid sequence and sequences homologous to the targeted endogenous DNA sequence, and
Inducing the expression of a sequence specific endonuclease reagent to cleave said endogenous sequence at the locus of insertion.

The obtained insertion of the exogenous nucleic acid sequence may result into the introduction of genetic material, correction or replacement of the endogenous sequence, more preferably “in frame” with respect to the endogenous gene sequences at that locus.

According to another aspect of the invention, from 10⁵ to 10⁷, preferably from 10⁶ to 10⁷, more preferably about 5·10⁶ viral genomes viral genomes are transduced per cell.

According to another aspect of the invention, the cells can be treated with proteasome inhibitors, such as Bortezomib to further help homologous recombination.

As one object of the present invention, the AAV vector used in the method can comprise an exogenous coding sequence that is promoterless, said coding sequence being any of those referred to in this specification.

As one object of the present invention, the AAV vector used in the method can comprise a 2A peptide cleavage site followed by the cDNA (minus the start codon) forming the exogenous coding sequence.

As one object of the present invention, said AAV vector comprises an exogenous sequence coding for a chimeric antigen receptor, especially an anti-CD19 CAR, an anti-CD22 CAR, an anti-CD123 CAR, an anti-CS1 CAR, an anti-CCL1 CAR, an anti-HSP70 CAR, an anti-GD3 CAR or an anti-ROR1 CAR.

The invention thus encompasses any AAV vectors designed to perform the method herein described, especially vectors comprising a sequence homologous to a locus of insertion located in any of the endogenous gene responsive to T-cell activation referred to in Table 5.

Many other vectors known in the art, such as plasmids, episomal vectors, linear DNA matrices, etc. . . . can also be used following the teachings to the present invention.

As stated before, the DNA vector used according to the invention preferably comprises: (1) said exogenous nucleic acid comprising the exogenous coding sequence to be inserted by homologous recombination, and (2) a sequence encoding the sequence specific endonuclease reagent that promotes said insertion. According to a more preferred aspect, said exogenous nucleic acid under (1) does not comprise any promoter sequence, whereas the sequence under (2) has its own promoter. According to an even more preferred aspect, the nucleic acid under (1) comprises an Internal Ribosome Entry Site (IRES) or “self-cleaving” 2A peptides, such as T2A, P2A, E2A or F2A, so that the endogenous gene where the exogenous coding sequence is inserted becomes multi-cistronic. The IRES of 2A Peptide can precede or follow said exogenous coding sequence.

### Gene Targeted Integration in Immune Cells Under Transcriptional Control of Endogenous Promoters

The present invention, in one of its main aspects, is taking advantage of the endogenous transcriptional activity of the immune cells to express the coding sequences that confer resistance to proteasome inhibitors and the other coding sequences referred to in the previous sections improving the therapeutic potential of the immune cells.

The invention provides with several embodiments based on the profile of transcriptional activity of the endogenous promoters and on a selection of promoter loci useful to carry out the invention. Preferred loci are those, which transcription activity is generally high upon immune cell activation, especially in response to CAR activation (CAR-sensitive promoters) when the cells are endowed with CARs.

The inventors have established a first list of endogenous genes (Table 5) which have been found to be particularly appropriate for applying the targeted gene recombination as per the present invention. To draw this list, they have come across several transcriptome murine databases, in particular that from the Immunological Genome Project Consortium referred to in Best J. A. et al. (2013) “Transcriptional insights into the CD8(+) T cell response to infection and memory T cell formation” Nat. Immunol.. 14(4):404-12., which allows comparing transcription levels of various genes upon T-cell activation, in response to ovalbumin antigens. Also, because very few data is available with respect to human T-cell activation, they had to make some extrapolations and analysis from these data and compare with the human situation by studying available literature related to the human genes. The selected loci of Table 6 are particularly relevant for the insertion of coding sequences, which expression confers resistance to proteasome inhibitors. However, the inventors have designed different strategies based on the expression profiles of the promoters present at said loci (Tables 6 to 9).

Gene Targeted Insertion Under Control of Endogenous Promoters that are Steadily Active During Immune Cell Activation

A selection of endogenous gene loci is listed in Table 7, which are transcriptionally and steadily active during immune cell activation.

By “immune cell activation” is meant production of an immune response as per the mechanisms generally described and commonly established in the literature for a given type of immune cells. With respect to T-cell, for instance, T-cell activation is generally characterized by one of the changes consisting of cell surface expression by production of a variety of proteins, including CD69, CD71 and CD25 (also a marker for Treg cells), and HLA-DR (a marker of human T cell activation), release of perforin, granzymes and granulysin (degranulation), or production of cytokine effectors IFN-γ, TNF and LT-alpha.

According to a preferred embodiment of the invention, the transcriptional activity of the endogenous gene is up-regulated in the immune cell, especially in response to an activation by a CAR. The CAR can be independently expressed in the immune cell. By “independently expressed” is meant that the CAR can be transcribed in the immune cell from an exogenous expression cassette introduced, for instance, using a retroviral vector, such as a lentiviral vector.

The promoters present at the loci of Table 6 are deemed most appropriate for obtaining primary immune cells resistance to proteasome inhibitors as long as the immune cell remains in an active stage.

Accordingly the method of the present invention provides with the step of performing gene targeted insertion under control of an endogenous promoter that is steadily active during immune cell activation, preferably from of an endogenous gene selected from CD3G, Rn28s1, Rn18s, Rn7sk, Actg1, β2m, Rpl18a, Pabpc1, Gapdh, Rpl17, Rpl19, Rplp0, Cfl1 and Pfn1.

By “steadily active” means that the transcriptional activity observed for these promoters in the primary immune cell is not affected by a negative regulation upon the activation of the immune cell.

As reported elsewhere (Acuto, O. (2008) “Tailoring T-cell receptor signals by proximal negative feedback mechanisms”. Nature Reviews Immunology 8:699-712), the promoters present at the TCR locus are subjected to different negative feedback mechanisms upon TCR engagement and thus may not be steadily active or up regulated during for the method of the present invention. The present invention has been designed to some extend to avoid using the TCR locus as a possible insertion site for exogenous coding sequences to be expressed during T-cell activation. Therefore, according to one aspect of the invention, the targeted insertion of the exogenous coding sequence is not performed at a TCRalpha or TCRbeta gene locus.

In addition to the coding sequences conferring resistance to proteasome inhibitors, examples of other exogenous coding sequence that can be advantageously introduced at such loci under the control of steadily active endogenous promoters, are those encoding or positively regulating the production of a cytokine, a chemokine receptor, a molecule conferring resistance to a drug, a co-stimulation ligand, such as 4-1 BRL and OX40L, or of a secreted antibody.

Gene Integration Under Endogenous Promoters that are Dependent from Immune Cell Activation

As stated before, the method of the present invention provides with the step of performing gene targeted insertion under control of an endogenous promoter, which transcriptional activity is preferably up-regulated upon immune cell activation, either transiently or over more than 10 days.

Said endogenous gene whose transcriptional activity is up regulated are particularly appropriate for the integration of the coding sequences conferring resistance to proteasome inhibitors and also of other exogenous sequences, such as those encoding cytokine(s), immunogenic peptide(s), or a secreted antibody, such as an anti-IDO1, anti-IL10, anti-PD1, anti-PDL1, anti-lL6 or anti-PGE2 antibody.

These endogenous promoters are particularly advantageous because they induce cell resistance to proteasome inhibitors when the immune cells are the most active, but over a limited period of time. This embodiment can be regarded as safer, since the cells get eliminated after said period of time, which is generally less than 20 days, preferably less than 15 days, even more preferably less than 10 days.

Depending on the level of resistance desired, the endogenous gene is selected for a weak or a strong up-regulation. The exogenous coding sequence introduced into said endogenous gene whose transcriptional activity is weakly up regulated, can be advantageously a constituent of an inhibitory CAR, or of an apoptotic CAR, which expression level has generally to remain lower than that of a positive CAR. Such combination of CAR expression, for instance one transduced with a viral vector and the other introduced according to the invention, can greatly improve the specificity or safety of CAR immune cells Some endogenous promoters are transiently up-regulated, sometimes over less than 12 hours upon immune cell activation, such as those selected from the endogenous gene loci Spata6, Itga6, Rcbtb2, Cdldl, St8sia4, Itgae and Fam214a (Table 8. Other endogenous promoters are up-regulated over less than 24 hours upon immune cell activation, such as those selected from the endogenous gene loci IL3, IL2, Ccl4, IL21, Gp49a, Nr4a3, Lilrb4, Cd200, Cdkn1a, Gzmc, Nr4a2, Cish, Ccr8, Lad1 and Crabp2 (Table 9) and others over more than 24 hours, more generally over more than 10 days, upon immune cell activation. Such as those selected from Gzmb, Tbx21, Plek, Chek1, Slamf7, Zbtb32, Tigit, Lag3, Gzma, Wee1, IL12rb2, Eea1 and Dt1 (Table 9).

### Gene Targeted Insertion and Expression of Genes Conferring Resistance to Proteasome Inhibitors in Hematopoietic Stem Cells (HSCs)

One aspect of the present invention more particularly concerns the insertion of transgenes into hematopoietic stem cells (HSCs).

Hematopoietic stem cells (HSCs) are multipotent, self-renewing progenitor cells from which all differentiated blood cell types arise during the process of hematopoiesis. These cells include lymphocytes, granulocytes, and macrophages of the immune system as well as circulating erythrocytes and platelets. Classically, HSCs are thought to differentiate into two lineage-restricted, lymphoid and myelo-erythroid, oligopotent progenitor cells. The mechanisms controlling HSC self-renewal and differentiation are thought to be influenced by a diverse set of cytokines, chemokines, receptors, and intracellular signaling molecules. Differentiation of HSCs is regulated, in part, by growth factors and cytokines including colony-stimulating factors (CSFs) and interleukins (ILs) that activate intracellular signaling pathways. The factors depicted below are known to influence HSC multipotency, proliferation, and lineage commitment. HSCs and their differentiated progeny can be identified by the expression of specific cell surface lineage markers such as cluster of differentiation (CD) proteins and cytokine receptors into hematopoietic stem cells.

Gene therapy using HSCs has enormous potential to treat diseases of the hematopoietic system including immune diseases. In this approach, HSCs are collected from a patient, gene-modified ex-vivo using integrating retroviral vectors, and then infused into a patient. HSCs are commonly harvested from the peripheral blood after mobilization (patients receive recombinant human granulocyte-colony stimulating factor (G-CSF)). The patient's peripheral blood is collected and enriched for HSCs using the CD34+ marker. HSCs are then cultured ex vivo and exposed to viral vectors. The ex vivo culture period varies from 1 to 4 days. Prior to the infusion of gene-modified HSCs, patients may be treated with chemotherapy agents, especially proteasome inhibitors or irradiation to help enhance the engraftment efficiency. Gene-modified HSCs are re-infused into the patient intravenously. The cells migrate into the bone marrow before finally residing in the sinusoids and perivascular tissue. Both homing and hematopoiesis are integral aspects of engraftment. Cells that have reached the stem cell niche through homing will begin producing mature myeloid and lymphoid cells from each blood lineage. Hematopoiesis continues through the action of long-term HSCs, which are capable of self-renewal for life-long generation of the patient's mature blood cells, in particular the production of common lymphoid progenitor cells, such as T cells and NK cells, which are key immune cells for eliminating infected and malignant cells.

The present invention provides with performing gene targeted insertion in HSCs to introduce exogenous coding sequences conferring resistance to proteasome inhibitors under the control of endogenous promoters, especially endogenous promoters of genes that are specifically activated into cells of a particular hematopoietic lineage or at particular differentiation stage, preferably at a late stage of differentiation. The HSCs can be transduced with a polynucleotide vector (donor template), such as an AAV vector, during an ex-vivo treatment as referred to in the previous paragraph, whereas a sequence specific nuclease reagent is expressed as to promote the insertion of the coding sequences at the selected locus. The resulting engineered HSCs can be then engrafted into a patient in need thereof for a long term in-vivo production of engineered immune cells that will comprise said exogenous coding sequences. Depending on the activity of the selected endogenous promoter, the coding sequences will be selectively expressed in certain lineages or in response to the local environment of the immune cells in-vivo, thereby providing adoptive immunotherapy.

According to one preferred aspect of the invention, the exogenous coding sequences are placed under the control of promoters of a gene, which transcriptional activity is specifically induced in common lymphoid progenitor cells, such as CD34, CD43, Flt-3/Flk-2, IL-7 R alpha/CD127 and Neprilysin/CD10.

More preferably, the exogenous coding sequences are placed under the control of promoters of a gene, which transcriptional activity is specifically induced in NK cells, such as CD161, CD229/SLAMF3, CD96, DNAM-1/CD226, Fc gamma RII/CD32, Fc gamma RII/RIII (CD32/CD16), Fc gamma RIII (CD16), IL-2 R beta, Integrin alpha 2/CD49b, KIR/CD158, NCAM-1/CD56, NKG2A/CD159a, NKG2C/CD159c, NKG2D/CD314, NKp30/NCR3, NKp44/NCR2, NKp46/NCR1, NKp80/KLRF1, Siglec-7/CD328 and TIGIT, or induced in T-cells, such as CCR7, CD2, CD3, CD4, CD8, CD28, CD45, CD96, CD229/SLAMF3, DNAM-1/CD226, CD25/IL-2 R alpha, L-Selectin/CD62L and TIGIT.

The invention comprises as a preferred aspect the introduction of an exogenous sequence encoding a CAR, or a component thereof, into HSCs, preferably under the transcriptional control of a promoter of a gene that is not expressed in HSC, more preferably a gene that is only expressed in the hematopoietic cells produced by said HSC, and even more preferably of a gene that is only expressed in T-cells or NK cells.

Combining Targeted Insertion(s) in Immune Cells of Sequences Conferring Resistance to Proteasome Inhibitors with the Inactivation of Endogenous Genomic Sequences

One particular focus of the present invention is to perform gene inactivation in primary immune cells at a locus to confer resistance to proteasome inhibitors, and in the same time introducing exogenous coding sequence(s) at said locus enhancing said resistance or conferring other therapeutic benefit, the expression of which improves the overall therapeutic potential of said engineered immune cells.

Examples of relevant exogenous coding sequences that can be inserted according to the invention have been presented above in connection with their positive effects on the therapeutic potential of the cells. Here below are presented the endogenous gene that are preferably targeted by gene targeted insertion and the advantages associated with their inactivation.

According to a preferred aspect of the invention, the insertion of the coding sequence has the effect of reducing or preventing the expression of genes involved into self and non-self recognition to reduce host versus graft disease (GVHD) reaction or immune rejection upon introduction of the allogeneic cells into a recipient patient. As previously mentioned, one of the sequence-specific reagents used in the method can for instance reduce or prevent the expression of TCR genes in primary T-cells, such as the genes encoding TCR-alpha or TCR-beta.

As another preferred aspect, one gene editing step is to reduce or prevent the expression of the β2m protein and/or another protein involved in its regulation such as C2TA (Uniprot # P33076) or in MHC recognition, such as HLA proteins. This permits the engineered immune cells to be less alloreactive when infused into patients.

By “allogeneic therapeutic use” is meant that the cells originate from a donor in view of being infused into patients having a different haplotype. Indeed, the present invention provides with an efficient method for obtaining primary cells, which can be gene edited in various gene loci involved into host-graft interaction and recognition.

Other loci may also be edited in view of improving the activity, the persistence of the therapeutic activity of the engineered primary cells as detailed here after:

### Inactivation of Checkpoint Receptors and Immune Cells Inhibitory Pathways:

According to a preferred aspect of the invention, the insertion of the exogenous coding sequence has the effect of reducing or preventing the expression of a gene involved in immune cells inhibitory pathways, in particular those referred to in the literature as encoding “immune checkpoints” (Pardoll, D. M. (2012) The blockade of immune checkpoints in cancer immunotherapy, Nature Reviews Cancer, 12:252-264).

In the sense of the present invention, “immune cells inhibitory pathways” means any gene expression in immune cells that leads to a reduction of the cytotoxic activity of the lymphocytes towards malignant or infected cells. This can be for instance a gene involved into the expression of FOXP3, which is known to drive the activity of Tregs upon T cells (moderating T-cell activity). “Immune checkpoints” are molecules in the immune system that either turn up a signal (co-stimulatory molecules) or turn down a signal of activation of an immune cell. As per the present invention, immune checkpoints more particularly designate surface proteins involved in the ligand-receptor interactions between T cells and antigen-presenting cells (APCs) that regulate the T cell response to antigen (which is mediated by peptide-major histocompatibility complex (MHC) molecule complexes that are recognized by the T cell receptor (TCR)). These interactions can occur at the initiation of T cell responses in lymph nodes (where the major APCs are dendritic cells) or in peripheral tissues or tumors (where effector responses are regulated). One important family of membrane-bound ligands that bind both co-stimulatory and inhibitory receptors is the B7 family. All of the B7 family members and their known ligands belong to the immunoglobulin superfamily. Many of the receptors for more recently identified B7 family members have not yet been identified. Tumor necrosis factor (TNF) family members that bind to cognate TNF receptor family molecules represent a second family of regulatory ligand-receptor pairs. These receptors predominantly deliver co-stimulatory signals when engaged by their cognate ligands. Another major category of signals that regulate the activation of T cells comes from soluble cytokines in the microenvironment. In other cases, activated T cells upregulate ligands, such as CD40L, that engage cognate receptors on APCs. A2aR, adenosine A2a receptor; B7RP1, B7-related protein 1; BTLA, B and T lymphocyte attenuator; GAL9, galectin 9; HVEM, herpesvirus entry mediator; ICOS, inducible T cell co-stimulator; IL, interleukin; KIR, killer cell immunoglobulin-like receptor; LAG3, lymphocyte activation gene 3; PD1, programmed cell death protein 1; PDL, PD1 ligand; TGFβ, transforming growth factor-β; TIM3, T cell membrane protein 3.

Examples of further endogenous genes, which expression could be reduced or suppressed to turn-up activation of the engineered immune cells according the present invention are listed in Table 3.

For instance, the inserted exogenous coding sequence(s) can have the effect of reducing or preventing the expression, by the engineered immune cell of at least one protein selected from PD1 (Uniprot Q15116), CTLA4 (Uniprot P16410), PPP2CA (Uniprot P67775), PPP2CB (Uniprot P62714), PTPN6 (Uniprot P29350), PTPN22 (Uniprot Q9Y2R2), LAG3 (Uniprot P18627), HAVCR2 (Uniprot Q8TDQO), BTLA (Uniprot Q7Z6A9), CD160 (Uniprot 095971), TIGIT (Uniprot Q495A1), CD96 (Uniprot P40200), CRTAM (Uniprot 095727), LAIR1 (Uniprot Q6GTX8), SIGLEC7 (Uniprot Q9Y286), SIGLEC9 (Uniprot Q9Y336), CD244 (Uniprot Q9BZW8), TNFRSF10B (Uniprot 014763), TNFRSF10A (Uniprot 000220), CASP8 (Uniprot Q14790), CASP10 (Uniprot Q92851), CASP3 (Uniprot P42574), CASP6 (Uniprot P55212), CASP7 (Uniprot P55210), FADD (Uniprot Q13158), FAS (Uniprot P25445), TGFBRII (Uniprot P37173), TGFRBRI (Uniprot Q15582), SMAD2 (Uniprot Q15796), SMAD3 (Uniprot P84022), SMAD4 (Uniprot Q13485), SMAD10 (Uniprot B7ZSB5), SKI (Uniprot P12755), SKIL (Uniprot P12757), TGIF1 (Uniprot Q15583), IL10RA (Uniprot Q13651), IL10RB (Uniprot Q08334), HMOX2 (Uniprot P30519), IL6R (Uniprot P08887), IL6ST (Uniprot P40189), EIF2AK4 (Uniprot Q9P2K8), CSK (Uniprot P41240), PAG1 (Uniprot Q9NWQ8), SIT1 (Uniprot Q9Y3P8), FOXP3 (Uniprot Q9BZS1), PRDM1 (Uniprot Q60636), BATF (Uniprot Q16520), GUCY1A2 (Uniprot P33402), GUCY1A3 (Uniprot Q02108), GUCY1B2 (Uniprot Q8BXH3) and GUCY1B3 (Uniprot Q02153). The gene editing introduced in the genes encoding the above proteins is preferably combined with an inactivation of TCR in CAR T cells.

Preference is given to inactivation of PD1 and/or CTLA4, in combination with the expression of non-endogenous immunosuppressive polypeptide, such as a PD-L1 ligand and/or CTLA-4 Ig (see also peptides of Table 1 and 2).

Without being exhaustive, Table 1 shows immune checkpoint genes that can be inactivated according to the teaching of the present invention in order to improve the efficiency and fitness of the engineered T-cells. The immune checkpoints gene are preferably selected from such genes having identity to those listed in this table involved into co-inhibitory receptor function, cell death, cytokine signaling, arginine tryptophan starvation, TCR signaling, Induced T-reg repression, transcription factors controlling exhaustion or anergy, and hypoxia mediated tolerance.

TABLE 3
Immune checkpoint genes appropriate to make allogeneic
T-cells more active for immunotherapy

| | | NCBI database gene ID |
|---|---|---|
| | Genes that can be | (Homo sapiens) |
| Pathway | inactivated in pathway | on May 13^{th}, 2014 |
| Co-inhibitory | LAG3 (CD223) | 3902 |
| receptors | HAVCR2 (TIM3) | 84868 |
| | BTLA (CD272) | 151888 |
| | CD160 (NK1) | 11126 |
| | TIGIT (VSIG9) | 201633 |
| | CD96 (TACTILE) | 10225 |
| | CRTAM (CD355) | 56253 |
| | LAIR1 (CD305) | 3903 |
| | SIGLEC7 (CD328) | 27036 |
| | A2A (IGKV2-29) | 28882 |
| | SIGLEC9 (CD329) | 27180 |
| | CD244 (2B4)) | 51744 |
| Cell death | TNFRSF10B (CD262) | 8795 |
| | TNFRSF10A (CD261) | 8797 |
| | CASP3 | 836 |
| | CASP6 | 839 |
| | CASP7 | 840 |
| | CASP8 | 841 |
| | CASP10 | 843 |
| | Arhgap5 (GFI2) | 394 |
| | Akap8i | 10270 |
| | FADD (GIG3) | 8772 |
| | FAS (RP11) | 355 |
| | Stk17b (DRAK2) | 9262 |
| Cytokine signalling | TGFBRII (AAT3) | 7048 |
| | TGFBRI | 7046 |
| | SMAD2 (JV18) | 4087 |
| | SMAD3 | 4088 |
| | SMAD4 | 4089 |
| | SMAD10 (SMAD7) | 394331 |
| | SKI (SGS) | 6497 |
| | SKIL (SNO) | 6498 |
| | TGIF1 (HPE4) | 7050 |
| | IL10RA (CD210) | 3587 |
| | IL10RB | 3588 |
| | HMOX2 (HO-2) | 3163 |
| | Jun (AP1) | 3725 |
| | Ppp3cc | 5533 |
| | Ppm1g | 5496 |
| | Socs1 | 8651 |
| | Soc3 | 9021 |
| | IL6R (CD126) | 3570 |
| | IL6ST (CD130) | 3572 |
| | Lck | 3932 |
| | Fyn | 2534 |
| | ADAP (FYB) | 2533 |
| | Carma1 (CARD11) | 84433 |
| | Bcl10 | 8915 |
| | Malt1 (IMD12) | 10892 |
| | TAK1 (NR2C2) | 7182 |
| arginine/tryptophan | EIF2AK4 (GCN2) | 440275 |
| starvation | Nuak2 | 81788 |
| TCR signalling | CSK | 1445 |
| | PAG1 (CBP) | 55824 |
| | SIT1 | 27240 |
| | CRTAM (CD355) | 56253 |
| | Egr2 (AT591) | 1959 |
| | DGK-a (DAGK) | 1606 |
| | DGK-z | 8525 |
| | Cblb | 868 |
| | Inpp5b | 3633 |
| | Ptpn2 (PTN2) | 5771 |
| | Vamp7 | 6845 |
| | Mast2 | 23139 |
| | tnk1 | 8711 |
| | stk17b (DRAK2) | 9262 |
| | Mdfic (HIC) | 29969 |
| | F11r (CD321) | 50848 |
| Induced Treg | FOXP3 (JM2) | 50943 |
| | Entpd1 (CD39) | 953 |
| Transcription | PRDM1 (blimp1) | 12142 |
| factors controlling | BATF | 10538 |
| exhaustion/anergy | Ypel2 | 388403 |
| | Ppp2r2d | 55844 |
| | Rock1 | 6093 |
| | Sbf1 | 6305 |
| | Hipk1 (MYAK) | 204851 |
| | Map3k3 | 4215 |
| | Grk6 | 2870 |
| | Eif2ak3 (PEK) | 9451 |
| | Fyn | 2534 |
| | NFAT1 (NFATC2) | 4773 |
| Hypoxia mediated | GUCY1A2 | 2977 |
| tolerance | GUCY1A3 | 2982 |
| | GUCY1B2 | 2974 |
| | GUCY1B3 | 2983 |

### Inhibiting Suppressive Cytokines/Metabolites

According to another aspect, the gene editing step conferring resistance to proteasome inhibitors as per the present invention is combined with another step that has the effect of reducing or preventing the expression of genes encoding or positively regulating suppressive cytokines or metabolites or receptors thereof, in particular TGFbeta (Uniprot:P01137), TGFbR (Uniprot:P37173), IL10 (Uniprot:P22301), IL10R (Uniprot: Q13651 and/or Q08334), A2aR (Uniprot: P29274), GCN2 (Uniprot: P15442) and PRDM1 (Uniprot: 075626).

Preference is given to engineered immune cells in which a sequence encoding IL-2, IL-12 or IL-15 replaces the sequence of at least one of the above endogenous genes.

The present invention has thus for object primary cells that are resistant to proteasome inhibitors and in which the expression of suppressive cytokines or metabolites has been reduced.

### Inducing Other Resistance to Chemotherapy Drugs

According to another aspect of the present method, the gene editing step conferring resistance to proteasome inhibitors as per the present invention is combined with another step that has the effect of reducing or preventing the expression of a gene responsible for the sensitivity of the immune cells to compounds used in standard of care treatments for cancer or infection, such as drugs purine nucleotide analogs (PNA) or 6-Mercaptopurine (6MP) and 6 thio-guanine (6TG) commonly used in chemotherapy. Reducing or inactivating the genes involved into the mode of action of such compounds (referred to as “drug sensitizing genes”) improves the resistance of the immune cells to same.

Examples of drug sensitizing gene are those encoding DCK (Uniprot P27707) with respect to the activity of PNA, such a clorofarabine and fludarabine, HPRT (Uniprot P00492) with respect to the activity of purine antimetabolites such as 6MP and 6TG, and GGH (Uniprot Q92820) with respect to the activity of antifolate drugs, in particular methotrexate.

This enables the cells of the invention resistant to proteasome inhibitors to be used after or in combination with other conventional anti-cancer chemotherapies, especially with therapies comprising clorofarabine, fludarabine, methotrexate and/or 6TG.

The present invention has thus for object primary cells that are resistant both to proteasome inhibitors and to PNA, such a clorofarabine and fludarabine, which are preferably DCK negative or DCK deficient.

The present invention has thus for object primary cells that are resistant both to proteasome inhibitors and to purine antimetabolites such as 6MP and 6TG, which are preferably HPRT negative or HPRT deficient.

The present invention has thus for object primary cells that are resistant both to proteasome inhibitors and to antifolate drugs, in particular methotrexate, which are preferably GGH negative or GGH deficient.

### Resistance to Immune-Suppressive Treatments

According to another aspect of the present invention, the gene editing step conferring resistance to proteasome inhibitors as per the present invention is combined with another step that has the effect of reducing or preventing the expression of drug targets, making said cells, for instance, also resistant to immune-depletion drug treatments. For example, such drug targets can be glucocorticoids receptors or antibody specific antigens in order to make the engineered immune cells resistant to glucocorticoids or immune depletion treatments, such as the antibody Alemtuzumab, which is used to deplete CD52 positive immune cells in many cancer treatments.

Also the method of the invention can comprise gene targeted insertion in endogenous gene(s) encoding or regulating the expression of CD52 (Uniprot P31358) and/or GR (Glucocorticoids receptor also referred to as NR3C1—Uniprot P04150).

According to a preferred embodiment the exogenous sequence encoding the CAR or one of its constituents is integrated into the gene encoding the antigen targeted by said CAR to avoid self-destruction of the immune cells.

The present invention has thus for object primary cells that are resistant both to proteasome inhibitors and to immune depleting agents, such as Alemtuzumab, and Glucocorticoids, which are preferably CD52 negative or GR negative.

Enhancing Specificity and Safety of Immune Cells

As underlined before, expressing chimeric antigen receptors (CAR) has become the state of the art to direct or improve the specificity of primary immune cells, such as T-Cells and NK-cells for treating tumors or infected cells. CARs expressed by these immune cells specifically target antigen markers at the surface of the pathological cells, which further help said immune cells to destroy these cells in-vivo. CARs are usually designed to comprise activation domains that stimulate immune cells in response to binding to a specific antigen (so-called positive CAR), but they may also comprise an inhibitory domain with the opposite effect (so-called negative CAR)(Fedorov, V. D. (2014) “Novel Approaches to Enhance the Specificity and Safety of Engineered T Cells” Cancer Journal 20 (2):160-165. Positive and negative CARs may be combined or co-expressed to finely tune the cells immune specificity depending of the various antigens present at the surface of the target cells.

The genetic sequences encoding CARs are generally introduced into the cells genome using retroviral vectors that have elevated transduction efficiency but can also be introduced at selected loci, more particularly under control of endogenous promoters by targeted gene recombination as also detailed before.

According to one aspect, while a positive CAR is introduced into the immune cell by a viral vector, a negative CAR can be introduced by targeted gene insertion and vice-versa, and be active preferably only during immune cells activation. Accordingly, the inhibitory (i.e. negative) CAR contributes to an improved specificity by preventing the immune cells to attack a given cell type that needs to be preserved. Still according to this aspect, said negative CAR can be an apoptosis CAR, meaning that said CAR comprise an apoptosis domain, such as FasL (CD95—NCBI: NP_000034.1) or a functional variant thereof, that transduces a signal inducing cell death (Eberstadt M; et al. “NMR structure and mutagenesis of the FADD (Mortl) death-effector domain” (1998) Nature. 392 (6679): 941-5).

Accordingly, the exogenous coding sequence inserted according to the invention can encode a factor that has the capability of inducing cell death, directly, in combination with, or by activating other compound(s).

As another way to enhance the safety of the primary immune cells, the exogenous coding sequence can encodes molecules that confer sensitivity of the immune cells to drugs or other exogenous substrates. Such molecules can be cytochrome(s), such as from the P450 family (Preissner S et al. (2010) “SuperCYP: a comprehensive database on Cytochrome P450 enzymes including a tool for analysis of CYP-drug interactions”. Nucleic Acids Res 38 (Database issue): D237-43), such as CYP2D6-1 (NCBI—NP_000097.3), CYP2D6-2 (NCBI—NP_001020332.2), CYP2C9 (NCBI—NP_000762.2), CYP3A4 (NCBI—NP_000762.2), CYP2C19 (NCBI—NP_000760.1) or CYP1A2 (NCBI—NP_000752.2.), thereby conferring hypersensitivity of the immune cells to a drug, such as cyclophosphamide and/or isophosphamide.

The present invention has thus for object primary cells that are resistant to proteasome inhibitors but sensitive to other drugs, such as cyclophosphamide and/or isophosphamide, which express exogenous or overexpress endogenous cytochromes.

### Suicide Gene Expression

In another aspect, since engineered T-cells can expand and persist for years after administration, it is desirable to include a safety mechanism to allow selective deletion of administrated T-cells. Thus, in some embodiments, the method of the invention can comprises the transformation of said T-cells with a recombinant suicide gene. Said recombinant suicide gene is used to reduce the risk of direct toxicity and/or uncontrolled proliferation of said T-cells once administrated in a subject. Suicide genes enable selective deletion of transformed cells in vivo. In particular, the suicide gene has the ability to convert a non-toxic pro-drug into cytotoxic drug or to express the toxic gene expression product. In other words, “Suicide gene” is a nucleic acid coding for a product, wherein the product causes cell death by itself or in the presence of other compounds.

A representative example of such a suicide gene is one which codes for thymidine kinase of herpes simplex virus. Additional examples are thymidine kinase of varicella zoster virus and the bacterial gene cytosine deaminase which can convert 5-fluorocytosine to the highly toxic compound 5-fluorouracil. Suicide genes also include as non-limiting examples caspase-9 or caspase-8 or cytosine deaminase. Caspase-9 can be activated using a specific chemical inducer of dimerization (CID). Suicide genes can also be polypeptides that are expressed at the surface of the cell and can make the cells sensitive to therapeutic monoclonal antibodies. As used herein “prodrug” means any compound useful in the methods of the present invention that can be converted to a toxic product. The prodrug is converted to a toxic product by the gene product of the suicide gene in the method of the present invention. A representative example of such a prodrug is ganciclovir which is converted in vivo to a toxic compound by HSV thymidine kinase. The ganciclovir derivative subsequently is toxic to tumor cells. Other representative examples of prodrugs include acyclovir, FIAU [1-(2-deoxy-2-fluoro-β-Darabinofuranosyl)-5-iodouracil], 6-methoxypurine arabinoside for VZV-TK, and 5-fluorocytosine for cytosine deaminase.

Other types of suicide genes can be introduced as exogenous sequences coding for external receptors that include binding domains or epitopes specifically recognized by approved therapeutic antibodies, such as rituximab or alemtuzumab. An example of such suicide gene is RQR8 described in WO2013153391. Such binding domains or epitopes can also be advantageously be inserted into the external domain and/or ScFv of a CAR as described in WO2016120216. The expression of such epitopes or binding domain at the surface of the immune cells allows their rapid depletion by injection of the therapeutic antibodies in the patient.

### Activation and Expansion of T Cells

Whether prior to or after genetic modification, the immune cells according to the present invention can be activated or expanded, even if they can activate or proliferate independently of antigen binding mechanisms. T-cells, in particular, can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo. T cells are generally expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, -2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

### Engineered Immune Cells and Populations of Immune Cells Obtainable by the Method

The present invention is also drawn to the variety of engineered immune cells obtainable according to one of the method described previously under isolated form or as part of populations of cells.

According to a preferred aspect of the invention the engineered cells are primary immune cells, such as NK cells or T-cells, which are generally part of populations of cells that may involve different types of cells. In general, such populations derive from patients or donors isolated by leukapheresis from PBMC (peripheral blood mononuclear cells).

According to a preferred aspect of the invention, more than 50% of the immune cells comprised in said population are TCR negative T-cells. According to a more preferred aspect of the invention, more than 50% of the immune cells comprised in said population are CAR positive T-cells.

The invention is more particularly drawn to populations of primary TCR negative T-cells resistant to proteasome inhibitors originating from a single donor, wherein at least 20%, preferably 30%, more preferably 50% of the cells in said population have been gene edited according to the present invention.

The present invention encompasses immune cells comprising any combinations of the different exogenous coding sequences and gene inactivation, which have been respectively and independently described above. Among these combinations are particularly preferred those combining the expression of a CAR under the transcriptional control of an endogenous promoter that is steadily active during immune cell activation and preferably independently from said activation, and the expression of an exogenous sequence encoding a cytokine, such as IL-2, IL-12 or IL-15, under the transcriptional control of a promoter that is up-regulated during the immune cell activation.

The methods described herein can result in different types of engineered immune cells, in particular T-cells, having one of the preferred genotypes or phenotypes:
[TCR]^{neg} [mutPSMB]^{pos} less alloreactive and resistant to PI;
[dcK]^{neg} [mutPSMB]^{pos} resistant to PI and resistant to purine analogues
[GR]^{neg} [mutPSMB]^{pos} resistant to PI and resistant to glucocorticoids;
[CD52]^{neg} [mutPSMB]^{pos}, resistant to PI and resistant to alemtuzumab;
[PD1]^{neg} [mutPSMB]^{pos}, resistant to PI and prone to higher activation;
[CTLA4]^{neg} [mutPSMB]^{pos} resistant to PI and prone to higher activation;
[TCR]^{neg} [β2m]^{neg} [mutPSMB]^{pos} better tolerated by recipient's immune system,
[TCR]^{neg} [HLA]^{neg} [mutPSMB]^{pos} better tolerated by recipient's immune system, HLA being preferably HLA-E or HLA-G;
[TCR]^{neg} [PD1]^{neg} [mutPSMB]^{pos} better tolerated by recipient's immune system, less alloreactive and resistant to PI;
[TCR]^{neg} [CTLA4]^{neg} [mutPSMB]^{pos} better tolerated by recipient's immune system, less alloreactive and resistant to PI;
less alloreactive and resistant to PI;
[TCR]^{neg} [dcK]^{neg} [mutPSMB]^{pos} less alloreactive, resistant to PI and resistant to purine analogues
[TCR]^{neg} [GR]^{neg} [mutPSMB]^{pos} less alloreactive, resistant to PI and resistant to glucocorticoids;
[TCR]^{neg} [CD52]^{neg} [mutPSMB]^{pos} less alloreactive, resistant to PI and resistant to alemtuzumab;
   The above cells can be also [CAR]^{pos} or [recombinant TCR]^{pos} to redirect their immune activity against specific cell markers.
   The above cells can also be modified to repress of inactivate one or several genes encoding BIM, BAK, BIK, BAX, PRKAA1, CUL3, IPO4, Rab6B, STIP1, HECTD2, BAB14306.1, COPE, DMC1, NP002070, REXO1L1P, SURF6, PRKACA, PRKACG or EZH2 as previously taught.

The invention is also drawn to a pharmaceutical composition comprising an engineered primary immune cell or immune cell population as previously described for the treatment of infection or cancer, and to a method for treating a patient in need thereof, wherein said method comprises:
preparing a population of engineered primary immune cells according to the method of the invention as previously described;
optionally, purifying or sorting said engineered primary immune cells;
activating said population of engineered primary immune cells upon or after infusion of said cells into said patient.

The invention is further drawn to a population of stably engineered proteasome inhibitor-resistant cells, endowed with a chimeric antigen receptor (CAR) for use as a treatment in a patient concomitantly treated with an efficient dose of a proteasome inhibitor.

According to one embodiment, said proteasome inhibitor is bortezomib, preferably administered sc (sub cutaneous) or iv (intraveneously). Bortezomib is generally administered at a dose ≥0.1 mg/m2, preferably ≥3 mg/m2, and more preferably ≥30 mg/m2.

According to another embodiment, said proteasome inhibitor is Carfilzomib, which is generally administered at a dose ≥2 mg/m2 and preferably ≥60 mg/m2.

According to one embodiment, said proteasome inhibitor is ixazomib, which is generally administered at a dose ≥1 mg/m2 and preferably ≥4 mg/m2 po.

### Therapeutic Compositions and Combination Therapies

The population of cells referred to above, which preferably originate from a single donor or patient, can be expanded under closed culture recipients to comply with highest manufacturing practices requirements and can be frozen prior to infusion into a patient, thereby providing “off the shelf” or “ready to use” therapeutic compositions.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermaliy, intratumorally, intranodally, intramedullary, intramuscularly, intracranially, by intravenous or intralymphatic injection, or intraperitoneally. In one embodiment, the cell compositions of the present invention are preferably administered by intravenous injection.

The administration of the cells or population of cells can consist of the administration of 103-1010 cells per kg body weight, preferably 105 to 106 cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

Accordingly, the present invention relies on methods for treating patients in need thereof, said method comprising at least one of the following steps:

(a) Providing an isolated T-cell obtainable by any one of the methods previously described;

(b) Administrating said cells to said patient.

As per the present invention, a significant number of cells originating from the same Leukapheresis can be obtained, which is critical to obtain sufficient doses for treating a patient. Although variations between populations of cells originating from various donors may be observed, the number of immune cells procured by a leukapheresis is generally about from 10⁸ to 10¹⁰ cells of PBMC. PBMC comprises several types of cells: granulocytes, monocytes and lymphocytes, among which from 30 to 60% of T-cells, which generally represents between 10⁸ to 10⁹ of primary T-cells from one donor. The method of the present invention generally ends up with a population of engineered cells that reaches generally more than about 10⁸ T-cells, more generally more than about 10⁹ T-cells, even more generally more than about 10¹⁰ T-cells, and usually more than 10¹¹ T-cells.

The invention is thus more particularly drawn to a therapeutically effective population of primary immune cells, wherein at least 30%, preferably 50%, more preferably 80% of the cells in said population have been modified according to any one the methods described herein. Said therapeutically effective population of primary immune cells, as per the present invention, can comprise immune cells that have integrated at least one exogenous genetic sequence conferring resistance to proteasome inhibitors at one of the gene loci listed in Table 6.

Such compositions or populations of cells can therefore be used as medicaments; especially for treating cancer, particularly for the treatment of lymphoma, but also for solid tumors such as melanomas, neuroblastomas, gliomas or carcinomas such as lung, breast, colon, prostate or ovary tumors in a patient in need thereof.

As mentioned previously, such medicament offers the possibility of being used in combination therapy with a proteasome inhibitor (e.g., bortezomib, carfilzomib, ixazomib, marizomib, delanzomib, oporozomib). Such combination therapy, according to the invention, can be used for treating cancer, including solid tumors and liquid tumors. Preferably, said cancer is a cancer which is typically treated with proteasome inhibitors, including, but not limited to, multiple myeloma (MM), acute myeloid leukemia (AML) and mantle cell lymphoma (MCL). This can be typically achieved by using proteasome resistant resistant KO TRAC CD19+ CAR T-cells and drug resistant KO TRAC CD123+ T-cells respectively.

Cancers that may be treated include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated with the allogeneic T-cell resistant to drugs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

The primary immune cells according to the present invention are particularly useful for treating various forms of lymphoma, in particular one of the following Lymphoma related conditions:
Adult Grade III Lymphomatoid Granulomatosis;
Anaplastic Large Cell Lymphoma;
Angioimmunoblastic T-cell Lymphoma;
Extranodal Marginal Zone B-cell Lymphoma of Mucosa-associated Lymphoid Tissue;
Intraocular Lymphoma; Nodal Marginal Zone B-cell Lymphoma;
Post-transplant Lymphoproliferative Disorder;
Primary Central Nervous System Hodgkin Lymphoma;
Primary Central Nervous System Non-Hodgkin Lymphoma;
Recurrent Adult Burkitt Lymphoma;
Recurrent Adult Diffuse Large Cell Lymphoma;
Recurrent Adult Diffuse Mixed Cell Lymphoma;
Recurrent Adult Diffuse Small Cleaved Cell Lymphoma;
Recurrent Adult Grade III Lymphomatoid Granulomatosis;
Recurrent Adult Hodgkin Lymphoma;
Recurrent Adult Immunoblastic Large Cell Lymphoma;
Recurrent Adult Lymphoblastic Lymphoma;
Recurrent Adult T-cell Leukemia/Lymphoma;
Recurrent Cutaneous T-cell Non-Hodgkin Lymphoma;
Recurrent Grade 1 Follicular Lymphoma;
Recurrent Grade 2 Follicular Lymphoma;
Recurrent Grade 3 Follicular Lymphoma;
Recurrent Mantle Cell Lymphoma;
Recurrent Marginal Zone Lymphoma;
Recurrent Mycosis Fungoides/Sezary Syndrome;
Recurrent Small Lymphocytic Lymphoma;
Splenic Marginal Zone Lymphoma;
Stage III Adult Burkitt Lymphoma;
Stage III Adult Diffuse Large Cell Lymphoma;
Stage III Adult Diffuse Mixed Cell Lymphoma;
Stage III Adult Diffuse Small Cleaved Cell Lymphoma;
Stage III Adult Hodgkin Lymphoma;
Stage III Adult Immunoblastic Large Cell Lymphoma;
Stage III Adult Lymphoblastic Lymphoma;
Stage III Adult T-cell Leukemia/Lymphoma;
Stage III Cutaneous T-cell Non-Hodgkin Lymphoma;
Stage III Grade 1 Follicular Lymphoma;
Stage III Grade 2 Follicular Lymphoma;
Stage III Grade 3 Follicular Lymphoma;
Stage III Mantle Cell Lymphoma;
Stage III Marginal Zone Lymphoma;
Stage III Mycosis Fungoides/Sezary Syndrome;
Stage III Small Lymphocytic Lymphoma;
Stage IV Adult Burkitt Lymphoma;
Stage IV Adult Diffuse Large Cell Lymphoma;
Stage IV Adult Diffuse Mixed Cell Lymphoma;
Stage IV Adult Diffuse Small Cleaved Cell Lymphoma; Stage IV Adult Hodgkin Lymphoma;
Stage IV Adult Immunoblastic Large Cell Lymphoma;
Stage IV Adult Lymphoblastic Lymphoma;
Stage IV Adult T-cell Leukemia/Lymphoma;
Stage IV Cutaneous T-cell Non-Hodgkin Lymphoma;
Stage IV Grade 1 Follicular Lymphoma;
Stage IV Grade 2 Follicular Lymphoma;
Stage IV Grade 3 Follicular Lymphoma;
Stage IV Mantle Cell Lymphoma;
Stage IV Marginal Zone Lymphoma;
Stage IV Mycosis Fungoides/Sezary Syndrome;
Stage IV Small Lymphocytic Lymphoma;
Unspecified Adult Solid Tumor, Protocol Specific;
Waldenström Macroglobulinemia

According to a preferred embodiment of the invention, the treatment according to the invention is administrated into patients undergoing an immunosuppressive treatment. The present invention preferably relies on cells or population of cells, which have been made resistant to at least one drug agent, and to a proteasome inhibitor, according to the present invention due to either expression of a drug resistance gene or the inactivation of a drug sensitizing gene. In this aspect, the drug treatment should help the selection and expansion of the T-cells according to the invention within the patient. In one embodiment, said T-cells of the invention can undergo robust in vivo expansion and can persist for an extended amount of time.

In some embodiments, the combination therapy of the present invention can further be combined with one or more others therapies against cancer selected from the group consisting of antibodies therapy, chemotherapy, cytokines therapy, dendritic cell therapy, gene therapy, hormone therapy, laser light therapy and radiation therapy.

In a further embodiment, the cell compositions of the present invention are administered to a patient in conjunction with (e.g., before, simultaneously or following) bone marrow transplantation, T-cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention.

### Pharmaceutical Composition

The isolated T-cells of the present invention may be administered either alone, or as a pharmaceutical composition in combination with diluents and/or with other components such as IL-2 or other cytokines or cell populations. Briefly, pharmaceutical compositions of the present invention may comprise T-cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In a preferred embodiment CAR-expressing engineered immune cells are administered in combination with a proteasome inhibitor and at least one of the following treatments: an alkylating agent, preferably Bendamustine or Melphalan, an anti-inflammatory agent, preferably a corticosteroid, more preferably Dexamethasone, or Prednisone, a therapeutic antibody, preferably an anti-CD20 and more preferably Rituximab, an antineoplasic agent, preferably Docetaxel, an Histone deacetylase inhibitor (HDAC inhibitor), preferably Romidepsin and more preferably Romidepsin and Belinostat, a chelating agent, preferably Samarium (153Sm) Lexidronam pentasodium, an inhibitor of heat shock protein 90, preferably Tanespimycin, a pyrimidine analog, preferably Cytarabine and more preferably Cytarabine Cytarabine+Daunorubine, an intercalating agent.

In specific embodiments the following treatments are given to patients in combination with CAR-expressing engineered immune cells and a proteasome inhibitor, said patients being treated for a corresponding condition as in Table 4.

TABLE 4
Treatment(s) combined to CAR-expressing engineered
immune cells and a proteasome inhibitor

| Associated treatment | Condition |
|---|---|
| Bendamustine | relapsed/refractory multiple myeloma |
| Bendamustine + Rituximab | relapsed/refractory indolent and mantle cell |
| | non-Hodgkin lymphoma |
| Dexamethasone | previously untreated multiple myeloma |
| Docetaxel | previously treated advanced NSCL |
| Romidepsin + Belinostat | chronic lymphocytic leukemia |
| Rimidepsin Samarium | relapsed/refractory multiple myeloma |
| Lexidronam | |
| Tanespimycin | relapsed/refractory multiple myeloma |
| Vorinostat | glioblastoma |
| Cytarabine + Daunorubine | acute myeloid leukemia |
| Melphalan + Prednisone | initial treatment of multiple myeloma |

According to one embodiment, the dose of proteasome inhibitor at which the object of the present invention is resistant to corresponds but is not limited to, in the case of bortezomib, to a dose of more than 0.05 mg/m²/dose IV or SC or more than 0.1 mg/m²/dose IV or SC or at least 0.13 mg/m²/dose IV or SC, preferably of more than 1.3 mg/m²/dose IV or SC or more preferably SC. In general, the previous doses are administered twice weekly for 2 weeks (days 1, 4, 8, 11). In some cases, it is followed by a 10-day rest period (days 12 to 21) before starting again for six to 8 3-week cycles.

In a particular embodiment, bortezomib can be given in combination with rituximab (375 mg/m² IV), cyclophosphamide (750 mg/m² IV), and doxorubicin (50 mg/m² IV) on day 1, plus prednisone 100 mg/m² IV on days 1-5. This is especially used in the case of multiple myeloma.

Carfilzomib, which irreversibly binds to and inhibits the chymotrypsin-like activity of the 20S proteasome is more particularly used in combination therapy with the proteasome inhibitor resistant engineered immune cells of the present invention for relapsed and refractory multiple myeloma.

According to one embodiment of the invention, the dose of carfilzomib with which the proteasome inhibitor resistant engineered immune cells of the present invention can be used is preferably more than 5 mg/m² IV, preferably more than 20 mg/m² IV, more preferably more than 50 mg/m² IV, the higher doses being infused over 30 minutes.

In the case of ixazomib, a dose given with the proteasome inhibitor resistant engineered immune cells of the present invention is more than 0.25 and preferably less than 2.5 mg/m2 per oral administration (po). In another embodiment a dose of ixazomib given with the proteasome inhibitor resistant engineered immune cells of the present invention is more than 0.1 to more than 0.4 mg/m2, preferably the dose of ixazomib is more than 1.5 mg/m2 to more than 4 mg/m2. In general, these doses are given orally.

In a specific embodiment, a dose of ixazomib given with the proteasome inhibitor resistant engineered immune cells of the present invention is more than 0.4 mg po, more preferably more than 4 mg PO, generally on days 1, 8, and 15 of a 28-day cycle.

According to another embodiment, the combination of the present invention can be administered to a patient in the need thereof in the presence of Lenalidomide and Dexamethasone.

According to a specific embodiment, the present invention relates to a method of treatment using for instance VELCADE® (bortezomib) in combination with anti-CD123 CART T-cells (TCR KO) in the Treatment of AML. VELCADE® is approved by the Food and Drug Administration (FDA) for the treatment of multiple myeloma in patients who have received at least two prior therapies and have demonstrated disease progression on their last therapy. Its effectiveness is also being tested in other cancers. Under such treatment, the primary outcome measures can be performed:
Response to anti-CD123 CART T-cells (TCR KO) in combination with Bortezomib (VELCADE®)
Response is the primary endpoint of this study and will be scored on day 21 (3 weeks after the first dose of anti-CD123 CART T-cells (TCR KO) and VELCADE) and every 3 weeks subsequently. Patients are evaluated for response in an organ if they have AGVHD in that organ at the start of treatment with VELCADE or if AGVHD develops after the start of VELCADE, but before the time period of evaluation. Complete response in an organ is defined as no evidence clinical or biochemical signs of AGVHD. For the overall assessment, it is defined as complete resolution of rash, abnormal LFTs, and absence of diarrhea attributed to AGVHD.
Partial response is defined as a one stage decrease in any organ system without worsening in other organ systems.
Number of Toxicities Related to Bortezomib (VELCADE®), especially any toxicities of VELCADE® when administered to recipients of allogeneic hematopoietic stem cell transplant in the setting of steroid refractory or steroid dependent acute graft-versus-host disease.
   Inclusion Criteria for this treatment can be:
Patients must have undergone an allogeneic HSCT
Clinical or histological evidence of AGVHD
Has been treated with a minimum of 2 mg/kg of methylprednisolone per day or equivalent dose of steroids and either one of the following:
   Has had a minimum of 3 days of steroids including the day of assignment and has progressive disease.
   Has had a minimum of 7 days of steroids including the day of assignment and has had no response.
   AGVHD progresses at anytime when steroids are tapered to less than 2 mg/kg/day of methylprednisolone or its equivalent.

### Other Definitions

The terms “therapeutic agent”, “chemotherapeutic agent”, or “drug” as used herein refers to a compound or a derivative thereof that can interact with a cancer cell, thereby reducing the proliferative status of the cell and/or killing the cell. Examples of chemotherapeutic agents include, but are not limited to, proteasome inhibitors as previously referred in the present specification, alkylating agents (e.g., cyclophosphamide, ifosamide), metabolic antagonists (e.g., purine nucleoside antimetabolite such as clofarabine, fludarabine or 2′-deoxyadenosine, 25 methotrexate (MTX), 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, and the like. Such agents may further include, but are not limited to, the anti-cancera gents TRIMETHOTRIXATE™ (TMTX), TEMOZOLOMIDE™, RALTRITREXED™, S-(4-Nitrobenzyl)-6-thioinosine (NBMPR), 6-benzyguanidine (6-BG), bis-chloronitrosourea (BCNU) and CAMPTOTHECIN™, 30 or a therapeutic derivative of any thereof.
Amino acid residues in a polypeptide sequence are designated herein according to the one-letter code, in which, for example, Q means Gin or Glutamine residue, R means Arg or Arginine residue and D means Asp or Aspartic acid residue.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“nucleic acid” or “nucleic acid molecule” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Nucleic acids can be either single stranded or double stranded.
By “gene” is meant the basic unit of heredity, consisting of a segment of DNA arranged in a linear manner along a chromosome, which codes for a specific protein or segment of protein, small RNA and the like. A gene typically includes at least a promoter, a 5′ untranslated region, one or more coding sequences (exons), optionally introns, a 3′ untranslated region. The gene may further comprise a terminator, enhancers and/or silencers.
By “genome” it is meant the entire genetic material contained in a cell such as nuclear genome, chloroplastic genome, mitochondrial genome, preferably nuclear genome.
By “mutation” is intended the substitution, deletion, insertion of one or more nucleotides/amino acids, preferably in a polynucleotide (cDNA, gene) or a polypeptide sequence. Said mutation can affect the coding sequence of a gene or its regulatory sequence. It may also affect the structure of the genomic sequence or the structure/stability of the encoded mRNA. A stable mutation is a mutation reverting with a frequency of less than 10⁻⁷. Similarly a stably engineered cell is a cell which keeps the acquired phenotype for at least 5 cell cycles, preferably more than 10 cell cycles, and even more preferably more than 10⁴ generations.
“rare-cutting endonuclease” refers to a wild type or variant enzyme capable of catalyzing the hydrolysis (cleavage) of bonds between nucleic acids within a DNA or RNA molecule, preferably a DNA molecule. Particularly, said nuclease can be an endonuclease, more preferably a rare-cutting endonuclease which is highly specific, recognizing nucleic acid target sites ranging from 10 to 45 base pairs (bp) in length, usually ranging from 10 to 35 base pairs in length. The endonuclease according to the present invention recognizes and cleaves nucleic acid at specific polynucleotide sequences, further referred to as “target sequence”. The rare-cutting endonuclease can recognize and generate a single- or double-strand break at specific polynucleotides sequences. In a particular embodiment, said rare-cutting endonuclease according to the present invention can be a CRISPR/Cas9 endonuclease from the type II prokaryotic CRISPR (Clustered Regularly Interspaced Short Palindromic Repeats) adaptive immune system (see for review (Sorek, R., et al. (2013). “CRISPR-mediated Adaptive Immune Systems in Bacteria and Archaea.” Annu Rev Biochem)). The CRISPR Associated (Cas) system was first discovered in bacteria and functions as a defense against foreign DNA, either viral or plasmid. CRISPR-mediated genome engineering first proceeds by the selection of target sequence often flanked by a short sequence motif, referred as the proto-spacer adjacent motif (PAM). Following target sequence selection, a specific crRNA, complementary to this target sequence is engineered. Trans-activating crRNA (tracrRNA) required in the CRISPR type II systems paired to the crRNA and bound to the provided Cas9 protein. Cas9 acts as a molecular anchor facilitating the base pairing of tracRNA with cRNA. In this ternary complex, the dual tracrRNA:crRNA structure acts as guide RNA that directs the endonuclease Cas9 to the cognate target sequence. Target recognition by the Cas9-tracrRNA:crRNA complex is initiated by scanning the target sequence for homology between the target sequence and the crRNA. In addition to the target sequence-crRNA complementarity, DNA targeting requires the presence of a short motif adjacent to the protospacer (protospacer adjacent motif—PAM). Following pairing between the dual-RNA and the target sequence, Cas9 subsequently introduces a blunt double strand break 3 bases upstream of the PAM motif. In the present invention, guide RNA can be designed for example to specifically target a gene encoding a TCR component. Following the pairing between the guide RNA and the target sequence, Cas9 induces a cleavage within the TCR gene. Rare-cutting endonuclease can also be a homing endonuclease, also known under the name of meganuclease. Such homing endonucleases are well-known to the art (Stoddard, B. L. (2005) “Homing endonuclease structure and function.” Q Rev Biophys 38(1): 49-95). Other rare-cutting endonucleases are “TALE-nuclease”, which respectively refer to engineered proteins resulting from the fusion of a DNA binding domain typically derived from Transcription Activator like Effector proteins (TALE) with an endonuclease catalytic domain. Such catalytic domain is preferably a nuclease domain and more preferably a domain having endonuclease activity, like for instance I-Tevl, and Fok-I. In a particular embodiment, said nuclease is a monomeric TALE-Nuclease (see WO2012138927). TALE-nuclease have been widely described and used to stimulate gene targeting and gene modifications (Christian, M., T. Cermak, et al. (2010). “Targeting DNA double-strand breaks with TAL effector nucleases.” Genetics 186(2): 757-61.). Such engineered TALE-nucleases are commercially available under the trade name TALEN® (Cellectis, 8 rue de la Croix Jarry, 75013 Paris, France).
The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.
By “chimeric antigen receptor” (CAR) it is meant a chimeric receptor which comprises an extracellular ligand-binding domain, a transmembrane domain and a signaling transducing domain.
The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. In a preferred embodiment, said extracellular ligand-binding domain comprises a single chain antibody fragment (scFv) comprising the light (VL) and the heavy (VH) variable fragment of a target antigen specific monoclonal antibody joined by a flexible linker. In a preferred embodiment, said scFV is derived from a CD19, CD22 or a CD123 antibody.
The terms “vector” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.
By “delivery vector” is intended any delivery vector which can be used in the present invention to put into cell contact (i.e. “contacting”) or deliver inside cells or subcellular compartments (i.e. “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors.
Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Preferably, viral vectors are lentiviral vectors or AAV vectors.
By “lentiviral vector” is meant vectors derived from lentiviruses, preferably HIV-Based lentiviral vectors having a large packaging capacity, reduced immunogenicity and ability to stably transduce with high efficiency a large range of different cell types. Lentiviral vectors are generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as non-limiting example, that are able to integrate the genome of a target cell. At the opposite by “non-integrative lentiviral vectors (or NILV)” is meant efficient gene delivery vectors that do not integrate the genome of a target cell through the action of the virus integrase. In a preferred embodiment lentiviral vectors are used cells of the present invention transduced using the same, more preferably integrative lentiviral vectors.
By “cell” or “cells” is intended any living cells, preferably any eukaryotic cell, a mammalian cell and more preferably a primary human living cells and a cell or a cell line derived from these primary human living cells.
Because some variability may arise from the genomic data from which these polypeptides derive, and also to take into account the possibility to substitute some of the amino acids present in these polypeptides without significant loss of activity (functional variants), the term “sharing identity with” reflects this variability. The invention therefore encompasses polypeptide or polynucleotide variants that may share at least 70%, preferably at least 80%, more preferably at least 90% and even more preferably at least 95% identity with the sequences provided in this patent application. The present invention is thus drawn to polypeptides comprising a polypeptide sequence that has at least 70%, preferably at least 80%, more preferably at least 90%, 95% 97% or 99% sequence identity with the amino acid sequence referred to in the present specification.
“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.
«Knock-out» or “KO” means that the gene is mutated or deleted to that extend it cannot express a functional product.
“TRAC” refers to “T-cell receptor alpha and/or beta constant» and corresponds to TCRα and or b subunit constant gene.

In addition to the preceding features, the invention comprises further features which will emerge from the following examples illustrating the method of engineering allogeneic and resistant T-cells for immunotherapy, as well as to the appended drawings.

### EXAMPLES

### Example 1: T-Cells and CAR-T Cells Depletion by Increasing Doses of Bortezomib

### Experimental Protocol

Peripheral blood mononuclear cells (PBMC) were obtained from healthy volunteer donors as described by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284).

To investigate whether bortezomib treatment modify the survival of primary cells, frozen PBMCs were thawed and activated using Dynabeads human T activator CD3/CD28. 3 days after their activation, 1 million cells were transduced using a CD123 CAR (specific for CD123) at a MOI of 5. Cells were then immediately diluted in X-Vivo-15 media supplemented by 20 ng/ml IL-2 and 5% human serum AB and diluted at 1×10⁶ cells/ml and kept in culture at 37° C. in the presence of 5% CO₂. 7 days later, T cells, CART cells and MOLM13 cells were cultured for 48h in the presence of bortezomib increasing doses of bortezomib ranging from 0 nM to 100 pM. At the end of the culture cell viability was assessed by flow cytometry using a live dead cell marker.

The results displayed in FIG. 1 show that primary immune cells, expressing a CAR (CART cells) and MOLM13 cells are equally depleted by bortezomib treatment with an EC50 of about 10 nM.

### Example 2: Co-Transduction of a CAR Along with an ORF Rendering Cells Resistant to Bortezomib

3 ORFs encoding 3 different proteins conferring resistance to Bortezomib have been cloned into lentiviral vectors along with a sequence encoding GFP separated by a T2A to detect protein expression using flow cytometry analysis as previously described.

TABLE 5
Exogenous coding sequences used in the examples

| SEQ | | |
|---|---|---|
| ID NO. | Description | Sequence |
| 1 | POMP-T2A-GFP | GGCGCGCCAGTCCTCCGACAGACTGAGTC |
| | | GCCCGGGGGCCACCATGAACGCAAGGGGG |
| | | CTGGGGTCCGAACTGAAAGATAGTATTCC |
| | | CGTCACCGAACTGTCCGCATCAGGGCCAT |
| | | TTGAGAGCCACGATCTGCTGAGAAAGGGC |
| | | TTTAGCTGCGTGAAGAACGAGCTGCTGCC |
| | | ATCCCACCCCCTGGAGCTGTCTGAGAAGA |
| | | ACTTCCAGCTGAATCAGGACAAGATGAAC |
| | | TTTTCCACCCTGAGGAATATCCAGGGCCT |
| | | GTTCGCCCCCCTGAAGCTCCAGATGGAGT |
| | | TTAAGGCAGTGCAGCAGGTGCAGCGGCTG |
| | | CCCTTCCTGAGCAGCAGCAACCTGTCTCT |
| | | GGACGTGCTGAGGGGCAATGACGAGACAA |
| | | TCGGCTTCGAGGACATCCTGAACGATCCC |
| | | AGCCAGTCCGAAGTGATGGGCGAGCCTCA |
| | | CCTGATGGTGGAGTACAAGCTGGGCCTGC |
| | | TGGGCAGCGGCGAGGGCAGAGGCTCCCTG |
| | | CTGACATGCGGCGATGTGGAGGAGAATCC |
| | | CGGCCCTATGTCCGGCGGAGAGGAGCTGT |
| | | TCGCAGGAATCGTGCCCGTGCTGATCGAG |
| | | CTGGACGGCGATGTGCACGGCCACAAGTT |
| | | TTCTGTGCGCGGAGAGGGAGAGGGCGACG |
| | | CCGATTATGGCAAGCTGGAGATCAAGTTC |
| | | ATCTGTACCACAGGCAAGCTGCCAGTGCC |
| | | CTGGCCTACCCTGGTGACCACACTGTGCT |
| | | ACGGCATCCAGTGTTTTGCCCGGTATCCA |
| | | GAGCACATGAAGATGAACGACTTCTTTAA |
| | | GAGCGCCATGCCCGAGGGCTACATCCAGG |
| | | AGAGGACAATCCAGTTCCAGGACGATGGC |
| | | AAGTATAAGACCCGCGGCGAGGTGAAGTT |
| | | TGAGGGCGATACACTGGTGAACCGGATCG |
| | | AGCTGAAGGGCAAGGACTTCAAGGAGGAT |
| | | GGCAATATCCTGGGCCACAAGCTGGAGTA |
| | | CTCTTTTAACAGCCACAACGTGTACATCC |
| | | GCCCCGACAAGGCCAACAATGGCCTGGAG |
| | | GCCAACTTCAAGACCAGGCACAATATCGA |
| | | GGGAGGAGGAGTGCAGCTGGCAGACCACT |
| | | ACCAGACAAACGTGCCTCTGGGCGATGGC |
| | | CCTGTGCTGATCCCAATCAATCACTATCT |
| | | GTCTACCCAGACAAAGATCAGCAAGGACC |
| | | GGAATGAGGCCAGAGATCACATGGTGCTG |
| | | CTGGAATCTTTCTCCGCTTGTTGTCACAC |
| | | TCACGGGATGGACGAACTGTATCGCTAAC |
| | | CTGCAGGGGCGCGCCAGTCCTCCGACAGA |
| | | CTGAGTCGCCCG |
| 2 | PSMB5-T2A-GFP | GGGGCCACCATGGCACTGGCATCCGTGCT |
| | | GGAGAGGCCCCTGCCTGTGAACCAGAGGG |
| | | GCTTCTTTGGCCTGGGCGGCAGAGCCGAC |
| | | CTGCTGGATCTGGGCCCAGGCTCTCTGAG |
| | | CGACGGCCTGTCTCTGGCAGCCCCTGGCT |
| | | GGGGAGTGCCTGAGGAGCCAGGCATCGAG |
| | | ATGCTGCACGGCACCACAACCCTGGCCTT |
| | | CAAGTTTCGGCACGGCGTGATCGTGGCCG |
| | | CCGACTCTAGAGCCACAGCCGGCGCCTAT |
| | | ATCGCCAGCCAGACCGTGAAGAAAGTGAT |
| | | CGAGATCAACCCTTACCTGCTGGGAACAA |
| | | TGGCCGGAGGAGCCGCCGATTGCAGCTTT |
| | | TGGGAGAGGCTGCTGGCCAGGCAGTGTCG |
| | | CATCTATGAGCTGCGGAACAAGGAGAGAA |
| | | TCAGCGTGGCTGCCGCCTCCAAGCTGCTG |
| | | GCCAATATGGTGTACCAGTATAAGGGCAT |
| | | GGGCCTGAGCATGGGCACAATGATCTGCG |
| | | GATGGGACAAGAGGGGCCCCGGCCTGTAC |
| | | TATGTGGATTCTGAGGGCAATCGCATCTC |
| | | TGGCGCCACCTTCAGCGTGGGCAGCGGCA |
| | | GCGTGTACGCCTACGGCGTGATGGACAGA |
| | | GGCTACAGCTATGATCTGGAGGTGGAGCA |
| | | GGCTTACGACCTGGCCCGGCGGGCCATCT |
| | | ACCAGGCCACCTATAGGGATGCCTACTCC |
| | | GGCGGAGCAGTGAACCTGTATCACGTGCG |
| | | GGAGGACGGCTGGATCAGAGTGAGCAGCG |
| | | ACAATGTGGCCGATCTGCACGAGAAGTAC |
| | | AGCGGCTCCACACCAGGCTCCGGCGAGGG |
| | | CCGCGGCTCTCTGCTGACCTGCGGCGATG |
| | | TGGAGGAGAACCCAGGCCCCATGTCTGGC |
| | | GGAGAGGAGCTGTTCGCAGGAATCGTGCC |
| | | CGTGCTGATCGAGCTGGACGGCGATGTGC |
| | | ACGGCCACAAGTTTAGCGTGCGCGGAGAG |
| | | GGAGAGGGCGACGCCGATTACGGCAAGCT |
| | | GGAGATCAAGTTCATCTGTACAACCGGCA |
| | | AGCTGCCTGTGCCCTGGCCCACACTGGTG |
| | | ACAACCCTGTGCTATGGCATCCAGTGTTT |
| | | TGCCCGGTACCCTGAGCACATGAAGATGA |
| | | ATGACTTCTTTAAGTCCGCCATGCCAGAG |
| | | GGCTATATCCAGGAGCGGACCATCCAGTT |
| | | CCAGGACGATGGCAAGTACAAGACAAGAG |
| | | GCGAGGTGAAGTTTGAGGGCGATACCCTG |
| | | GTGAACAGGATCGAGCTGAAGGGCAAGGA |
| | | CTTCAAGGAGGATGGCAATATCCTGGGCC |
| | | ACAAGCTGGAGTATTCCTTTAACTCTCAC |
| | | AACGTGTACATCCGCCCCGACAAGGCCAA |
| | | CAATGGCCTGGAGGCCAACTTTAAGACAC |
| | | GGCACAATATCGAGGGAGGAGGAGTGCAG |
| | | CTGGCAGACCACTATCAGACCAACGTGCC |
| | | TCTGGGCGATGGCCCCGTGCTGATCCCTA |
| | | TCAATCACTACCTGAGCACACAGACCAAG |
| | | ATCAGCAAGGACAGGAATGAGGCCCGCGA |
| | | TCACATGGTGCTGCTGGAGTCTTTCAGCG |
| | | CCTGCTGTCACACCCACGGCATGGATGAG |
| | | CTGTACAGATGACCTGCAGG |
| 3 | mutated | GGCGCGCCAGTCCTCCGACAGACTGAGTC |
| | PSMB5-T2A-GFP | GCCCGGGGGCCACCATGGCACTGGCATCC |
| | | GTGCTGGAGAGGCCCCTGCCTGTGAACCA |
| | | GAGGGGCTTCTTTGGCCTGGGCGGCAGAG |
| | | CCGACCTGCTGGATCTGGGCCCAGGCTCT |
| | | CTGAGCGACGGCCTGTCTCTGGCAGCCCC |
| | | TGGCTGGGGAGTGCCTGAGGAGCCAGGCA |
| | | TCGAGATGCTGCACGGCACCACAACCCTG |
| | | GCCTTCAAGTTTCGGCACGGCGTGATCGT |
| | | GGCCGCCGACTCTAGAGCCACAGCCGGCG |
| | | CCTATATCGCCAGCCAGACCGTGAAGAAA |
| | | GTGATCGAGATCAACCCTTACCTGCTGGG |
| | | AACAATGGCCGGAGGAACAGTGGATTGCA |
| | | GCTTTTGGGAGAGGCTGCTGGCCAGGCAG |
| | | TGTCGCATCTATGAGCTGCGGAACAAGGA |
| | | GAGAATCAGCGTGGCTGCCGCCTCCAAGC |
| | | TGCTGGCCAATATGGTGTACCAGTATAAG |
| | | GGCATGGGCCTGAGCATGGGCACAATGAT |
| | | CTGCGGATGGGACAAGAGGGGCCCCGGCC |
| | | TGTACTATGTGGATTCTGAGGGCAATCGC |
| | | ATCTCTGGCGCCACCTTCAGCGTGGGCAG |
| | | CGGCAGCGTGTACGCCTACGGCGTGATGG |
| | | ACAGAGGCTACAGCTATGATCTGGAGGTG |
| | | GAGCAGGCTTACGACCTGGCCCGGCGGGC |
| | | CATCTACCAGGCCACCTATAGGGATGCCT |
| | | ACTCCGGCGGAGCAGTGAACCTGTATCAC |
| | | GTGCGGGAGGACGGCTGGATCAGAGTGAG |
| | | CAGCGACAATGTGGCCGATCTGCACGAGA |
| | | AGTACAGCGGCTCCACACCAGGCTCCGGC |
| | | GAGGGCCGCGGCTCTCTGCTGACCTGCGG |
| | | CGATGTGGAGGAGAACCCAGGCCCCATGT |
| | | CTGGCGGAGAGGAGCTGTTCGCAGGAATC |
| | | GTGCCCGTGCTGATCGAGCTGGACGGCGA |
| | | TGTGCACGGCCACAAGTTTAGCGTGCGCG |
| | | GAGAGGGAGAGGGCGACGCCGATTACGGC |
| | | AAGCTGGAGATCAAGTTCATCTGTACAAC |
| | | CGGCAAGCTGCCTGTGCCCTGGCCCACAC |
| | | TGGTGACAACCCTGTGCTATGGCATCCAG |
| | | TGTTTTGCCCGGTACCCTGAGCACATGAA |
| | | GATGAATGACTTCTTTAAGTCCGCCATGC |
| | | CAGAGGGCTATATCCAGGAGCGGACCATC |
| | | CAGTTCCAGGACGATGGCAAGTACAAGAC |
| | | AAGAGGCGAGGTGAAGTTTGAGGGCGATA |
| | | CCCTGGTGAACAGGATCGAGCTGAAGGGC |
| | | AAGGACTTCAAGGAGGATGGCAATATCCT |
| | | GGGCCACAAGCTGGAGTATTCCTTTAACT |
| | | CTCACAACGTGTACATCCGCCCCGACAAG |
| | | GCCAACAATGGCCTGGAGGCCAACTTTAA |
| | | GACACGGCACAATATCGAGGGAGGAGGAG |
| | | TGCAGCTGGCAGACCACTATCAGACCAAC |
| | | GTGCCTCTGGGCGATGGCCCCGTGCTGAT |
| | | CCCTATCAATCACTACCTGAGCACACAGA |
| | | CCAAGATCAGCAAGGACAGGAATGAGGCC |
| | | CGCGATCACATGGTGCTGCTGGAGTCTTT |
| | | CAGCGCCTGCTGTCACACCCACGGCATGG |
| | | ATGAGCTGTACAGATGACCTGCAGG |
| 4 | PSMB5-Uniprot | MALASVLERPLPVNQRGFFGLGGRADLLD |
| | # P28074 | LGPGSLSDGLSLAAPGWGVPEEPGIEMLH |
| | | GTTTLAFKFRHGVIVAADSRATAGAYIAS |
| | | QTVKKVIEINPYLLGTMAGGAADCSFWER |
| | | LLARQCRIYELRNKERISVAAASKLLANM |
| | | VYQYKGMGLSMGTMICGWDKRGPGLYYVD |
| | | SEGNRISGATFSVGSGSVYAYGVMDRGYS |
| | | YDLEVEQAYDLARRAIYQATYRDAYSGGA |
| | | VNLYHVREDGWIRVSSDNVADLHEKYSGS |
| | | TP |

### Experimental Protocol

To investigate whether primary cells endowed with a CAR could be transduced with a protein responsible for resistance to bortezomib treatment, frozen PBMCs were thawed and activated using Dynabeads human T activator CD3/CD28. 3 days after their activation 1 million T-cells were transduced using the CD123 CAR and one of the ORF encoding a gene according to the present invention at a MOI of 5 for both lentiviral particles produced in-house. Cells were then immediately diluted in X-Vivo-15 media supplemented by 20 ng/ml IL-2 and 5% human serum AB and diluted at 1×10⁶ cells/ml and kept in culture at 37° C. in the presence of 5% CO2. 4 and 7 days later, CAR expression was detected by basis fetoprotein (BFP) expression and POMP, PSMB5 or mutated PSMB5 expression was detected by GFP expression were assessed by flow cytometry. At the end of the culture cell viability was assessed by flow cytometry using a live dead cell marker.

### Results

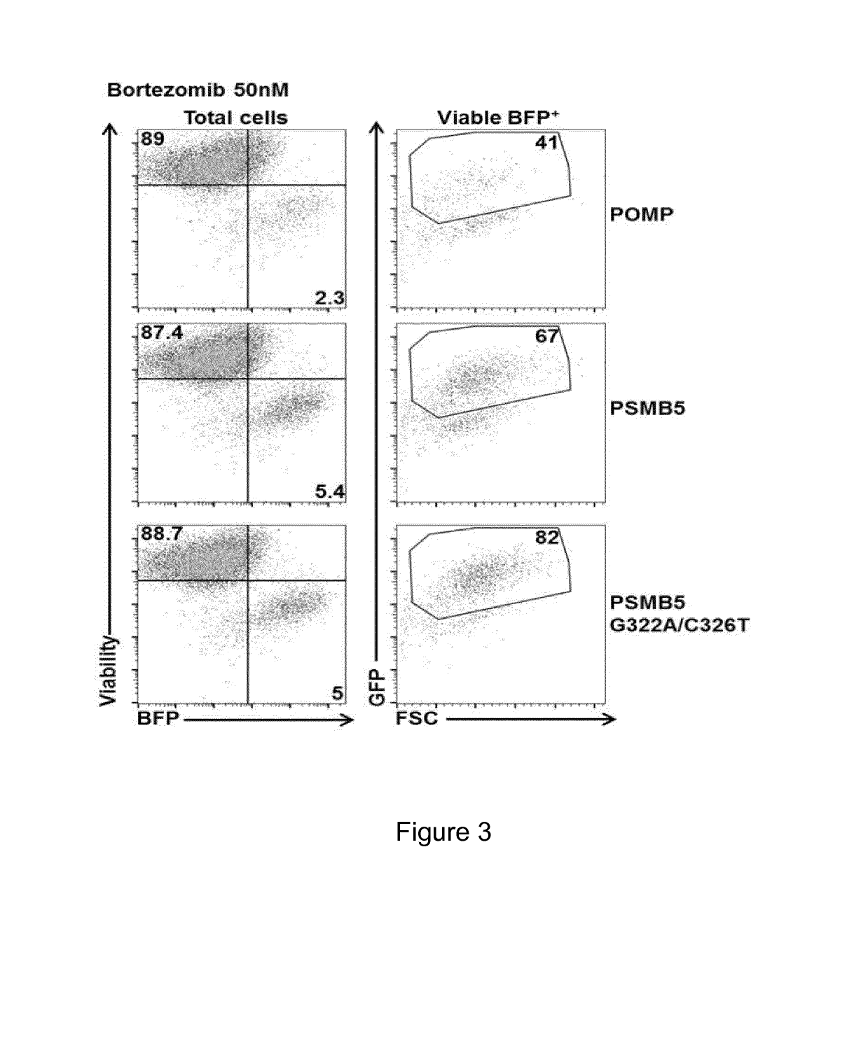

The results, shown in FIGS. 2 and 3, demonstrated for the first time that primary immune cells endowed with a CAR and expressing a gene conferring resistance to a PI can be prepared.

The frequency of CART cells that coexpress either POMP, PSMB5 or mutated PSMB5 ranges from 32.6 to 43.2% at day 4, and ranges from 41.3 to 60.5% at d7. The results showed that the number of cells that co-express CAR CD123 and a mutated PSMB5 is higher than the frequency of CAR T-cells that co-express POMP or PSMB5. This indicated that expressing a mutated PSMB5 of the invention confers an advantage (survival proliferation) to CAR-T cells.

### Example 3: CART Cells Coexpress PSMB5 or Mutated PSMB5 Better Resist to Bortezomib Treatment than CART Cells and CART Cells that Coexpress POMP

### Experimental Protocol

To investigate whether overexpressing POMP, PSMB5 or mutated PSMB5 on CART cells confers resistance to bortezomib treatment, T-cells frozen PBMCs were thawed and activated using Dynabeads human T activator CD3/CD28. 3 days after their activation 1 million T-cells were transduced or not using the CD123 CAR and one of the ORF at a MOI of 5 for both lentiviral particles. Cells were then immediately diluted in X-Vivo-15 media supplemented by 20 ng/ml IL-2 and 5% human serum AB and diluted at 1×10⁶ cells/ml and kept in culture at 37° C. in the presence of 5% CO₂. 7 days later, T cells, CART cells and MOLM13 cells were cultured for 48h in the presence of increasing doses of bortezomib ranging from 0 to 100 μM. At the end of the culture cell viability was assessed by flow cytometry using a live dead cell marker.

### Results

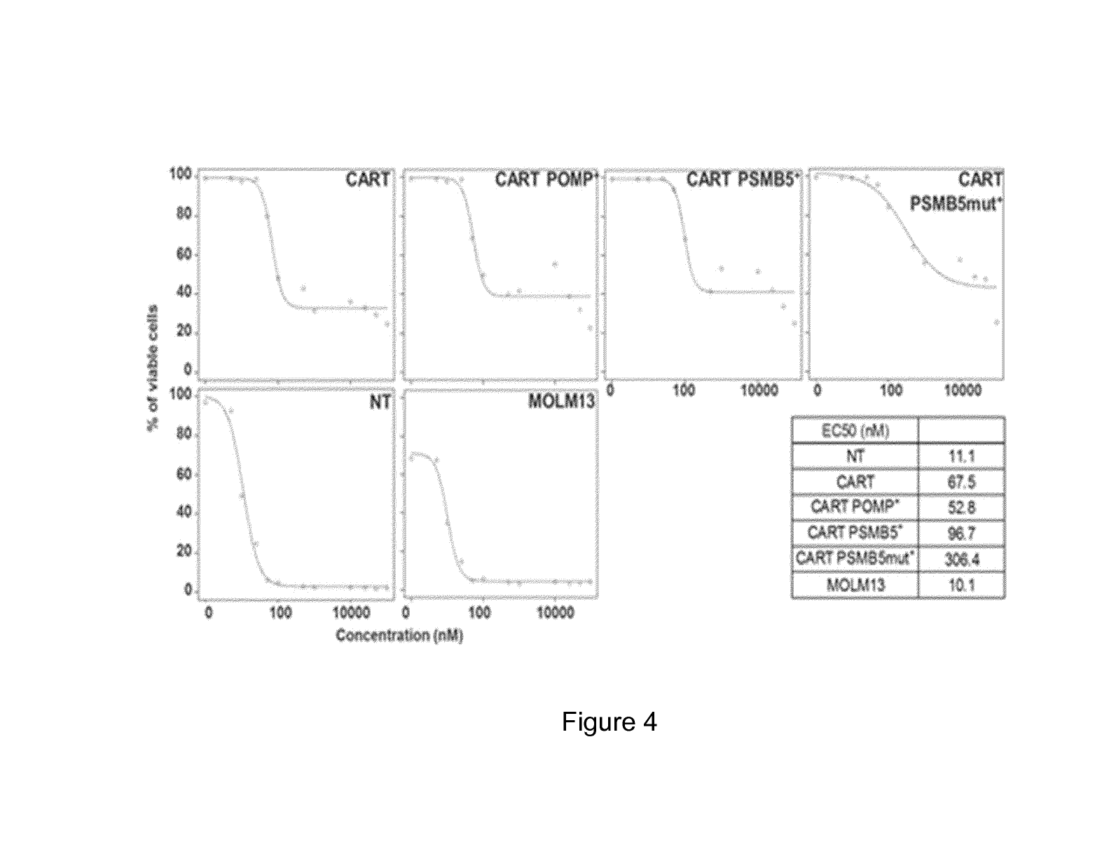

The results displayed in FIG. 4 show that CART cells that coexpress PSMB5 or mutated PSMB5 are enriched when CART cells are cultured in the presence of 50 nM of bortezomib. CART cells that express mutated PSMB5 are even more resistant to bortezomib treatment than CART cells that express PSMB5. The EC50 of bortezomib is increased for CART cells as compared to non-transduced cells and even more for CART cells that express PSMB5, and even more for CART cells that express mutated PSMB5, as compared to WT CART cells and NT cells.

### Example 4: Identification of Immune Primary Cells Loci Involved into Cells Sensitivity to Bortezomib by Using Improved Genome-Scale CRISPR Knock-Out Library

A genome-scale library of DNA sequences encoding a large diversity of guide-RNAs, referred to as GECKO has been cloned into lentiviral particles prepared according to the protocol described in Shalem et al. (Genome-Scale CRISPR-Cas9 Knockout Screening in Human Cells (2014) Science, 343:84-87).

Peripheral blood mononuclear cells (PBMC) were obtained from healthy volunteer donors and T-lymphocytes were purified using the EasySep human T-cell enrichment kit (Stemcell Technologies). By contrast with the GECKO protocol described by Shalem et al., primary T-cells were activated and immediately transduced with the viral particles. The T-cells were first mixed with dynabeads (25 uL/10⁶ T-cells) in Xvivo-15 media and then plated on 12 well plates coated by 30 ug/mL retronectin per well (iml of retronectin per well incubated 1 hour at 37° C. followed by a washing steps using 1 ml of PBS 2% FBS) in a total volume of 400 μL. The mixture was supplemented by either 200 μL of GECKO lentiviral particles or 200 μL of Xvivo-15 media and incubated 2H at 37° C. and 5% CO2. The mixture was then supplemented by 600 μL of Xvivo-15 media, 10% AB serum and 40 ng/mL IL2 and incubated overnight at 37° C. with 5% CO2. Cells were then washed with 1.5 ml of Xvivo-15 media and resuspended in Xvivo-15 media, 5% AB serum, 20 ng/mL IL-2 in 6 wells plate at 10⁶ cells/mL.

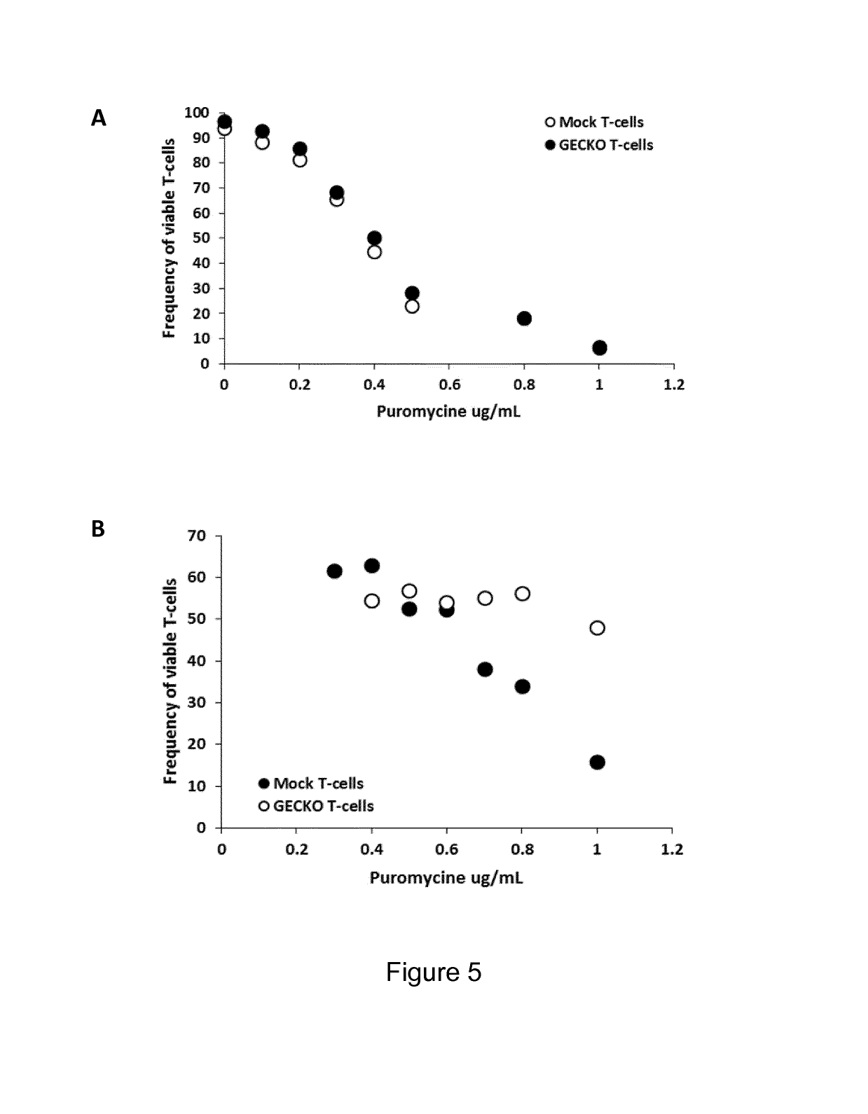

3 days post transduction, cells were recovered and used to perform a puromycine resistance test. This test consisted in incubating mock or transduced T-cells (10⁶ cells/mL) in 96 well plate (100 μL/well) in the presence of increasing concentration of puromycine (0-1 pg/mL) for 3 days at 37° C. and in the presence of 5% CO2. FIG. 2 illustrates the puromycine sensitivity pattern of mock- and GECKO-transduced primary T-cells determined by flow cytometry. Our data showed that GECKO-transduced primary T-cells were more resistance than mock-T-cells as indicated by the difference of viability frequency obtained between both cellular entities incubated in the presence of 1 pg/ml puromycine (see FIG. 5). Conventional puromycine resistance test is described in the following. 3 days post transduction (i.e. 6 days after activation), T-cells are recovered and used to perform a puromycine resistance test. This test consisted in incubating mock or transduced T-cells (10⁶ cells/mL) in 96 well plate (100 μL/well) in the presence of increasing concentration of puromycine (0-1 pg/mL) for 3 days at 37° C. and in the presence of 5% CO2. Cells are then recovered, labeled using EFluor780 dye and analyzed by flow cytometry to determine their viability. Their viability is plotted as a function of puromycine concentration to determine puromycine IC50.

The data show that transduction of the primary T-cells, when performed on the same day as their activation, significantly improves the efficiency of the GECKO library.

TABLE 6
Preferred human endogenous gene loci responsive to T-cell activation

| | | | | T.8Eff.Sp.OT1. | T.8Eff.Sp.OT1. | T.8Eff.Sp.OT1. |
|---|---|---|---|---|---|---|
| symbol | description | inductionRatio12hr | T.8Nve.Sp.OT1 | 12hr.LisOva | 48hr.LisOva | d6.LisOva |
| Il3 | interleukin 21 | 16.4 | 12.8 | 208.9 | 18.4 | 13.6 |
| Il2 | interleukin 3 | 97.0 | 16.0 | 1554.4 | 17.7 | 18.1 |
| Ccl4 | isopentenyl-diphosphate delta isomerase 2 | 2.1 | 16.8 | 35.6 | 17.6 | 19.7 |
| Il21 | granzyme C | 9.2 | 17.4 | 160.5 | 20.4 | 24.9 |
| Gp49a | chemokine (C-C motif) receptor 8 | 5.9 | 18.5 | 108.4 | 31.5 | 20.9 |
| Cxcl10 | interleukin 2 | 58.4 | 21.1 | 1229.6 | 32.7 | 17.9 |
| Nr4a3 | interleukin 1 receptor, type 1 | 2.6 | 21.2 | 54.6 | 35.5 | 21.7 |
| Lilrb4 | tumor necrosis factor (ligand) superfamily, | 4.1 | 21.8 | 88.8 | 29.3 | 20.0 |
| | member 4 | | | | | |
| Cd200 | neuronal calcium sensor 1 | 4.5 | 24.1 | 109.6 | 46.3 | 23.2 |
| Cdkn1a | CDK5 and Abl enzyme substrate 1 | 3.1 | 26.2 | 80.9 | 49.1 | 32.8 |
| Gzmc | transmembrane and tetratricopeptide | 2.0 | 26.8 | 53.9 | 26.2 | 29.4 |
| | repeat containing 2 | | | | | |
| Nr4a2 | LON peptidase N-terminal domain and | 3.2 | 28.4 | 90.4 | 50.4 | 28.3 |
| | ring finger 1 | | | | | |
| Cish | glycoprotein 49 A | 15.0 | 31.6 | 472.4 | 30.6 | 212.5 |
| Nr4a1 | polo-like kinase 2 | 3.6 | 31.7 | 114.3 | 39.0 | 32.5 |
| Tnf | lipase, endothelial | 2.1 | 32.4 | 66.7 | 35.9 | 33.3 |
| Ccr8 | cyclin-dependent kinase inhibitor 1A | 9.7 | 34.6 | 335.4 | 54.4 | 71.0 |
| | (P21) | | | | | |
| Lad1 | grainyhead-like 1 (Drosophila) | 2.1 | 35.1 | 73.4 | 52.0 | 44.1 |
| Slamf1 | cellular retinoic acid binding protein II | 5.3 | 35.4 | 187.2 | 43.3 | 36.3 |
| Crabp2 | adenylate kinase 4 | 2.2 | 35.9 | 80.4 | 58.5 | 39.8 |
| Furin | microtubule-associated protein 1B | 2.1 | 36.2 | 77.7 | 36.4 | 38.4 |
| Gadd45g | acyl-CoA synthetase long-chain family | 2.0 | 37.2 | 76.0 | 45.2 | 41.3 |
| | member 6 | | | | | |
| Bcl2l1 | zinc finger E-box binding homeobox 2 | 2.1 | 38.6 | 80.7 | 44.9 | 455.4 |
| Ncs1 | CD200 antigen | 9.8 | 41.2 | 404.3 | 70.4 | 36.8 |
| Ciart | carboxypeptidase D | 3.1 | 41.6 | 127.7 | 71.4 | 71.6 |
| Ahr | thioredoxin reductase 3 | 3.6 | 43.4 | 157.8 | 61.7 | 28.8 |
| Spry1 | myosin IE | 2.3 | 43.6 | 100.2 | 61.3 | 77.0 |
| Tnfsf4 | RNA binding protein with multiple | 2.1 | 43.6 | 91.5 | 49.8 | 36.5 |
| | splicing 2 | | | | | |
| Myo10 | mitogen-activated protein kinase kinase 3, | 2.9 | 44.8 | 127.9 | 66.4 | 43.1 |
| | opposite strand | | | | | |
| Dusp5 | PERP, TP53 apoptosis effector | 2.8 | 44.9 | 127.2 | 78.4 | 72.4 |
| Myc | myosin X | 4.1 | 45.5 | 184.9 | 81.6 | 57.5 |
| Psrc1 | immediate early response 3 | 2.7 | 45.6 | 121.6 | 63.9 | 66.2 |
| St6galnac4 | folliculin interacting protein 2 | 2.6 | 47.5 | 124.2 | 87.4 | 96.6 |
| Nfkbid | leukocyte immunoglobulin-like receptor, | 9.9 | 48.9 | 483.3 | 64.5 | 179.1 |
| | subfamily B, member 4 | | | | | |
| Bst2 | circadian associated repressor of | 4.5 | 50.6 | 225.5 | 100.3 | 33.8 |
| | transcription | | | | | |
| Txnrd3 | RAR-related orphan receptor gamma | 2.1 | 51.7 | 106.7 | 47.5 | 52.8 |
| Plk2 | proline/serine-rich coiled-coil 1 | 3.9 | 52.9 | 205.9 | 92.3 | 79.6 |
| Gfi1 | cysteine rich protein 2 | 2.4 | 54.2 | 127.7 | 90.3 | 182.9 |
| Pim1 | cAMP responsive element modulator | 2.0 | 55.7 | 112.6 | 54.4 | 57.3 |
| Pvt1 | chemokine (C-C motif) ligand 4 | 20.2 | 55.8 | 1125.8 | 103.1 | 89.0 |
| Nfkbib | nuclear receptor subfamily 4, group A, | 7.8 | 58.5 | 457.6 | 78.7 | 72.0 |
| | member 2 | | | | | |
| Gnl2 | transglutaminase 2, C polypeptide | 2.3 | 58.7 | 132.1 | 69.8 | 64.7 |
| Cd69 | synapse defective 1, Rho GTPase, | 2.1 | 62.5 | 132.7 | 111.3 | 31.0 |
| | homolog 2 (C. elegans) | | | | | |
| Dgat2 | sprouty homolog 1 (Drosophila) | 4.2 | 63.8 | 268.5 | 76.8 | 61.4 |
| Atf3 | activating transcription factor 3 | 3.2 | 65.8 | 210.3 | 88.3 | 75.8 |
| Tnfrsf21 | pogo transposable element with KRAB | 2.9 | 68.6 | 196.9 | 91.1 | 293.2 |
| | domain | | | | | |
| Lonrf1 | tumor necrosis factor receptor | 3.2 | 70.6 | 224.5 | 126.5 | 72.9 |
| | superfamily, member 21 | | | | | |
| Cables1 | cytokine inducible SH2-containing protein | 7.5 | 74.3 | 558.7 | 82.5 | 133.9 |
| Cpd | lymphotoxin A | 2.6 | 74.6 | 197.2 | 93.4 | 58.6 |
| Qtrtd1 | FBJ osteosarcoma oncogene | 3.0 | 74.9 | 224.1 | 89.0 | 61.1 |
| Polr3d | signaling lymphocytic activation molecule | 5.4 | 75.6 | 412.0 | 108.4 | 190.4 |
| | family member 1 | | | | | |
| Kcnq5 | syndecan 3 | 2.4 | 76.0 | 180.0 | 77.2 | 85.3 |
| Fos | mitochondrial ribosomal protein L47 | 2.1 | 77.2 | 161.7 | 152.0 | 72.3 |
| Slc19a2 | ladinin | 5.5 | 77.3 | 423.2 | 152.5 | 70.4 |
| Hif1a | E2F transcription factor 5 | 2.5 | 77.7 | 198.0 | 92.0 | 65.2 |
| Il15ra | ISG15 ubiquitin-like modifier | 2.8 | 77.9 | 221.0 | 88.9 | 45.1 |
| Nfkb1 | aryl-hydrocarbon receptor | 4.2 | 78.7 | 333.2 | 145.7 | 91.4 |
| Phlda3 | diacylglycerol O-acyltransferase 2 | 3.2 | 81.0 | 259.2 | 150.0 | 84.4 |
| Mtrr | FBJ osteosarcoma oncogene B | 2.0 | 81.3 | 163.7 | 139.3 | 98.5 |
| Pogk | pleckstrin homology-like domain, | 2.9 | 84.8 | 244.5 | 126.9 | 83.8 |
| | family A, member 3 | | | | | |
| Map2k3os | potassium voltage-gated channel, | 3.0 | 86.3 | 261.0 | 118.1 | 63.4 |
| | subfamily Q, member 5 | | | | | |
| Egr2 | tumor necrosis factor receptor | 2.5 | 88.6 | 219.0 | 106.1 | 51.0 |
| | superfamily, member 10b | | | | | |
| Isg15 | Mir17 host gene 1 (non-protein coding) | 2.1 | 90.4 | 190.1 | 120.0 | 51.2 |
| Perp | glucose-fructose oxidoreductase domain | 2.2 | 92.9 | 208.5 | 168.7 | 237.4 |
| | containing 1 | | | | | |
| Ipo4 | plexin A1 | 2.1 | 94.8 | 200.7 | 118.0 | 90.3 |
| Mphosph10 | heat shock factor 2 | 2.4 | 96.8 | 233.2 | 191.0 | 104.8 |
| Plk3 | carbohydrate sulfotransferase 11 | 2.4 | 96.8 | 235.1 | 180.8 | 385.7 |
| Ifitm3 | growth arrest and DNA-damage-inducible | 4.8 | 104.6 | 504.8 | 109.3 | 95.0 |
| | 45 gamma | | | | | |
| Polr1b | solute carrier family 5 (sodium-dependent | 2.1 | 107.0 | 227.3 | 192.8 | 75.8 |
| | vitamin transporter), member 6 | | | | | |
| Usp18 | interferon induced transmembrane | 2.8 | 109.2 | 302.6 | 43.9 | 106.4 |
| | protein 3 | | | | | |
| Top1mt | DENN/MADD domain containing 5A | 2.6 | 109.5 | 279.9 | 102.0 | 517.4 |
| Dkc1 | plasminogen activator, urokinase receptor | 2.1 | 112.4 | 234.8 | 55.7 | 57.3 |
| Polr1c | solute carrier family 19 (thiamine | 3.0 | 115.4 | 343.1 | 221.7 | 138.4 |
| | transporter), member 2 | | | | | |
| Cdk6 | ubiquitin domain containing 2 | 2.2 | 117.4 | 255.7 | 198.9 | 122.2 |
| Ier3 | nuclear receptor subfamily 4, group A, | 11.8 | 118.0 | 1394.1 | 114.2 | 69.6 |
| | member 3 | | | | | |
| Lta | zinc finger protein 52 | 2.5 | 118.8 | 295.6 | 160.9 | 167.4 |
| Ptprs | SH3 domain containing ring finger 1 | 2.4 | 119.3 | 280.9 | 116.5 | 156.5 |
| Fnip2 | dihydrouridine synthase 2 | 2.1 | 122.7 | 260.3 | 237.7 | 202.8 |
| Asna1 | cyclin-dependent kinase 5, regulatory | 2.1 | 122.7 | 259.3 | 168.4 | 124.0 |
| | subunit 1 (p35) | | | | | |
| Mybbp1a | processing of precursor 7, ribonuclease P | 2.1 | 125.9 | 264.9 | 235.7 | 150.6 |
| | family, (S, cerevisiae) | | | | | |
| Il1r1 | growth factor independent 1 | 3.5 | 126.8 | 437.7 | 212.0 | 156.6 |
| Dennd5a | interleukin 15 receptor, alpha chain | 2.9 | 130.9 | 380.1 | 144.3 | 167.8 |
| E2f5 | BCL2-like 1 | 4.7 | 133.7 | 627.4 | 257.4 | 231.2 |
| Rcl1 | protein tyrosine phosphatase, receptor | 2.6 | 136.6 | 358.8 | 157.5 | 125.0 |
| | type, S | | | | | |
| Fosl2 | plasmacytoma variant translocation 1 | 3.4 | 136.7 | 465.5 | 179.8 | 140.7 |
| Atad3a | fos-like antigen 2 | 2.5 | 137.0 | 347.5 | 107.2 | 177.8 |
| Bax | BCL2-associated X protein | 2.5 | 138.0 | 347.3 | 260.1 | 150.2 |
| Phf6 | solute carrier family 4, sodium bicarbonate | 2.3 | 140.3 | 328.2 | 258.7 | 397.5 |
| | cotransporter, member 7 | | | | | |
| Zfp52 | tumor necrosis factor receptor | 2.2 | 141.7 | 311.1 | 161.7 | 111.6 |
| | superfamily, member 4 | | | | | |
| Crtam | chemokine (C—X—C motif) ligand 10 | 12.7 | 141.7 | 1798.3 | 242.1 | 59.4 |
| Nop14 | polo-like kinase 3 | 2.8 | 144.8 | 406.3 | 200.1 | 119.9 |
| Rel | CD3E antigen, epsilon polypeptide | 2.2 | 158.7 | 350.2 | 260.9 | 111.4 |
| | associated protein | | | | | |
| Gramd1b | tumor necrosis factor (ligand) superfamily, | 2.1 | 162.4 | 342.1 | 242.1 | 169.7 |
| | member 11 | | | | | |
| Ifi27l2a | polymerase (RNA) III (DNA directed) | 3.0 | 166.3 | 503.7 | 296.1 | 121.6 |
| | polypeptide D | | | | | |
| Tnfrsf10b | early growth response 2 | 2.8 | 173.5 | 494.0 | 136.3 | 68.2 |
| Rpl7l1 | DnaJ (Hsp40) homolog, subfamily C, | 2.1 | 173.6 | 369.4 | 346.2 | 254.3 |
| | member 2 | | | | | |
| Eif1a | DNA topoisomerase 1, mitochondrial | 2.7 | 182.2 | 498.2 | 338.6 | 114.4 |
| Nfkb2 | tripartite motif-containing 30D | 2.3 | 182.6 | 423.4 | 65.8 | 90.6 |
| Heatr1 | DnaJ (Hsp40) homolog, subfamily C, | 2.0 | 190.1 | 389.4 | 285.5 | 228.2 |
| | member 21 | | | | | |
| Utp20 | SAM domain, SH3 domain and nuclear | 2.2 | 191.5 | 422.1 | 222.8 | 304.1 |
| | localization signals, 1 | | | | | |
| Chst11 | solute carrier family 5 (inositol | 2.1 | 191.6 | 400.2 | 210.0 | 123.4 |
| | transporters), member 3 | | | | | |
| Ddx21 | mitochondrial ribosomal protein L15 | 2.1 | 191.6 | 396.3 | 329.8 | 137.7 |
| Hsf2 | dual specificity phosphatase 5 | 4.0 | 203.5 | 818.1 | 307.5 | 560.7 |
| Bccip | apoptosis enhancing nuclease | 2.3 | 211.1 | 478.5 | 288.2 | 137.9 |
| Tagap | ets variant 6 | 2.3 | 218.3 | 508.1 | 220.5 | 297.3 |
| Sdc3 | DIM1 dimethyladenosine transferase | 2.2 | 218.4 | 486.0 | 356.0 | 129.7 |
| | 1-like (S, cerevisiae) | | | | | |
| Sytl3 | 2′-5′ oligoadenylate synthetase-like 1 | 2.1 | 229.0 | 473.3 | 130.7 | 124.3 |
| Gtpbp4 | UTP18, small subunit (SSU) processome | 2.1 | 232.0 | 494.3 | 384.9 | 189.5 |
| | component, homolog (yeast) | | | | | |
| Crip2 | BRCA2 and CDKN1A interacting protein | 2.4 | 234.6 | 563.3 | 437.5 | 269.8 |
| Sh3rf1 | synaptotagmin-like 3 | 2.4 | 242.4 | 572.9 | 316.7 | 700.7 |
| Nsfl1c | 5-methyltetrahydrofolate-homocysteine | 2.9 | 245.7 | 706.5 | 334.6 | 150.6 |
| | methyltransferase reductase | | | | | |
| Gtf2f1 | URB2 ribosome biogenesis 2 homolog | 2.0 | 245.7 | 500.2 | 489.8 | 184.6 |
| | (S, cerevisiae) | | | | | |
| Slc4a7 | ubiquitin-conjugating enzyme E2C | 2.1 | 251.2 | 530.5 | 288.2 | 85.2 |
| | binding protein | | | | | |
| Etv6 | lysine (K)-specific demethylase 2B | 2.2 | 251.8 | 547.1 | 332.7 | 262.1 |
| Trim30d | queuine tRNA-ribosyltransferase domain | 3.0 | 260.3 | 788.7 | 358.0 | 75.5 |
| | containing 1 | | | | | |
| Ddx27 | ubiquitin specific peptidase 31 | 2.0 | 265.2 | 533.2 | 277.1 | 176.2 |
| Pwp2 | eukaryotic translation initiation factor 2- | 2.0 | 267.7 | 540.5 | 260.8 | 244.8 |
| | alpha kinase 2 | | | | | |
| Chchd2 | ATPase family, AAA domain containing | 2.5 | 268.8 | 679.7 | 523.1 | 147.1 |
| | 3A | | | | | |
| Myo1e | adhesion molecule, interacts with CXADR | 2.3 | 269.5 | 610.9 | 272.9 | 182.8 |
| | antigen 1 | | | | | |
| Eif5b | SUMO/sentrin specific peptidase 3 | 2.0 | 272.5 | 548.7 | 544.5 | 298.4 |
| Stat5a | ESF1, nucleolar pre-rRNA processing | 2.2 | 276.3 | 610.4 | 482.2 | 266.5 |
| | protein, homolog (S, cerevisiae) | | | | | |
| Cops6 | deoxynucleotidyltransferase, terminal, | 2.1 | 282.9 | 600.4 | 359.9 | 326.1 |
| | interacting protein 2 | | | | | |
| D19Bwg1357e | TGFB-induced factor homeobox 1 | 2.1 | 300.5 | 618.9 | 217.5 | 210.6 |
| Aatf | eukaryotic translation initiation factor 1A | 2.5 | 300.8 | 738.7 | 597.7 | 262.8 |
| Aen | interferon-stimulated protein | 2.1 | 305.7 | 651.2 | 144.3 | 138.4 |
| Amica1 | pleiomorphic adenoma gene-like 2 | 2.1 | 311.5 | 651.9 | 376.2 | 405.9 |
| Wdr43 | PWP2 periodic tryptophan protein | 2.3 | 321.8 | 743.3 | 586.5 | 189.3 |
| | homolog (yeast) | | | | | |
| Cct4 | furin (paired basic amino acid cleaving | 5.2 | 329.7 | 1728.3 | 271.7 | 421.5 |
| | enzyme) | | | | | |
| Nifk | tumor necrosis factor | 6.6 | 330.7 | 2188.4 | 489.9 | 213.3 |
| Tgm2 | apoptosis antagonizing transcription factor | 2.3 | 331.4 | 754.8 | 523.1 | 221.5 |
| Ero1l | interferon, alpha-inducible protein 27 like | 2.5 | 334.0 | 828.1 | 296.0 | 221.4 |
| | 2A | | | | | |
| Gfod1 | ST6 (alpha-N-acetyl-neuraminyl-2,3-beta- | 3.9 | 338.4 | 1311.3 | 636.0 | 298.2 |
| | galactosyl-1,3)-N-acetylgalactosaminide | | | | | |
| | alpha-2,6-sialyltransferase 4 | | | | | |
| Ak4 | methyltransferase like 1 | 2.2 | 339.4 | 744.7 | 662.8 | 94.5 |
| Sdad1 | notchless homolog 1 (Drosophila) | 2.0 | 339.4 | 690.3 | 610.3 | 158.1 |
| Dimt1 | mitochondrial ribosomal protein L3 | 2.1 | 340.0 | 725.5 | 651.4 | 359.8 |
| Esf1 | UBX domain protein 2A | 2.1 | 343.8 | 732.9 | 532.1 | 428.5 |
| Cd3eap | guanine nucleotide binding protein-like 2 | 3.2 | 347.6 | 1124.7 | 647.4 | 227.5 |
| | (nucleolar) | | | | | |
| Samsn1 | programmed cell death 11 | 2.0 | 353.9 | 711.8 | 435.9 | 287.4 |
| Tnfrsf4 | cyclin-dependent kinase 8 | 2.0 | 364.0 | 731.1 | 702.5 | 346.2 |
| Mettl1 | eukaryotic translation initiation factor 5B | 2.3 | 365.1 | 838.2 | 544.5 | 355.5 |
| Cd274 | RNA terminal phosphate cyclase-like 1 | 2.5 | 373.3 | 948.8 | 746.4 | 155.8 |
| Ubtd2 | NSFL1 (p97) cofactor (p47) | 2.3 | 374.1 | 876.1 | 725.9 | 369.7 |
| Icos | nuclear factor of kappa light polypeptide | 3.9 | 378.5 | 1465.1 | 389.9 | 224.0 |
| | gene enhancer in B cells inhibitor, delta | | | | | |
| Kdm2b | M-phase phosphoprotein 10 (U3 small | 2.8 | 379.8 | 1069.3 | 738.4 | 290.8 |
| | nucleolar ribonucleoprotein) | | | | | |
| Larp4 | GRAM domain containing 1B | 2.5 | 382.7 | 949.6 | 363.4 | 659.2 |
| Eif3d | ERO1-like (S, cerevisiae) | 2.2 | 387.7 | 872.3 | 773.0 | 520.9 |
| Tnfaip3 | nuclear receptor subfamily 4, group A, | 6.8 | 387.8 | 2639.0 | 343.7 | 220.7 |
| | member 1 | | | | | |
| Map1b | surfeit gene 2 | 2.1 | 399.8 | 852.2 | 696.3 | 204.0 |
| Cdv3 | N(alpha)-acetyltransferase 25, NatB | 2.1 | 405.7 | 847.3 | 669.5 | 194.1 |
| | auxiliary subunit | | | | | |
| Plac8 | yrdC domain containing (E, coli) | 2.0 | 406.7 | 830.8 | 635.3 | 267.0 |
| Mrpl3 | La ribonucleoprotein domain family, | 2.2 | 408.8 | 887.9 | 586.6 | 358.3 |
| | member 4 | | | | | |
| Surf2 | SDA1 domain containing 1 | 2.2 | 419.8 | 939.9 | 631.4 | 284.7 |
| Ubxn2a | importin 4 | 2.8 | 420.3 | 1183.6 | 777.8 | 173.5 |
| Utp18 | inducible T cell co-stimulator | 2.2 | 423.9 | 920.9 | 818.8 | 796.9 |
| Isg20 | solute carrier family 7 (cationic amino | 2.1 | 439.4 | 934.4 | 842.6 | 344.6 |
| | acid transporter, y+ system), member 1 | | | | | |
| Dnajc2 | arsA arsenite transporter, ATP-binding, | 2.6 | 446.6 | 1165.0 | 717.9 | 963.9 |
| | homolog 1 (bacterial) | | | | | |
| Jak2 | polymerase (RNA) I polypeptide C | 2.7 | 447.8 | 1208.4 | 854.0 | 295.9 |
| Slc7a1 | spermatogenesis associated 5 | 2.0 | 450.8 | 920.2 | 516.0 | 361.6 |
| Syde2 | ubiquitin specific peptidase 18 | 2.7 | 451.8 | 1240.5 | 296.0 | 250.7 |
| Slc5a6 | placenta-specific 8 | 2.1 | 452.4 | 967.3 | 888.6 | 590.8 |
| Dnttip2 | general transcription factor IIF, | 2.3 | 454.8 | 1063.9 | 890.0 | 680.8 |
| | polypeptide 1 | | | | | |
| Idi2 | nuclear factor of kappa light polypeptide | 3.4 | 456.4 | 1535.5 | 679.1 | 502.7 |
| | gene enhancer in B cells inhibitor, beta | | | | | |
| Dus2 | PHD finger protein 6 | 2.5 | 462.0 | 1159.5 | 775.8 | 510.4 |
| Pitrm1 | RRN3 RNA polymerase I transcription | 2.1 | 462.2 | 948.4 | 913.2 | 388.9 |
| | factor homolog (yeast) | | | | | |
| Plxna1 | cytotoxic and regulatory T cell molecule | 2.5 | 473.7 | 1177.8 | 586.8 | 431.8 |
| Cdk5r1 | COP9 (constitutive photomorphogenic) | 2.3 | 483.6 | 1101.9 | 947.8 | 560.3 |
| | homolog, subunit 6 (Arabidopsis thaliana) | | | | | |
| Ube2cbp | asparagine-linked glycosylation 3 | 2.1 | 485.9 | 1006.3 | 758.7 | 339.4 |
| | (alpha-1,3-mannosyltransferase) | | | | | |
| Tnfsf11 | tryptophanyl-tRNA synthetase | 2.0 | 486.1 | 987.1 | 897.1 | 504.7 |
| Pop7 | hypoxia up-regulated 1 | 2.0 | 494.3 | 996.6 | 802.4 | 690.3 |
| Psme3 | family with sequence similarity 60, | 2.0 | 500.8 | 1002.1 | 834.7 | 417.6 |
| | member A | | | | | |
| Mir17hg | bone marrow stromal cell antigen 2 | 3.8 | 502.5 | 1922.9 | 925.5 | 246.0 |
| Tsr1 | nuclear factor of kappa light polypeptide | 2.4 | 503.2 | 1231.8 | 494.0 | 341.8 |
| | gene enhancer in B cells 2, p49/p100 | | | | | |
| Rbpms2 | UTP20, small subunit (SSU) processome | 2.4 | 510.5 | 1240.2 | 696.4 | 245.8 |
| | component, homolog (yeast) | | | | | |
| Mrpl47 | CD274 antigen | 2.2 | 516.6 | 1128.7 | 246.9 | 220.2 |
| Rab8b | proviral integration site 1 | 3.4 | 518.4 | 1766.4 | 676.9 | 970.0 |
| Plagl2 | signal transducer and activator of | 2.3 | 530.0 | 1210.4 | 496.6 | 507.8 |
| | transcription 5A | | | | | |
| Grhl1 | CD69 antigen | 3.2 | 535.7 | 1725.8 | 289.5 | 153.9 |
| Zeb2 | pitrilysin metallepetidase 1 | 2.1 | 544.9 | 1153.8 | 968.4 | 349.3 |
| sept-02 | cyclin-dependent kinase 6 | 2.7 | 550.3 | 1476.5 | 1064.0 | 642.1 |
| Slc5a3 | DEAD (Asp-Glu-Ala-Asp) box | 2.3 | 556.2 | 1286.9 | 987.2 | 480.4 |
| | polypeptide 27 | | | | | |
| Naa25 | polymerase (RNA) I polypeptide B | 2.8 | 556.2 | 1536.0 | 1070.4 | 201.3 |
| Plaur | tumor necrosis factor, alpha-induced | 2.2 | 560.6 | 1212.2 | 255.5 | 446.0 |
| | protein 3 | | | | | |
| Metap1 | nodal modulator 1 | 2.1 | 563.0 | 1161.0 | 988.9 | 439.8 |
| Alg3 | NOP14 nucleolar protein | 2.5 | 570.9 | 1418.9 | 925.3 | 398.0 |
| Mrpl15 | ribosomal protein L7-like 1 | 2.5 | 586.7 | 1448.7 | 1030.2 | 687.2 |
| Oasl1 | methionyl aminopeptidase 1 | 2.1 | 597.5 | 1244.1 | 1139.3 | 433.4 |
| Rorc | hypoxia inducible factor 1, alpha subunit | 3.0 | 624.2 | 1854.6 | 809.4 | 838.4 |
| Nomo1 | Janus kinase 2 | 2.1 | 624.5 | 1328.7 | 390.6 | 917.8 |
| Tgif1 | nuclear factor of kappa light polypeptide | 2.9 | 661.5 | 1913.3 | 713.9 | 720.5 |
| | gene enhancer in B cells 1, p105 | | | | | |
| Lipg | reticuloendotheliosis oncogene | 2.5 | 678.9 | 1686.4 | 409.8 | 580.5 |
| Rrn3 | septin 2 | 2.1 | 687.3 | 1436.0 | 1354.1 | 1181.3 |
| Dnajc21 | nucleolar protein interacting with the FHA | 2.3 | 733.4 | 1658.2 | 1280.0 | 407.2 |
| | domain of MKI67 | | | | | |
| Yrdc | elongation factor Tu GTP binding domain | 2.0 | 739.3 | 1483.5 | 1439.0 | 904.3 |
| | containing 2 | | | | | |
| Acsl6 | myelocytomatosis oncogene | 4.0 | 761.0 | 3022.8 | 1064.0 | 211.5 |
| Spata5 | dyskeratosis congenita 1, dyskerin | 2.7 | 778.2 | 2112.0 | 1549.5 | 484.2 |
| Urb2 | carnitine deficiency-associated gene | 2.1 | 801.6 | 1718.2 | 1274.7 | 1010.3 |
| | expressed in ventricle 3 | | | | | |
| Nle1 | GTP binding protein 4 | 2.4 | 824.2 | 1942.6 | 1578.7 | 567.3 |
| Wars | HEAT repeat containing 1 | 2.4 | 830.3 | 2020.6 | 1235.5 | 495.4 |
| Crem | proteaseome (prosome, macropain) | 2.1 | 838.4 | 1763.5 | 1471.1 | 936.1 |
| | activator subunit 3 (PA28 gamma, Ki) | | | | | |
| Larp1 | La ribonucleoprotein domain family, | 2.0 | 861.7 | 1742.1 | 1250.9 | 854.3 |
| member 1 | | | | | | |
| Eif2ak2 | DNA segment, Chr 19, Brigham & | 2.3 | 868.6 | 1978.4 | 1218.0 | 653.4 |
| | Women's Genetics 1357 expressed | | | | | |
| Hyou1 | eukaryotic translation initiation factor 3, | 2.2 | 909.1 | 1971.6 | 1641.9 | 920.6 |
| | subunit D | | | | | |
| Senp3 | TSR1 20S rRNA accumulation | 2.1 | 913.9 | 1915.9 | 1474.6 | 477.2 |
| Tmtc2 | MYB binding protein (P160) 1a | 2.6 | 1140.0 | 2962.9 | 2200.7 | 459.8 |
| Fosb | T cell activation Rho GTPase activating | 2.4 | 1176.7 | 2794.4 | 489.3 | 704.2 |
| | protein | | | | | |
| Pdcd11 | RAB8B, member RAS oncogene family | 2.1 | 1189.5 | 2492.2 | 1671.3 | 2512.5 |
| Usp31 | DEAD (Asp-Glu-Ala-Asp) box | 2.4 | 1210.2 | 2928.0 | 2221.1 | 1098.2 |
| | polypeptide 21 | | | | | |
| Cdk8 | chaperonin containing Tcp1, subunit 4 | 2.3 | 1321.4 | 2989.7 | 2462.5 | 1294.8 |
| | (delta) | | | | | |
| Eftud2 | coiled-coil-helix-coiled-coil-helix domain | 2.3 | 1374.2 | 3171.2 | 2636.9 | 1008.9 |
| | containing 2 | | | | | |
| Fam60a | WD repeat domain 43 | 2.3 | 1727.6 | 3912.6 | 2927.5 | 1014.9 |

TABLE 7
Selection of preferred endogenous genes that are
constantly active during immune cell activation (dependent
or independent from T-cell activation).

| Symbol | Gene description |
|---|---|
| CD3G | CD3 gamma |
| Rn28s1 | 28S ribosomal RNA |
| Rn18s | 18S ribosomal RNA |
| Rn7sk | RNA, 7SK, nuclear |
| Actg1 | actin, gamma, cytoplasmic 1 |
| B2m | beta-2 microglobulin |
| Rpl18a | ribosomal protein L18A |
| Pabpc1 | poly(A) binding protein, cytoplasmic 1 |
| Gapdh | glyceraldehyde-3-phosphate dehydrogenase |
| Rpl19 | ribosomal protein L19 |
| Rpl17 | ribosomal protein L17 |
| Rplp0 | ribosomal protein, large, P0 |
| Cfl1 | cofilin 1, non-muscle |
| Pfn1 | profilin 1 |

TABLE 8
Selection of genes that are transiently
upregulated upon T-cell activation.

| Symbol | Gene description |
|---|---|
| Il3 | interleukin 3 |
| Il2 | interleukin 2 |
| Ccl4 | chemokine (C-C motif) ligand 4 |
| Il21 | interleukin 21 |
| Gp49a | glycoprotein 49 A |
| Nr4a3 | nuclear receptor subfamily 4, group A, member 3 |
| Lilrb4 | leukocyte immunoglobulin-like receptor, subfamily B, |
| | member 4 |
| Cd200 | CD200 antigen |
| Cdkn1a | cyclin-dependent kinase inhibitor 1A (P21) |
| Gzmc | granzyme C |
| Nr4a2 | nuclear receptor subfamily 4, group A, member 2 |
| Cish | cytokine inducible SH2-containing protein |
| Ccr8 | chemokine (C-C motif) receptor 8 |
| Lad1 | ladinin |
| Crabp2 | cellular retinoic acid binding protein II |

TABLE 9
Selection of genes that are upregulated over
more than 24 hours upon T-cell activation.

| Symbol | Description |
|---|---|
| Gzmb | granzyme B |
| Tbx21 | T-box21 |
| Pdcd1 | programmed cell death 1 |
| Plek | pleckstrin |
| Chek1 | checkpoint kinase 1 |
| Slamf7 | SLAM family member 7 |
| Zbtb32 | zinc finger and BTB domain containing 32 |
| Tigit | T cell immunoreceptor with Ig and ITIM domains |
| Lag3 | lymphocyte-activation gene 3 |
| Gzma | granzyme A |
| Wee1 | WEE 1 homolog 1 (S. pombe) |
| Il12rb2 | interleukin 12 receptor, beta 2 |
| Ccr5 | chemokine (C-C motif) receptor 5 |
| Eea1 | early endosome antigen 1 |
| Dtl | denticleless homolog (Drosophila) |

1-47. (canceled)

48. A method for engineering proteasome inhibitor resistant cells, wherein said method comprises the steps of:
providing primary immune cells;
performing gene editing of an endogenous gene in said primary immune cells with a sequence-specific endonuclease reagent selected from an RNA-guided endonuclease, a TALE-nuclease, and a Zinc-Finger-nuclease;
selecting cells that have acquired resistance to at least a LD50 dose of a proteasome inhibitor selected from bortezomib, carfilzomib, ixazomib, marizomib, delanzomib or oporozomib, and
expanding the selected cells.

49. The method of claim 48, wherein said cells are primary T-cells.

50. The method of claim 48, wherein the proteasome inhibitor is Bortezomib.

51. The method of claim 50, wherein the cells have acquired resistance to at least a dose of Bortezomib of ≥0.1 mg/m².

52. The method of claim 48, wherein the proteasome inhibitor is Carfilzomib.

53. The method of claim 52, wherein the cells have acquired resistance to at least a dose of Carfilzomib of ≥2 mg/m².

54. The method of claim 48, wherein the proteasome inhibitor is ixazomib.

55. The method of claim 54, wherein the cells have acquired resistance to at least a dose of ixazomib of ≥1 mg/m².

56. The method of claim 48, wherein the proteasome inhibitor is Marizomib.

57. The method of claim 48, wherein said primary immune cell is selected from the group consisting of CD4+T lymphocytes, CD8+T lymphocytes, NKT lymphocytes, a Tumor infiltrating Lymphocytes, TCR expressing cells and Treg lymphocytes, or a population thereof, or a progenitor thereof.

58. The method of claim 48, wherein said primary immune cell is a hematopoietic stem cell.

59. The method of claim 48, wherein the selected cells comprise an edited endogenous TCRalpha gene and/or TCRbeta gene.

60. The method of claim 48, wherein the selected cells comprise an exogenous polynucleotide sequence coding for a chimeric antigen receptor (CAR) and/or a modified TCR, specific for a molecule expressed at the surface of a pathological cell.

61. The method of claim 48, wherein the CAR is specific for CD19.

62. The method of claim 48, wherein said endogenous sequence encodes a protein selected from a proteasome subunit, a P-glycoprotein encoded by ATP-binding cassette sub-family B (ABCB) gene, a wnt glycoprotein, Interleukin-6 (IL-6), insulin-like growth factor-1 (IGF-1), insulin-like growth factor-1 receptor (IGF-1R), a proteasomal beta5i subunit low molecular weight protein 7 (LMP7), a cluster of differentiation(CD) 52 (CD52), CD274, transcription factor 4 (TCF-4), nuclear factor (erythroid-derived 2)-like (NRF2), a transcription factor Yin Yang 1 (YY1), transcription elongation factor B1 (TCEB1), TCEB2, RING-box protein 1 (RBX1), anaphase promoting complex subunit 11 (ANAPC11), Von Hippel-Lindau tumor suppressor (VHL), a DNA damage-binding protein 1 (DDB1), a Src family kinase, preferably Lyn, a Phosphatidyl Inositol 3 kinase (PI3K), a Protein kinase B (AKT), a mechanistic target of rapamycin (mTOR), a heat shock protein (Hsp), a proteasome maturation protein (POMP), a proteasome subunit (PSMB) protein, and a transcriptional activator of PSMB gene.

63. The method of claim 62, wherein said proteasome subunit is a proteasome β1-subunit selected from PSMB1, PSMB4, PSMB5, PSMB6, PSMA2, PSMA3, PSMA6, PSMA7, and PSMA8.

64. The method of claim 63, wherein said PSMB5 protein is mutated.

65. The method of claim 64, wherein said PSMB5 protein comprises at least one mutation among Thr21Ala, Ala49Thr, Ala50Val, Cys52Phe, Met451Ile, Cys63Phe and Arg24Cys.

66. The method of claim 48, comprising transfecting the cells with a library of sequence-specific reagents spanning a variety of endogenous genes sequences to inactivate those genes or integrate exogenous gene sequences prior to selecting the cells that have acquired resistance to the proteasome inhibitor.

67. The method of claim 48, wherein an exogenous sequence expressing a proteasome subunit or a mutated form thereof is introduced into the cells.

68. The method of claim 67, wherein said mutated form of proteasome subunit is PSMB5 that comprises at least one mutation selected among Thr21Ala, Ala49Thr, Ala50Val, Cys52Phe, Met451Ile, Cys63Phe and Arg24Cys.

69. The method of claim 67, wherein said exogenous sequence is integrated into an endogenous gene sequence with the effect of inactivating the expression of said endogenous gene.

70. The method of claim 48, wherein at least one endogenous gene of said immune cell encoding BIM, BAK, BIK, BAX, PRKAA1, CUL3, IPO4, Rab6B, STIP1, HECTD2, BAB14306.1, COPE, DMC1, NP002070, REXO1L1P, SURF6, PRKACA, PRKACG and EZH2 is inactivated.


SEQUENCE LISTING (4 sequences)

SEQ ID NO: 1 - DNA, 1254 bp, artificial sequence
  POMP-T2A-GFP
ggcgcgccagtcctccgacagactgagtcgcccgggggccaccatgaacgcaagggggctggggtccgaactgaaagatagtattcccgtcaccgaactgtccgcatcagggccatttgagagccacgatctgctgagaaagggctttagctgcgtgaagaacgagctgctgccatcccaccccctggagctgtctgagaagaacttccagctgaatcaggacaagatgaacttttccaccctgaggaatatccagggcctgttcgcccccctgaagctccagatggagtttaaggcagtgcagcaggtgcagcggctgcccttcctgagcagcagcaacctgtctctggacgtgctgaggggcaatgacgagacaatcggcttcgaggacatcctgaacgatcccagccagtccgaagtgatgggcgagcctcacctgatggtggagtacaagctgggcctgctgggcagcggcgagggcagaggctccctgctgacatgcggcgatgtggaggagaatcccggccctatgtccggcggagaggagctgttcgcaggaatcgtgcccgtgctgatcgagctggacggcgatgtgcacggccacaagttttctgtgcgcggagagggagagggcgacgccgattatggcaagctggagatcaagttcatctgtaccacaggcaagctgccagtgccctggcctaccctggtgaccacactgtgctacggcatccagtgttttgcccggtatccagagcacatgaagatgaacgacttctttaagagcgccatgcccgagggctacatccaggagaggacaatccagttccaggacgatggcaagtataagacccgcggcgaggtgaagtttgagggcgatacactggtgaaccggatcgagctgaagggcaaggacttcaaggaggatggcaatatcctgggccacaagctggagtactcttttaacagccacaacgtgtacatccgccccgacaaggccaacaatggcctggaggccaacttcaagaccaggcacaatatcgagggaggaggagtgcagctggcagaccactaccagacaaacgtgcctctgggcgatggccctgtgctgatcccaatcaatcactatctgtctacccagacaaagatcagcaaggaccggaatgaggccagagatcacatggtgctgctggaatctttctccgcttgttgtcacactcacgggatggacgaactgtatcgctaacctgcagg

SEQ ID NO: 2 - DNA, 1620 bp, artificial sequence
  PSMB5-T2A-GFP
ggcgcgccagtcctccgacagactgagtcgcccgggggccaccatggcactggcatccgtgctggagaggcccctgcctgtgaaccagaggggcttctttggcctgggcggcagagccgacctgctggatctgggcccaggctctctgagcgacggcctgtctctggcagcccctggctggggagtgcctgaggagccaggcatcgagatgctgcacggcaccacaaccctggccttcaagtttcggcacggcgtgatcgtggccgccgactctagagccacagccggcgcctatatcgccagccagaccgtgaagaaagtgatcgagatcaacccttacctgctgggaacaatggccggaggagccgccgattgcagcttttgggagaggctgctggccaggcagtgtcgcatctatgagctgcggaacaaggagagaatcagcgtggctgccgcctccaagctgctggccaatatggtgtaccagtataagggcatgggcctgagcatgggcacaatgatctgcggatgggacaagaggggccccggcctgtactatgtggattctgagggcaatcgcatctctggcgccaccttcagcgtgggcagcggcagcgtgtacgcctacggcgtgatggacagaggctacagctatgatctggaggtggagcaggcttacgacctggcccggcgggccatctaccaggccacctatagggatgcctactccggcggagcagtgaacctgtatcacgtgcgggaggacggctggatcagagtgagcagcgacaatgtggccgatctgcacgagaagtacagcggctccacaccaggctccggcgagggccgcggctctctgctgacctgcggcgatgtggaggagaacccaggccccatgtctggcggagaggagctgttcgcaggaatcgtgcccgtgctgatcgagctggacggcgatgtgcacggccacaagtttagcgtgcgcggagagggagagggcgacgccgattacggcaagctggagatcaagttcatctgtacaaccggcaagctgcctgtgccctggcccacactggtgacaaccctgtgctatggcatccagtgttttgcccggtaccctgagcacatgaagatgaatgacttctttaagtccgccatgccagagggctatatccaggagcggaccatccagttccaggacgatggcaagtacaagacaagaggcgaggtgaagtttgagggcgataccctggtgaacaggatcgagctgaagggcaaggacttcaaggaggatggcaatatcctgggccacaagctggagtattcctttaactctcacaacgtgtacatccgccccgacaaggccaacaatggcctggaggccaactttaagacacggcacaatatcgagggaggaggagtgcagctggcagaccactatcagaccaacgtgcctctgggcgatggccccgtgctgatccctatcaatcactacctgagcacacagaccaagatcagcaaggacaggaatgaggcccgcgatcacatggtgctgctggagtctttcagcgcctgctgtcacacccacggcatggatgagctgtacagatgacctgcagg

SEQ ID NO: 3 - DNA, 1620 bp, artificial sequence
  mutated PSMB5-T2A-GFP
ggcgcgccagtcctccgacagactgagtcgcccgggggccaccatggcactggcatccgtgctggagaggcccctgcctgtgaaccagaggggcttctttggcctgggcggcagagccgacctgctggatctgggcccaggctctctgagcgacggcctgtctctggcagcccctggctggggagtgcctgaggagccaggcatcgagatgctgcacggcaccacaaccctggccttcaagtttcggcacggcgtgatcgtggccgccgactctagagccacagccggcgcctatatcgccagccagaccgtgaagaaagtgatcgagatcaacccttacctgctgggaacaatggccggaggaacagtggattgcagcttttgggagaggctgctggccaggcagtgtcgcatctatgagctgcggaacaaggagagaatcagcgtggctgccgcctccaagctgctggccaatatggtgtaccagtataagggcatgggcctgagcatgggcacaatgatctgcggatgggacaagaggggccccggcctgtactatgtggattctgagggcaatcgcatctctggcgccaccttcagcgtgggcagcggcagcgtgtacgcctacggcgtgatggacagaggctacagctatgatctggaggtggagcaggcttacgacctggcccggcgggccatctaccaggccacctatagggatgcctactccggcggagcagtgaacctgtatcacgtgcgggaggacggctggatcagagtgagcagcgacaatgtggccgatctgcacgagaagtacagcggctccacaccaggctccggcgagggccgcggctctctgctgacctgcggcgatgtggaggagaacccaggccccatgtctggcggagaggagctgttcgcaggaatcgtgcccgtgctgatcgagctggacggcgatgtgcacggccacaagtttagcgtgcgcggagagggagagggcgacgccgattacggcaagctggagatcaagttcatctgtacaaccggcaagctgcctgtgccctggcccacactggtgacaaccctgtgctatggcatccagtgttttgcccggtaccctgagcacatgaagatgaatgacttctttaagtccgccatgccagagggctatatccaggagcggaccatccagttccaggacgatggcaagtacaagacaagaggcgaggtgaagtttgagggcgataccctggtgaacaggatcgagctgaagggcaaggacttcaaggaggatggcaatatcctgggccacaagctggagtattcctttaactctcacaacgtgtacatccgccccgacaaggccaacaatggcctggaggccaactttaagacacggcacaatatcgagggaggaggagtgcagctggcagaccactatcagaccaacgtgcctctgggcgatggccccgtgctgatccctatcaatcactacctgagcacacagaccaagatcagcaaggacaggaatgaggcccgcgatcacatggtgctgctggagtctttcagcgcctgctgtcacacccacggcatggatgagctgtacagatgacctgcagg

SEQ ID NO: 4 - protein, 263 aa, Homo sapiens
MALASVLERPLPVNQRGFFGLGGRADLLDLGPGSLSDGLSLAAPGWGVPEEPGIEMLHGTTTLAFKFRHGVIVAADSRATAGAYIASQTVKKVIEINPYLLGTMAGGAADCSFWERLLARQCRIYELRNKERISVAAASKLLANMVYQYKGMGLSMGTMICGWDKRGPGLYYVDSEGNRISGATFSVGSGSVYAYGVMDRGYSYDLEVEQAYDLARRAIYQATYRDAYSGGAVNLYHVREDGWIRVSSDNVADLHEKYSGSTP